(19) United States
(12) Patent Application Publication
(10) Pub. No.: US 2024/0200003 A1
(43) Pub. Date: Jun. 20, 2024

(54) DEVICES AND METHODS FOR IN VITRO MODELING OF METASTATIC CANCER

(71) Applicant: XYLYX BIO, INC., Brooklyn, NY (US)

(72) Inventors: John O'NEILL, Brooklyn, NY (US); Igal GERMANGUZ, Brooklyn, NY (US); Evelyn ARANDA, Brooklyn, NY (US); Jennifer XIONG, Brooklyn, NY (US); Natalia KISSEL, Brooklyn, NY (US); Alexandra NICHOLS, Brooklyn, NY (US); Drew DALY, Brooklyn, NY (US)

(21) Appl. No.: 17/997,829

(22) PCT Filed: May 5, 2021
(86) PCT No.: PCT/US2021/030840

§ 371 Date: Oct. 4, 2023

Related U.S. Application Data
(60) Provisional application No. 63/020,160, filed on May 5, 2020.

(51) Int. Cl. C12M 3/00; C12M 3/06; C12N 5/09; G01N 33/50

(52) U.S. Cl. CPC C12M 21/08; C12M 23/16; C12N 5/0693; G01N 33/5082; G01N 33/5091; C12N 2501/999; C12N 2513/00; C12N 2533/90

(57) ABSTRACT

A cell culture platform for modeling metastatic cancer is disclosed. The platform comprises one or more cell culture vessels comprising a plurality of compartments. Each compartment houses a substrate include a decellularized tissue-specific extracellular matrix derived from tissue of a different anatomical region. Each tissue-specific extracellular matrix comprises a homogenous mixture of macromolecule fragments including collagen, elastin, and glycosaminoglycan. A kit for culturing cells in biomimetic environments is also disclosed. The kit comprises a plurality of substrate precursors and at least one reagent. Each substrate precursor comprises a decellularized tissue-specific extracellular matrix derived from tissue of a different anatomical region. The tissue-specific extracellular matrix comprises a homogenous mixture of macromolecule fragments including collagen, elastin, and glycosaminoglycan. The reagent is configured to configured to convert each substrate precursor into a substrate adapted for culturing cells thereon. Methods of assessing a tumor-associated response of a cancer colony are also disclosed herein.

### CROSS-REFERENCE TO RELATED APPLICATIONS

This application is a National Phase application of PCT Application No. PCT/US2021/030840 entitled “Devices and Methods for in vitro Modeling of Metastatic Cancer,” filed May 5, 2021, which claims the benefit of priority to U.S. Provisional Application No. 63/020,160 entitled “Devices and Methods for in vitro Modeling of Metastatic Cancer,” filed May 5, 2020, each of which are incorporated herein by reference in their entirety.

### TECHNICAL FIELD

The present disclosure relates generally to devices, methods, and kits related to in vitro cell models that include biomaterials derived from tissue-specific extracellular matrix. More particularly, the present disclosure relates to devices, methods, and kits for in vitro modeling of metastatic cancer. The disclosed techniques may be applied to, for example, breast cancer, as well as other types of cancer.

### BACKGROUND

Cancer is a well-known, serious disease characterized by uncontrolled growth of abnormal cells in a tissue. Although advancements have been made in the diagnosis and treatment of various types of cancers, the nature of cancer necessitates specific study and solution with respect to each type, location, and stage of cancer. As such, there is an ongoing need for improved diagnosis and treatment of various types of cancer.

Metastasis is a pathological process in which tumor cells (i.e. primary tumor cells) depart the primary tumor site and colonize a secondary site in a different tissue or organ. For example, breast cancer cells often depart the breast tissue and metastasize to other tissue sites such as the bone, liver, and lung. Metastasis causes approximately 90% of cancer deaths, with metastatic breast cancer being a leading cause of death from cancer. As cell-cell and cell-matrix interactions are important physiological determinants of cell growth, survival, and transformation, the extracellular matrix plays a critical role in metastatic invasion and colonization. The localization of metastatic cancer at a secondary site is not a random process, but rather heavily dependent on the physical interactions and molecular communication between tumor cells, resident cells, and the surrounding environment.

In its native environment, extracellular matrix (ECM) is a scaffold with tissue-specific cues (e.g., molecular, structural, biomechanical) that provides structure for cell maintenance and growth and mediates cell proliferation, differentiation, gene expression, migration, orientation, and assembly. In its native environment, ECM comprises an interlocking mesh of components including but not limited to viscous proteoglycans (e.g., heparin sulfate, keratin sulfate, and chondroitin sulfate) that provide cushioning, collagen and elastin fibers that provide strength and resilience, and soluble multiadhesive proteins (e.g., fibronectin and laminin) that bind the proteoglycans and collagen fibers to cell receptors. Native extracellular matrix also commonly includes hyaluronic acid and cellular adhesion molecules (CAMs) such as integrins, cadherins, selectins, and immunoglobulins. Each tissue may perform a unique set of roles and accordingly requires different properties in its extracellular matrix. As such, the precise composition of ECM varies from tissue to tissue, forming specialized ECM niches (i.e., tissue-specific extracellular matrix or TS-ECM).

The complexity of the ECM has proven difficult to recapitulate in its entirety outside of its native environment. Mimicking just the ECM structure using synthetic biomaterials or mimicking composition by adding purified ECM components is possible. While offering structural mimics, synthetic biomaterials can alter cell behavior (i.e., proliferation, differentiation, gene expression, migration, orientation, and assembly) in vitro and potentially generate cytotoxic by-products at the site of implantation, leading to poor wound healing or an inflammatory environment. An alternative to synthetic biomaterials is to directly isolate the native ECM from the tissue of interest via the removal of cells and cellular remnants. ECM-derived biomaterials can be processed into scaffolds (such as acellular scaffolds or sponges) with appropriate compositions and structures for cell culture and tissue engineering. ECM scaffolds can be derived from various sources such as human and animal; fetal, juvenile, and adult; healthy, diseased, and transgenic tissues.

Due to the role of ECM in the development of metastatic cancer, TS-ECM is a key component for accurately modeling metastatic cancer and evaluating potential treatments. However, in-vitro metastasis models incorporating TS-ECM components are not currently available. Conventional models utilized at the early stages of the pre-clinical evaluation process utilize tissue culture plastic, Matrigel, and other non-equivalent substrates as a substitute for TS-ECM, leading to misleading and non-translatable results during important development phases. As such, there is a significant need for in vitro models that provide physiologically relevant results by recapitulating the metastatic niche environment.

### SUMMARY

Embodiments of the invention are directed to a cell culture platform for modeling metastatic cancer comprising: one or more cell culture vessels comprising a plurality of compartments, each compartment housing a substrate adapted for culturing cells thereon, wherein each substrate comprises a decellularized tissue-specific extracellular matrix derived from tissue of a different anatomical region, wherein each tissue-specific extracellular matrix comprises a homogenous mixture of macromolecule fragments including collagen, elastin, and glycosaminoglycan.

Embodiments of the invention are directed to a kit for culturing cells in biomimetic environments, the kit comprising: a plurality of substrate precursors, each substrate precursor comprising a decellularized tissue-specific extracellular matrix, wherein each tissue-specific extracellular matrix is derived from tissue of a different anatomical region and comprises a homogenous mixture of macromolecule fragments including collagen, elastin, and glycosaminoglycan; and at least one reagent configured to convert each substrate precursor into a substrate adapted for culturing cells thereon.

Embodiments of the invention are directed to a method of assessing a cancer-associated response of one or more cancer colonies, the method comprising: providing one or more cell culture vessels comprising a plurality of substrates arranged in a compartmentalized manner, each substrate comprising a decellularized tissue-specific extracellular matrix derived from tissue of a different anatomical region; culturing cancer cells to form a cancer colony on each substrate, wherein the cancer cells are foreign to the tissue-specific extracellular matrix of at least one of the plurality of substrates, thereby forming at least one metastatic cancer colony; and assessing at least one cancer-associated response of each cancer colony.

Embodiments of the invention are directed to a method of assessing a response of one or more cancer colonies to a drug, the method comprising: providing one or more cell culture vessels comprising a plurality of substrates arranged in a compartmentalized manner, each substrate comprising a decellularized tissue-specific extracellular matrix derived from tissue of a different anatomical region; culturing cancer cells to form a cancer colony on each substrate, wherein the cancer cells are foreign to the tissue-specific extracellular matrix of at least one of the plurality of substrates, thereby forming at least one metastatic cancer colony; contacting each cancer colony with a drug; and assessing the response by each cancer colony to the drug.

Embodiments of the invention are directed to a method of assessing cell migration of a primary cancer colony, the method comprising: providing one or more cell culture vessels comprising: a first substrate in a first compartment of the one or more cell culture vessels, one or more second substrates in one or more second compartments of the one or more cell culture vessels, wherein the first substrate and the one or more second substrates each comprise a decellularized tissue-specific extracellular matrix derived from tissue of a different anatomical region, wherein the first substrate and the one or more second substrates are arranged in a compartmentalized manner and in fluid communication through one or more fluidic channels; culturing cancer cells on the first substrate, wherein the cancer cells are native to the tissue-specific extracellular matrix of the first substrate, thereby forming the primary cancer colony, wherein the one or more fluidic channels are configured to permit cell migration from the first substrate to the one or more second substrates, wherein the cancer cells are adapted to form one or more metastatic cancer colonies on the one or more second substrates; and assessing the cell migration of the primary cancer colony from the first substrate to the one or more second substrates.

### BRIEF DESCRIPTION OF THE DRAWINGS

The accompanying drawings, which are incorporated in and form a part of the specification, illustrate the embodiments of the invention and together with the written description, serve to explain the principles, characteristics, and features of the invention. In the drawings:

FIG. 1 depicts an illustrative diagram of a method of making a cell culture platform in accordance with an embodiment.

FIGS. 2A-2D depict an exemplary approach to produce a cell culture platform in accordance with an embodiment.

FIGS. 3A-3J depict an exemplary evaluation of reconstituted tissue-specific extracellular matrix substrate in accordance with an embodiment.

FIGS. 4A-4C depict an exemplary evaluation of breast cancer cells in tissue-specific extracellular matrix substrates in accordance with an embodiment.

FIGS. 5A-5B depict an exemplary evaluation of drug response of breast cancer cells in tissue-specific extracellular matrix substrates in accordance with an embodiment.

FIG. 6 depicts lung cancer cell models in accordance with an embodiment.

FIGS. 7A-7B depict an exemplary evaluation of responses of lung cancer cells to drugs (KPT-185 and Etoposide) in lung-specific extracellular matrix substrates in accordance with an embodiment.

FIG. 8 depicts an exemplary evaluation of response of lung cancer cells to a drug (Erlotinib) in lung-specific extracellular matrix substrates in accordance with an embodiment.

FIG. 9 depicts an exemplary evaluation of migration of lung cancer cells in tissue-specific extracellular matrix substrates in accordance with an embodiment.

FIG. 10 depicts an exemplary evaluation of invasion of lung cancer cells in lung-specific extracellular matrix substrates in accordance with an embodiment.

FIG. 11 depicts an exemplary evaluation of gene expression of metastatic breast cancer cells on tissue-specific extracellular matrix substrates in accordance with an embodiment.

FIG. 12 depicts an exemplary evaluation of extracellular matrix remodeling in tissue-specific extracellular matrix substrates in accordance with an embodiment.

FIG. 13 depicts an exemplary evaluation of drug response of lung cancer cells in lung-specific extracellular matrix substrates in accordance with an embodiment.

FIG. 14 depicts an exemplary evaluation of migration of jacket and A549 cells in lung-specific extracellular matrix substrates in accordance with an embodiment.

FIG. 15 depicts an exemplary evaluation of cancer-related gene expression of cancer cells in lung-specific extracellular matrix substrates in accordance with an embodiment.

FIG. 16 depicts an exemplary evaluation of drug response of breast cancer cells in extracellular matrix substrates in accordance with an embodiment.

FIGS. 17A-17H depict an exemplary evaluation of a human cirrhotic liver ECM for an in matrico hepatocellular carcinoma model in accordance with an embodiment.

### DETAILED DESCRIPTION

Various aspects now will be described more fully hereinafter. Before the present devices, systems, compositions, and methods are described, it is to be understood that this invention is not limited to the particular devices, systems, compositions, processes, or methodologies described, as these may vary. It is also to be understood that the terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described.

It must be noted that as used herein and in the appended claims, the singular forms “a”, “an”, and “the” include plural reference unless the context clearly dictates otherwise. Thus, for example, reference to a “fibroblast” is a reference to one or more fibroblasts and equivalents thereof known to those skilled in the art, and so forth.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

It must also be noted that the word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, where features of the disclosure are described in terms of Markush groups, those skilled in the art will recognize that the disclosure is also thereby described in terms of any individual member or subgroup of members of the Markush group.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera).

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

All percentages, parts, and ratios are based upon the total weight of the materials and all measurements made are at about 25° C., unless otherwise specified.

For convenience, certain terms employed in the specification, examples, and claims are collected here. Unless defined otherwise, all technical and scientific terms used in this disclosure have the same meanings as commonly understood by one of ordinary skill in the art to which this disclosure belongs.

Unless defined otherwise, all technical and scientific terms used herein have the same meanings as commonly understood by one of ordinary skill in the art, including scientists, engineers, researchers, industrial designers, laboratory and production technicians and assistants and users of the systems and methods for their designed purposes.

The term “animal” as used herein includes, but is not limited to, humans and non-human vertebrates such as wild, domestic, and farm animals.

The term “inhibiting” includes the administration of a compound of the present invention to prevent the onset of the symptoms, alleviating the symptoms, reducing the symptoms, delaying or decreasing the progression of the disease and/or its symptoms, or eliminating the disease, condition or disorder.

As used herein, the term “therapeutic” means an agent utilized to treat, combat, ameliorate, prevent, or improve an unwanted condition or disease of a patient. In part, embodiments of the present invention are directed to the treatment of cancer or the decrease in proliferation of cells.

Generally speaking, the term “tissue” refers to any aggregation of similarly specialized cells which are united in the performance of a particular function. As used herein, “tissue”, unless otherwise indicated, refers to tissue which includes elastin as part of its necessary structure and/or function. For example, connective tissue which is made up of, among other things, collagen fibrils and elastin fibrils satisfies the definition of “tissue” as used herein. Additionally, elastin appears to be involved in the proper function of blood vessels, veins, and arteries in their inherent visco-elasticity.

The terms “metastasis,” “metastasize,” and “metastatic” as used herein refer to the spread and growth of cancer cells in a foreign tissue (i.e., a tissue to which the cells are not native). For example, where breast cancer cells spread to the lungs and form a tumor therein, the cancer cells are metastatic breast cancer cells and not lung cancer cells. In vivo, metastasis typically involves cancer cells breaking away from the site where they first formed (i.e., the primary tumor) and traveling to a new organ or tissue via the blood or lymph system. The primary cancer cells take residence in the new organ or tissue to form a metastatic colony. For in vivo applications such as cell culturing and modeling, metastasis may also include a colony of cancer cells that reside in an environment that emulate a foreign tissue. For example, breast cancer cells cultured in vivo in an environment that emulates lung tissue may be referred to as metastatic breast cancer cells and may form a metastatic colony of breast cancer.

The term “disorder” is used in this disclosure to mean, and is used interchangeably with, the terms disease, condition, or illness, unless otherwise indicated.

The term “patient” and “subject” are interchangeable and may be taken to mean any living organism which may be treated with compounds of the present invention. As such, the terms “patient” and “subject” may include, but is not limited to, any non-human mammal, primate or human. A subject can be a mammal such as a primate, for example, a human. The term “subject” includes domesticated animals such as cats, dogs, etc., livestock (e.g., cattle, horses, swine, sheep, goats, etc.), and laboratory animals (e.g., mice, rabbits, rats, gerbils, guinea pigs, possums, etc.). In some embodiments, the patient or subject is an adult, child or infant. In some embodiments, the patient or subject is a human.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure. Nothing herein is to be construed as an admission that the invention is not entitled to antedate such disclosure by virtue of prior invention.

### Cell Culture Platform

Embodiments of the invention are directed to an in vitro cell culture platform for modeling metastatic cancer. The cell culture platform may comprise a cell culture vessel (e.g., a culture plate) having one or more cell culture substrates, each comprising a different tissue-specific extracellular matrix that recapitulates the composition, mechanics, and cell-matrix interactions specific to a particular tissue. The cell culture platform may be utilized with cancer cells cultured in the TS-ECM to emulate a metastatic niche environment. As discussed herein, in some embodiments the cell culture vessel includes two or more cell culture substrates to emulate multiple different niche environments.

The cell culture substrates (also referred to herein as “TS-ECM substrates” or “substrates”) may be derived from a variety of tissue types, and thus the resulting TS-ECM substrates may emulate the niche environment of various tissues. In some embodiments, the TS-ECM may emulate common sites of metastasis. For example, the TS-ECM may be selected from bone-specific ECM, lung-specific ECM, and liver-specific ECM. In additional embodiments, the TS-ECM may be selected from additional niche environments, such as brain-specific ECM, kidney-specific extracellular matrix, skin-specific extracellular matrix, intestine-specific extracellular matrix, heart-specific extracellular matrix, and lymph-specific extracellular matrix. In still additional embodiments, the TS-ECM may emulate a niche environment specific to another tissue. For example, the tissue may be selected from the adrenal gland, amnion, bladder, blood vessel, breast, cartilage, chorion, connective tissue, esophagus, eye, fat, larynx, ligament, microvasculature, muscle, mouth, omentum, ovary, fallopian tube, thyroid, parathyroid, large intestine, small intestine, pancreas, peritoneum, pharynx, placenta membrane, prostate, rectum, smooth muscle, spinal cord, spinal fluid, spleen, stomach, tendon, testes, thymus, umbilical cord, uterus, vagina, or Wharton's Jelly. In some embodiments, the TS-ECM may emulate a region of the anatomy, an organ, or a region of an organ. For example, left and right lungs have unique anatomies and may represent unique TS-ECMs which may be utilized individually or together for direct comparison. In another example, a TS-ECM may represent the large intestine or it may more specifically represent the colon or the rectum.

The tissues may be derived from a variety of non-metastatic tissue sources, i.e., tissues prior to the development of metastasis. In some embodiments, the tissue source is selected from a human source and an animal source. For example, the tissue may be porcine (i.e., sourced from a pig) or any other animal tissue known to have clinical relevance. In some embodiments, the tissue source is selected from fetal tissue, juvenile tissue, and adult tissue. In some embodiments, the tissue source is selected from healthy tissue, diseased tissue, transgenic tissue, or tissue having a specific disorder or health condition. For example, in some embodiments, the tissue source is fibrotic tissue (i.e., exhibiting tissue fibrosis). The resulting TS-ECM is representative of extracellular matrix from the tissue source, or more generally from tissue having the same relevant characteristics as the tissue source (e.g., juvenile human lung tissue will yield lung-specific ECM representative of a juvenile human's lung tissue).

In some embodiments, the cell culture vessel comprises a tissue culture plate. In some embodiments, the cell culture vessel may be a petri dish or other dish. In some embodiments, the cell culture vessel comprises a flask. Additional types of cell culture vessel as would be known to one having an ordinary level of skill in the art are also contemplated herein. The cell culture vessel may comprise one or more divided regions to be utilized for individual TS-ECM substrates. For example, a tissue culture plate may comprise one or more wells. In some embodiments, the plate comprises 1 well, 3 wells, 6 wells, 12 wells, 24 wells, 48 wells, 96 wells, 384 wells, greater than 384 wells, or any individual value or any range between any two values therein.

In some embodiments, the in vitro cell culture platform has a shelf life of about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, or any individual value or any range between any two values therein.

In some embodiments, the in vitro cell culture platform comprises a plurality of TS-ECM substrates. The cell culture platform may be a culture plate having a plurality of divided regions (e.g., wells), where each region includes a TS-ECM substrate. In some embodiments, the plurality of TS-ECM substrates may include a variety of different tissue-specific extracellular matrices in order to emulate multiple niche environments in a single platform. For example, a culture plate may include one or more first wells comprising a first TS-ECM substrate, one or more second wells comprising a second TS-ECM substrate, and one or more third wells comprising a third TS-ECM substrate, wherein each TS-ECM substrate is a different TS-ECM. In some embodiments, the first TS-ECM substrate comprises bone-specific ECM, the second TS-ECM substrate comprises lung-specific ECM, and the third TS-ECM substrate comprises liver-specific ECM. However, any combination of TS-ECM substrates disclosed herein is contemplated. While a combination of three different TS-ECM substrates is demonstrated, it should be understood that other quantities are contemplated. A culture plate may comprise two, three, four, five, or more different TS-ECM substrates.

In some embodiments, combinations of TS-ECM substrates are selected based on common sites of metastasis for a particular tumor type. In some embodiments, a cell culture platform for modeling breast cancer cells comprises one or more different TS-ECM substrates, each emulating a niche environment selected from bone tissue, liver tissue, lung tissue, and brain tissue. In some embodiments, a cell culture platform for modeling lung cancer cells comprises one or more different TS-ECM substrates each emulating a niche environment selected from bone tissue, liver tissue, opposite lung tissue (e.g., where the cancer cells are from a left lung, the TS-ECM emulates right lung tissue), brain tissue, and adrenal gland tissue. In some embodiments, a cell culture platform for modeling liver cancer cells comprises one or more different TS-ECM substrates, each emulating a niche environment selected from lung tissue, kidney tissue, and lymph tissue (e.g., portal lymph nodes). In some embodiments, a cell culture platform for modeling bone cancer cells comprises a TS-ECM substrate emulating a niche environment specific to lung tissue. In some embodiments, a cell culture platform for modeling brain cancer cells comprises one or more different TS-ECM substrate, each emulating a niche environment selected from spinal cord tissue and spinal fluid.

In additional embodiments, a cell culture platform for modeling bladder cancer cells comprises one or more different TS-ECM substrates, each emulating a niche environment selected from bone tissue, liver tissue, and lung tissue. In some embodiments, a cell culture platform for modeling colon cancer cells and/or rectal cancer cells comprises one or more different TS-ECM substrates, each emulating a niche environment selected from liver tissue, lung tissue, and peritoneal tissue. In some embodiments, a cell culture platform for modeling esophageal cancer cells comprises one or more different TS-ECM substrates, each emulating a niche environment selected from bone tissue, liver tissue, lung tissue, lymph node tissue, and stomach tissue. In some embodiments, a cell culture platform for modeling fallopian tube cancer cells comprises one or more different TS-ECM substrates, each emulating a niche environment selected from bone tissue, liver tissue, lung tissue, kidney tissue, brain tissue, peritoneal tissue, ovarian tissue, and uterine tissue. In some embodiments, a cell culture platform for modeling gallbladder cancer cells comprises one or more different TS-ECM substrates, each emulating a niche environment selected from liver tissue, pancreatic tissue, and lymph node tissue. In some embodiments, a cell culture platform for modeling kidney cancer cells comprises one or more different TS-ECM substrates, each emulating a niche environment selected from bone tissue, liver tissue, lung tissue, brain tissue, adrenal gland tissue, ovarian tissue, and testicular tissue. In some embodiments, a cell culture platform for modeling blood or bone marrow cancer cells (i.e., leukemia) comprises one or more different TS-ECM substrates, each emulating a niche environment selected from liver tissue, spleen tissue, spinal fluid, lymph node tissue, and testicular tissue. In some embodiments, a cell culture platform for modeling mouth cancer cells comprises one or more different TS-ECM substrates, each emulating a niche environment selected from lung tissue and lymph tissue (e.g., neck lymph nodes). In some embodiments, a cell culture platform for modeling oral and/or oropharyngeal cancer cells comprises one or more different TS-ECM substrates, each emulating a niche environment selected from lung tissue, kidney tissue, neck tissue, throat tissue, and prostate tissue. In some embodiments, a cell culture platform for modeling ovarian cancer cells comprises one or more different TS-ECM substrates, each emulating a niche environment selected from liver tissue, lung tissue, spleen tissue, peritoneal tissue, and fallopian tube tissue. In some embodiments, a cell culture platform for modeling pancreatic cancer cells comprises one or more different TS-ECM substrates, each emulating a niche environment selected from liver tissue, lung tissue, and peritoneal tissue. In some embodiments, a cell culture platform for modeling prostate cancer cells comprises one or more different TS-ECM substrates, each emulating a niche environment selected from bone tissue, liver tissue, lung tissue, and adrenal gland tissue. In some embodiments, a cell culture platform for modeling skin cancer cells comprises one or more different TS-ECM substrates, each emulating a niche environment selected from bone tissue, liver tissue, lung tissue, brain tissue, skin tissue, and muscular tissue. In some embodiments, a cell culture platform for modeling stomach cancer cells comprises one or more different TS-ECM substrates, each emulating a niche environment selected from liver tissue, lung tissue, and peritoneal tissue. In some embodiments, a cell culture platform for modeling testicular cancer cells comprises one or more different TS-ECM substrates, each emulating a niche environment selected from liver tissue and lymph node tissue. In some embodiments, a cell culture platform for modeling throat cancer cells comprises one or more different TS-ECM substrates, each emulating a niche environment selected from bone tissue and lung tissue. In some embodiments, a cell culture platform for modeling thyroid cancer cells comprises one or more different TS-ECM substrates, each emulating a niche environment selected from bone tissue, liver tissue, and lung tissue. In some embodiments, a cell culture platform for modeling urethral cancer cells comprises one or more different TS-ECM substrates, each emulating a niche environment selected from bone tissue, liver tissue, lung tissue, kidney tissue, and lymph node tissue. In some embodiments, a cell culture platform for modeling uterine cancer cells comprises one or more different TS-ECM substrates, each emulating a niche environment selected from bone tissue, liver tissue, lung tissue, peritoneal tissue, rectal tissue, bladder tissue, fallopian tube tissue, and vaginal tissue.

In still additional embodiments, a cell culture platform for modeling non-Hodgkin lymphoma cells comprises one or more different TS-ECM substrates, each emulating a niche environment selected from bone tissue, liver tissue, and lung tissue. In some embodiments, a cell culture platform for modeling multiple myeloma cells comprises one or more different TS-ECM substrates, each emulating a niche environment selected from central nervous system tissue (e.g., brain, spinal cord, spinal fluid) and blood. In some embodiments, a cell culture platform for modeling neuroblastoma cells comprises one or more different TS-ECM substrates, each emulating a niche environment selected from liver tissue and adrenal gland tissue. In some embodiments, a cell culture platform for modeling ocular melanoma cells comprises one or more different TS-ECM substrates, each emulating a niche environment selected from bone tissue, liver tissue, and lung tissue. In some embodiments, a cell culture platform for modeling sarcoma cells comprises a TS-ECM substrate emulating a niche environment specific to lung tissue. Additional types of cancer cells and/or additional sets of TS-ECM substrates are contemplated herein as would be known to one having an ordinary level of skill in the art.

In some embodiments, each TS-ECM substrate of the cell culture platform is segregated, i.e., completely physically separated from other TS-ECM substrates. The physical separation must be capable of preventing cell transfer between the TS-ECM substrates, co-mingling of cell culture components, interaction, cross-contamination, or any other influence of one substrate or culture upon another. In some embodiments, the segregation comprises a barrier such as a wall between the TS-ECM substrates. For example, as described, a tissue culture plate with a plurality of wells may be utilized such that the walls of the wells serve as a physical barrier between the TS-ECMs. Other types of barriers may be utilized as would be known to one having an ordinary level of skill in the art. In some embodiments, an adequate amount of physical spacing between TS-ECM substrates may provide sufficient segregation. For example, as described above, a tissue culture plate may include divided regions which are adequately spaced to provide for individual TS-ECM substrates. Further, in some embodiments, multiple plates or vessels may be utilized, where one or more TS-ECMs are provided on each plate or vessel in order to provide segregation. Various additional manners of providing physical separation between substrates as would be known to one having an ordinary level of skill in the art are contemplated herein.

In additional embodiments, each TS-ECM substrate may be compartmentalized, i.e., physically separated from the other TS-ECM substrates to prevent intermixing in a manner that would substantially alter the composition of any of the TS-ECM substrates. Compartmentalized TS-ECM substrates may include a means of fluid communication therebetween. For example, the compartmentalization may allow for some cell transfer, interaction, or other influence of one substrate or culture upon another (e.g., transfer of some molecules or creation of a gradient therebetween). In some embodiments, the TS-ECM substrates may be housed in physically separated compartments as described above (e.g., connected vessels, connected chambers of a vessel, etc.) except with fluid channels extending between the compartments. In some embodiments, the compartments comprise microfluidic chambers on a vessel such as chip (e.g., an organ-on-a-chip system). In some embodiments, each compartment comprises a printed bio-ink in a region of a vessel such as a chip. Further, the fluid communication between compartments may be formed in a variety of manners. In some embodiments, the compartments communicate via interconnecting channels spanning between the compartments. For example, the channels may be microfluidic channels. In some embodiments, the compartments are separated by a porous membrane that allows fluid communication therebetween. The fluid communication may be configured to allow transport of fluids, molecules, cells, or a combination thereof. Additionally, the fluid communication may be arranged in a variety of manners. In some embodiments, each of the additional compartments directly fluidly communicate with the first compartment in parallel circuit arrangement. For example, the compartments may be arranged in a hub-and-spoke arrangement where the first compartment serves as a central hub having direct fluid communication with each of the radially arranged additional compartments (i.e., spokes). However, the same structural connectivity may be formed with different physical arrangements. In additional embodiments, the first compartment and the additional compartments directly communicate in a series circuit arrangement (i.e., arranged in a chain) such that some additional compartments indirectly communicate with the first compartment (i.e., fluid communication occurs through a directly communicating compartment). Combinations of parallel and series connections are also contemplated herein. In some embodiments, at least one of the additional compartments directly communicate with the first compartment while the remaining additional compartments indirectly communicate with the first compartment. Several layers of interconnectivity may be formed in this manner. In some embodiments, the interconnectivity may mimic a biological system. For example, the TS-ECMs and the interconnectivity therebetween may mimic the interconnectivity of parts of an organ, a plurality of organs, and/or an organ system.

The TS-ECM may be processed and provided in a variety of substrate formats. In some embodiments, the format of the TS-ECM substrate may be selected from a hydrogel, a scaffold (e.g., an acellular scaffold), a surface coating, a sponge, fibers (e.g., electrospun fibers), liquid solution, media supplement, and bio-ink (e.g., printable bio-ink).

The TS-ECM has a specified composition that emulates the ECM found in a specific native tissue. As such, the composition of each TS-ECM may vary. Each TS-ECM may comprise a different combination of proteoglycans, collagens, elastins, multiadhesive proteins, hyaluronic acid, CAMs, and additional components. Each of these components may have subtypes, the presence of each of which may vary from one TS-ECM to another TS-ECM. Each TS-ECM may be characterized by the presence or absence of one or more components. Further, the concentration of each component may vary from one TS-ECM to another TS-ECM. These variations result in each TS-ECM having unique physical characteristics, such as architecture and stiffness, and unique cell interaction characteristics, such as gene expression, ECM remodeling, and cell proliferation.

In some embodiments, bone-specific ECM may comprise about 580-620 μg/mL collagens, about 40-50 μg/mL elastins, and about 10-20 μg/mL glycosaminoglycans. In some embodiments, the bone-specific ECM has an elastic modulus of about 6.6 kPa. However, the elastic modulus may be about 6 to about 25 kPa, about 6 to about 10⁵ kPa, or individual values or ranges therebetween. In some embodiments, the elastic modulus may be similar to the elastic modulus of natural bone tissue. In some embodiments, the collagen comprises type I α1, type I α2, type II α1, type III α1, type V α2, type VI α2, type VI 3, type VIII α1, type IX α2, type X α1, type XI α1, type XI α2, type XII α2, type XIV α1, and/or procollagen α1(V) collagen chains. In some embodiments, the bone-specific ECM comprises proteoglycans including aggrecan core protein, asporin, decorin, fibromodullin, heparan sulfate proteoglycan 2, lumican, osteoglycin/mimecan, osteomodulin, and/or proline/arginine-rich end leucine-rich repeat protein. In some embodiments, the bone-specific ECM comprises glycoproteins including AE binding protein 1, alpha-2-HS-glycoprotein, bone gamma-carboxyglutamate protein, biglycan, ECM protein 2, elastin, fibrillin 1, fibrinogen beta chain, fibrinogen gamma chain, fibronectin 1, periostin, osteonectin, transforming growth factor-beta-induced protein, thrombospondin 1, tenascin C, tenascin N, and/or vitronectin. In some embodiments, the bone-specific ECM comprises matrix-associated factors including albumin, annexin A2, acidic chitinase, creatine kinase B, mucin 5AC (oligomeric mucus/gel-forming) and/or collectin subfamily member 12 (collectin-12). In some embodiments, the bone-specific ECM comprises other structural factors including actin γ2 and/or vimentin. In some embodiments, the bone-specific ECM comprises ECM regulators including prothrombin, coagulation factor IX, coagulation factor X, inter-alpha (globulin) inhibitor H4, and/or serpin peptidase inhibitor, clade F. In some embodiments, the bone-specific ECM comprises matrisome-secreted factors including olfactomedin. In some embodiments, the bone-specific ECM comprises immune factors including complement component 3 (C3) and/or immunoglobulin G heavy chain. In some embodiments, the bone-specific ECM comprises marrow-associated factors including hemoglobin subunit α and/or hemoglobin subunit β.

In some embodiments, lung-specific ECM may comprise about 400-530 μg/mL collagens, about 40-50 μg/mL elastins, and about 3-5 μg/mL glycosaminoglycans. In some embodiments, the lung-specific ECM has an elastic modulus of about 3.1 kPa. However, the elastic modulus may be about 3 to about 6 kPa, about 2 to about 8 kPa, about 2 to about 12 kPa, or individual values or ranges therebetween. In some embodiments, the elastic modulus may be similar to the elastic modulus of natural lung tissue. In some embodiments, the collagen comprises type I α1, type I α2, type II α1, type III α1, type IV α1, type IV α2, type IV α3, type IV α4, type IV α5, type V α2, type VI α2, type VI α3, type VI α5, type VIII α1, type IX α2, type XI α1, type XI α2, type XVI α1, and/or procollagen α1(V) collagen chains. In some embodiments, the lung-specific ECM comprises proteoglycans including hyaluronan, heparan sulfate, aggrecan core protein, hyaluronan and proteoglycan link protein 1, and/or heparan sulfate proteoglycan 2. In some embodiments, the lung-specific ECM comprises glycoproteins including dermatopontin, elastin, fibrillin 1, fibulin 5, laminin γ1, laminin subunit α (e.g., α5), laminin subunit β (e.g., β2), microfibril associated protein 4, nidogen 1, and/or periostin. In some embodiments, the lung-specific ECM comprises matrix-associated factors including albumin and/or acidic chitinase. In some embodiments, the lung-specific ECM comprises other structural factors including actin γ2 and/or aquaporin-1. In some embodiments, the lung-specific ECM comprises matrisome-secreted factors including hornerin.

In some embodiments, liver-specific ECM may comprise about 1100-1300 μg/mL collagens, about 120-150 μg/mL elastins, and about 5-15 μg/mL glycosaminoglycans. In some embodiments, the liver-specific ECM has an elastic modulus of about 2.8 kPa. However, the elastic modulus may be about 2 to about 7 kPa, about 2 to about 10 kPa, about 2 to about 15 kPa, about 7 to about 15 kPa, or individual values or ranges therebetween. In some embodiments, the elastic modulus may be similar to the elastic modulus of natural liver tissue. In some embodiments, the collagen comprises type I α1, type I α2, type II α1, type III α1, type IV α2, type V α2, type VI α3, and type VI α5 collagen chains. In some embodiments, the liver-specific ECM comprises proteoglycans including heparan sulfate and/or heparan sulfate proteoglycan 2. In some embodiments, the liver-specific ECM comprises glycoproteins including EGF-contained fibulin-like ECM protein, elastin, fibrillin 1, fibrillin 2, laminin γ1, saposin-B-val, prostate stem cell antigen, and/or von Willebrand factor. In some embodiments, the liver-specific ECM comprises matrix-associated factors including albumin, acidic chitinase, mucin 5AC (oligomeric mucus/gel-forming), collectin-12, mucin 6 (oligomeric mucus/gel-forming), and/or trefoil factor 2. In some embodiments, the liver-specific ECM comprises other structural factors including actin, keratin type II cytoskeletal 1, keratin type I cytoskeletal 10, keratin type II cytoskeletal 2 epidermal, keratin type I cytoskeletal 9, myosin heavy chain 9, and/or tubulin beta chain. In some embodiments, the liver-specific ECM comprises ECM regulators including granulin precursor.

The composition of bone-specific ECM, lung-specific ECM, and liver-specific ECM are summarized in Table 1. However, these compositions are exemplary in nature and the TS-ECM profiles may vary therefrom as to any number of components.

TABLE 1
Summary of Mass Spectrometry Analyses of Tissue-Specific ECM Products.

| | | | TissueSpec ® ECM | | |
|---|---|---|---|---|---|
| Protein category | Protein | Gene | Bone | Liver | Lung |
| Collagens | type I alpha 1 chain | COL1A1 | ● | ● | ● |
| | type I alpha 2 chain | COL1A2 | ● | ● | ● |
| | type II alpha 1 chain | COL2A1 | ● | ● | ● |
| | type III alpha 1 chain | COL3A1 | ● | ● | ● |
| | type IV alpha 1 chain | COL4A1 | | | ● |
| | type IV alpha 2 chain | COL4A2 | | ● | ● |
| | type IV alpha 3 chain | COL4A3 | | | ● |
| | type IV alpha 4 chain | COL4A4 | | | ● |
| | type IV alpha 5 chain | COL4A5 | | | ● |
| | type V alpha 2 chain | COL5A2 | ● | ● | ● |
| | type VI alpha 2 chain | COL6A2 | ● | | ● |
| | type VI alpha 3 chain | COL6A3 | ● | ● | ● |
| | type VI alpha 5 chain | COL6A5 | | ● | |
| | type VIII alpha 1 chain | COL8A1 | ● | | ● |
| | type IX alpha 2 chain | COL9A2 | ● | | ● |
| | type X alpha 1 chain | COL10A1 | ● | | |
| | type XI alpha 1 chain | COL11A1 | ● | | ● |
| | type XI alpha 2 chain | COL11A2 | ● | | ● |
| | type XII alpha 2 chain | COL12A1 | ● | | |
| | type XIV alpha 1 chain | COL14A1 | ● | | |
| | type XVI alpha 1 chain | COL16A1 | | | ● |
| | procollagen alpha 1(V) | COL5A1 | ● | | ● |
| Proteoglycans | aggrecan core protein | ACAN | ● | | ● |
| | asporin | ASPN | ● | | |
| | decorin | DCN | ● | | |
| | fibromodulin | FMOD | ● | | |
| | hyaluronan and proteoglycan link protein 1 | HAPLN1 | | | ● |
| | heparan sulfate proteoglycan 2 | HSPG2 | ● | ● | ● |
| | lumican | LUM | ● | | |
| | osteoglycin/mimecan | OGN | ● | | |
| | osteomodulin | OMD | ● | | |
| | proline/arginine-rich end leucine-rich | PRELP | ● | | |
| Glycoproteins | AE binding protein 1 | AEBP1 | ● | | |
| | alpha-2-HS-glycoprotein | AHSG | ● | | |
| | bone gamma-carboxyglutamate protein | BGLAP | ● | | |
| | biglycan | BGN | ● | | |
| | dermatopontin | DPT | | | ● |
| | extracellular matrix protein 2 | ECM2 | ● | | |
| | EGF-containing fibulin-like ECM protein | EFEMP1 | | ● | |
| | elastin | ELN | ● | ● | ● |
| | fibrillin 1 | FBN1 | ● | ● | ● |
| | fibrillin 2 | FBN2 | | ● | |
| | fibulin 5 | FBLN5 | | | ● |
| | fibrinogen beta chain | FGB | ● | | |
| | fibrinogen gamma chain | FGG | ● | | |
| | fibronectin 1 | FN1 | ● | | |
| | laminin, gamma 1 | LAMC1 | | | ● |
| | laminin subunit alpha | LAMA5 | | | ● |
| | laminin subunit beta | LAMB2 | | | ● |
| | laminin, gamma 1 | LAMC1 | | | ● |
| | microfibril associated protein 4 | MFAP4 | | | ● |
| | nidogen 1 | NID1 | | | ● |
| | periostin | POSTN | ● | | ● |
| | saposin-B-val | PSAP | | ● | |
| | prostate stem cell antigen | PSCA | | ● | |
| | osteonectin | SPARC | ● | | |
| | transforming growth factor, beta induced | TGFBI | ● | | |
| | thrombospondin 1 | THBS1 | ● | | |
| | tenascin C | TNC | ● | | |
| | tenascin N | TNN | ● | | |
| | vitronectin | VTN | ● | | |
| | von Willebrand factor | VWF | | ● | |
| Matrix-associated | albumin | ALB | ● | ● | ● |
| factors | annexin A2 | ANXA2 | ● | | |
| | chitinase, acidic | CHIA | ● | ● | ● |
| | creatine kinase B | CKB | ● | | |
| | mucin 5AC collectin subfamily member 12 | MUC5AC | ● | | |
| | mucin 5AC oligomeric mucus/gel-forming | MUC5AC | | ● | |
| | mucin 6, oligomeric mucus/gel-forming | MUC6 | | ● | |
| | trefoil factor 2 | TFF2 | | ● | |
| Other structural factors | actin | ACTB | | ● | |
| | actin, gamma 2 | ACTG2 | ● | | ● |
| | aquaporin-1 | AQP1 | | | ● |
| | keratin, type II cytoskeletal 1 | KRT1 | | ● | |
| | keratin, type I cytoskeletal 10 | KRT10 | | ● | |
| | keratin, type II cytoskeletal 2 epidermal | KRT2 | | ● | |
| | keratin, type I cytoskeletal 9 | KRT9 | | ● | |
| | myosin heavy chain 9 | MYH9 | | ● | |
| | tubulin beta chain | TUBB | | ● | |
| | vimentin | VIM | ● | | |
| ECM regulators | prothrombin | F2 | ● | | |
| | coagulation factor IX | F9 | ● | | |
| | coagulation factor X | F10 | ● | | |
| | granulin precursor | GRN | | ● | |
| | inter-alpha (globulin) inhibitor H4 | ITIH4 | ● | | |
| | serpin peptidase inhibitor, clade F | SERPINF1 | ● | | |
| Matrisome-secreted | hornerin | HRNR | | | ● |
| factors | olfactomedin | OLFML3 | ● | | |
| Immune factors | complement C3 | C3 | ● | | |
| | immunoglobulin G heavy chain | IGHG | ● | | |
| Marrow-associated | hemoglobin subunit alpha | HBA | ● | | |
| factors | hemoglobin subunit beta | HBB | ● | | |

As described above, ECM comprises macromolecules (e.g., proteins, lipids, and polysaccharides) and other factors that are specific for cell-signaling in a particular niche-environment. In native ECM, the ECM components form a three-dimensional ultrastructure. In some applications, one may prefer a more uniform substrate like a solution or a hydrogel for culturing and modeling. The TS-ECM produced by such the methods described herein is distinct from native ECM. The TS-ECM is decellularized and the removal of the cellular structure modulates the concentrations of macromolecules and other cell-signaling factors. Further, the three-dimensional ultrastructure may be removed and the various components of the ECM may be digested into fragments. Any of the ECM components described herein may be fragmented in the TS-ECM, included but not limited to collagen, elastin, glycosaminoglycans, proteoglycans, matrix associated factors, ECM regulators, matrisome secreted factors, immune factors, marrow associated factors, and other structural factors. The removal of the three-dimensional ultrastructure of the ECM and the fragmentation of ECM components facilitates formation of a homogenous mixture for use in forming substrates such as hydrogels, surface coatings, fibers (e.g., electrospun fibers), liquid solution, media supplement, and bio-ink (e.g., printable bio-ink). Surprisingly, the fragmented components nonetheless contribute to cell signaling along with small molecules, thus retaining the characteristics of the niche environment to a high degree despite the fragmentation and lack of ultrastructure which are needed to form the conventional substrate structure.

In some embodiments, the TS-ECM is decellularized. In some embodiments, the decellularized TS-ECM comprises a homogenous mixture of macromolecule fragments including collagen, elastin, and glycosaminoglycan. In some embodiments, the decellularized TS-ECM comprises macromolecules including collagen, elastin, and glycosaminoglycan, wherein the amount of each macromolecule may be decreased after decellularization. In some embodiments, the decellularized TS-ECM comprises a homogenous mixture of macromolecule fragments including collagen, elastin, and glycosaminoglycan, wherein the concentration of each macromolecule may be changed after decellularization.

In some embodiments, the decellularized TS-ECM comprises a homogenous mixture of macromolecule fragments. In some embodiments, the decellularized TS-ECM comprises a homogenous mixture of macromolecule fragments, wherein the macromolecules may be fully or partially fragmented after enzymatic digestion. In some embodiments, the decellularized TS-ECM comprises a homogenous mixture of macromolecule fragments, wherein the homogenous mixture retains cellular signaling. In some embodiments, the decellularized TS-ECM comprises a homogenous mixture of macromolecule fragments, wherein the homogenous mixture does not contain the ECM three-dimensional ultra-structure. In some embodiments, the ECM three-dimensional ultra-structure is not required for cell-matrix recognition. In some embodiments, interactions responsible for cell-matrix recognition is not limited to structural cues from decellularized matrix, but also relies on signaling from small molecules or protein fragments. In some embodiments described herein, the decellularized TS-EMC is processed into an ECM powder. In some embodiments, the ECM powder comprises a homogenous mixture of macromolecule fragments, wherein the macromolecules may be fully or partially fragmented after enzymatic digestion. In some embodiments, the ECM powder comprises a homogenous mixture of macromolecule fragments, wherein the homogenous mixture retains cellular signaling. In some embodiments, the ECM powder comprises a homogenous mixture of macromolecule fragments, wherein the homogenous mixture does not contain the ECM three-dimensional ultra-structure. In some embodiments, the ECM three-dimensional ultra-structure is not required for cell-matrix recognition. In some embodiments, interactions responsible for cell-matrix recognition is not limited to structural cue from decellularized matrix, but also relies on signaling from small molecules or protein fragments.

In some embodiments, the TS-ECM may not be enzymatically digested and the three-dimensional ultrastructure may be maintained, e.g., as an acellular and/or dehydrated scaffold.

In some embodiments, the substrates may further include additional components beyond the TS-ECM components. In some embodiments, the substrates may include cell culture media, media supplements, or components thereof. In some embodiments, the substrates may include one or more of amino acids, glucose, salts, vitamins, carbohydrates, proteins, peptides, trace elements, other nutrients, extracts, additives, gases, or organic compounds. Additional components for the proper growth, maintenance and/or modeling of cells as would be known to one having an ordinary level of skill in the art are also contemplated herein.

The cell culture platform may be utilized with a variety of cancer cells types. In many cases, the cancer cells are of a type that is known to commonly metastasize. In some embodiments, the cancer cells are breast cancer cells. In some embodiments, the cancer cells are lung cancer cells. In some embodiments, the cancer cells are prostate cancer cells. In some embodiments, the cancer cells are colon cancer cells. In some embodiments, the cancer cells are rectal cancer cells. Additional types of cancer cells as would be known to one having an ordinary level of skill in the art are also contemplated herein. Further, the methods described herein can be performed with one or more cancer cell subtypes. For example, where the cancer cells are breast cancer cells, the cancer cells may include one or more of luminal A cells, luminal B cells, HER-2 enriched cells, and basal-like cells.

In some embodiments, the selected cancer cells may be foreign to at least one of the utilized TS-ECM substrates. In other words, the cancer cells are utilized with a TS-ECM substrate of a type to which the cells are not native, thus forming a metastatic colony. For example, breast cancer cells may be cultured in one or more of bone-specific ECM, lung-specific ECM, and liver-specific ECM. As such, the resulting colony will be a metastatic colony in that the cancer cells are native to a different niche environment (i.e., breast-specific ECM). However, in some embodiments, the selected cancer cells may be native to the utilized TS-ECM substrate resulting in a culture that models a primary cancer colony, e.g., an originating tumor site. For example, breast cancer cells may be cultured in breast-specific ECM such that the resulting colony is a primary breast cancer colony. In some embodiments, the selected cancer cells may be modeled in both native and foreign TS-ECM substrates. For example, culturing breast cancer cells in breast-specific ECM and one or more of bone-specific ECM, lung-specific ECM, and liver-specific ECM may highlight differences between the primary cancer and the metastatic cancer in a quantifiable manner.

The cell culture platform may be utilized with cancer cells from a variety of sources. In some embodiments, the cancer cells may be primary tumor cells or tumor-associated cells procured from a human or animal subject. In some embodiments, the cancer cells may be primary tumor cells or tumor-associated cells procured from a prospective patient in order to perform patient-specific therapy evaluation. By culturing cancer cells procured from the patient, various tumor-associated responses in the cell culture may exhibit a greater degree of similarity to the patient's cancer, thus increasing the value of the cell culture as a tool for evaluating the patient's cancer and planning treatment.

In additional embodiments, the cancer cells are procured from a cancer cell line. The cancer cells may be sourced from a variety of cancer cell lines. In some embodiments, the cancer cells are BT-549 breast cancer cells. In some embodiments, the cancer cells are T-47D breast cancer cells. In additional embodiments where breast cancer cells are utilized, the cancer cells may be 600 MPE cells, AMJ13 cells, AU565 cells, BT-20 cells, BT-474 cells, BT-483 cells, Evsa-T cells, Hs 578T cells, MCF7 cells, MDA-MB-231 cells, MDA-MB-468 cells, SkBr3 cells, or ZR-75-1 cells. In some embodiments, the cancer cells are adenocarcinoma A549 lung cancer cells. In some embodiments, the cancer cells are Jacket lung cancer cells. In additional embodiments where lung cancer cells are utilized, the cancer cells may be EKVX cells, HOP-62 cells, HOP-92 cells, NCI-H226 cells, NCI-H23 cells, NCI-H322M cells, NCI-H460 cells, NCI-H522 cells, PC9 cell, L068 cells, LUDLU-1 cells, COR-L105 cells, SKLU1 cells, SKMES1 cells, NCI-H727 cells, LC-2/AD cells, NCIH358 cells, ChaGo-K-1 cells, MOR/CPR cells, MOR/0.4R cells, or MOR/0.2R cells. In some embodiments, the cancer cells are prostate cancer cells, such as DU-145 cells or PC-3 cells. In some embodiments, the cancer cells are colon cancer cells, such as Colo205 cells, HCC-2998 cells, HCT-116 cells, HCT-15 cells, HT29 cells, KM12 cells, or SW-620 cells. Additional types of cancer cells and additional cancer cell lines are additionally contemplated herein, as would be known to a person having an ordinary level of skill in the art. Further, any combination of cancer cell types and/or cancer cell lines could be utilized with the cell culture platform.

The cell culture platform may further be configured, adapted, made and/or used in any manner described herein with respect to the method of making the cell culture platform, the kit for forming a cell culture platform, and the method of using the cell culture platform.

### Kit for Forming a Cell Culture Platform

In another aspect of the present subject matter, a kit forming a cell culture platform is provided. The kit includes at least one substrate precursor and at least one reagent. Each substrate precursor comprises a different decellularized tissue-specific extracellular matrix in a form configured to be converted into a TS-ECM substrate. The reagent is adapted to convert the precursor into a TS-ECM substrate. As discussed herein, in some embodiments the kit includes two or more substrate precursors to form multiple different TS-ECM substrates to emulate multiple different niche environments.

In some embodiments, the decellularized TS-ECM is selected from bone-specific ECM, lung-specific ECM, and liver-specific ECM. In additional embodiments, the TS-ECM may be selected from additional niche environments, such as brain-specific ECM, kidney-specific extracellular matrix, skin-specific extracellular matrix, intestine-specific extracellular matrix, heart-specific extracellular matrix, and lymph-specific extracellular matrix. In still additional embodiments, the TS-ECM may emulate a niche environment specific to another tissue. For example, the tissue may be selected from the adrenal gland, amnion, bladder, blood vessel, breast, cartilage, chorion, connective tissue, esophagus, eye, fat, larynx, ligament, microvasculature, muscle, mouth, omentum, ovary, fallopian tube, thyroid, parathyroid, large intestine, small intestine, pancreas, peritoneum, pharynx, placenta membrane, prostate, rectum, smooth muscle, spinal cord, spinal fluid, spleen, stomach, tendon, testes, thymus, umbilical cord, uterus, vagina, or Wharton's Jelly. In some embodiments, the TS-ECM may emulate a region of the anatomy, an organ, or a region of an organ. For example, left and right lungs have unique anatomies and may represent unique TS-ECMs which may be utilized individually or together for direct comparison. In another example, a TS-ECM may represent the large intestine or it may more specifically represent the colon or the rectum.

In some embodiments the kit comprises a plurality of substrate precursors. Each substrate precursor may comprise a different decellularized TS-ECM in order to emulate multiple niche environments with a single kit. For example, a kit may include one or more first precursors comprising a first TS-ECM, one or more second precursors comprising a second TS-ECM, and one or more third precursors comprising a third TS-ECM. In some embodiments, the first TS-ECM comprises bone-specific ECM, the second TS-ECM comprises lung-specific ECM, and the third TS-ECM comprises liver-specific ECM. However, any combination of TS-ECMs disclosed herein is contemplated. While a combination of three different TS-ECMs is demonstrated, it should be understood that other quantities are contemplated. A kit may comprise precursors for one, two, three, four, five, or more different TS-ECMs. In some embodiments, the reagent comprises one or more of a neutral buffer, a basic buffer, a base, and an acid. For example, a neutral buffer may comprise Phosphate Buffered Saline (PBS), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), and/or MOPS (3-(N-morpholino)propanesulfonic acid). For example, a basic buffer may comprise carbonate bicarbonate, TAPS ([tris(hydroxymethyl)methylamino]propanesulfonic acid), Bicine (2-(bis(2-hydroxyethyl)amino)acetic acid), Tris (tris(hydroxymethyl)aminomethane), and/or Tricine (N-[tris(hydroxymethyl)methyl]glycine). For example, a base may comprise Sodium Hydroxide (NaOH). For example, an acid may comprise Hydrochloric Acid (HCl) or Acetic Acid. In additional embodiments, the reagent may comprise deionized water. However, additional or alternative reagents may be provided to convert the precursor into various forms, as would be known to a person having an ordinary level of skill in the art. In still additional embodiments, a reagent is not required. As such, it may not be provided with the kit. Even further, where a reagent is required, in some embodiments the reagent may nonetheless not be provided with the kit. Rather, the kit may include instructions or indications related to the reagent to be utilized with the substrate precursor. A user may obtain the reagent and utilize it with the kit. For example, a kit may include a substrate precursor and instructions that instruct the user to add deionized water as a reagent. The instructions are described in greater detail below.

The substrate precursor may be provided in a variety of forms. For example, the substrate precursor may be selected from a solution, a dry foam, an intact scaffold, and a dry powder. Additionally, the reagent may be selected to convert the substrate precursor to any of a variety of substrate formats. In some embodiments, the reagent is configured to reconstitute the precursor into a hydrogel. For example, the precursor may comprise a solution and the reagent may comprise a base and a neutral buffer configured to convert the solution into a hydrogel. In another example, the precursor may comprise a dry foam (e.g., a dehydrated or “instant” hydrogel) and the reagent may comprise deionized water and/or a neutral buffer (e.g., PBS, HEPES, and/or TES). In some embodiments, the reagent is configured to reconstitute the precursor into a scaffold. For example, the precursor may comprise a dehydrated scaffold and the reagent may comprise deionized water and/or a neutral buffer (e.g., PBS) configured to rehydrate the scaffold. In another example, the precursor may comprise an intact (hydrated) scaffold and no reagent may be required. In some embodiments, the reagent is configured to solubilize the precursor into a surface coating. For example, the precursor may comprise a solution and the reagent may comprise a basic buffer and/or a neutral buffer configured to convert the solution into a surface coating. In some embodiments, the reagent is configured to convert the precursor into a bio-ink additive. For example, the precursor may comprise a dry powder and the reagent may comprise an acid configured to convert the dry powder into a bio-ink additive. In some embodiments, the reagent is configured to convert the precursor into a media supplement or other liquid solution. For example, the precursor may comprise an acidic solution and the reagent may comprise a neutral buffer (e.g., PBS) configured to neutralize the solution to form a media supplement. In another example, the reagent may comprise a neutral or basic solution and no reagent may be required.

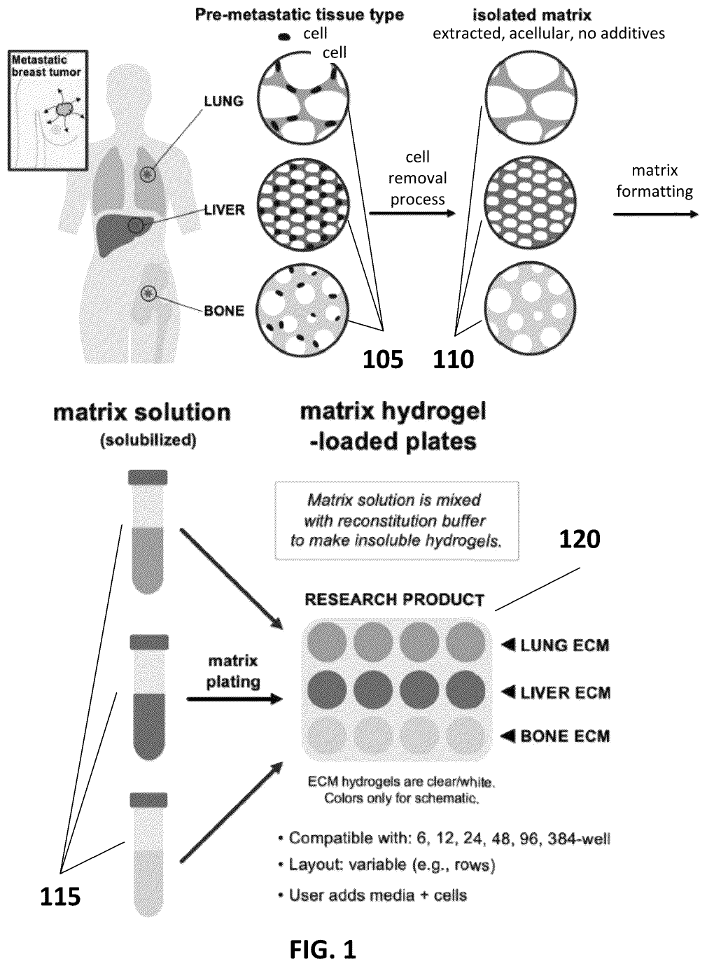

In some embodiments, the one or more precursors of the kit may be prepared by performing the steps of providing 105 one or more non-metastatic tissues, processing 110 the tissue to isolate TS-ECM, and solubilizing 115 the TS-ECM to produce matrix precursors. These steps are more fully described with respect to the method of making a cell culture platform as described herein and depicted in FIG. 1.

An exemplary embodiment of the disclosed method of forming the precursors of the kit is described in more detail herein with respect to kidney tissue. However, it is understood that the methods could be adapted for various tissues and employed in a similar manner to produce other tissue-specific cell culture platforms. According to an exemplary method, kidneys are procured and immediately frozen and prepared for sectioning. Frozen blocks are then sectioned longitudinally into thin (200 μm-1 mm) slices showing the entire cross-section of the kidney. The cortex, medulla, and papillae of the kidney are then dissected and separated from the thin slices prior to decellularization. The tissues are decellularized using a 4-step method consisting of 0.02% trypsin (2 hr.), 3% Tween-20 (2 hr.), 4% sodium deoxycholate (2 hr.), and 0.1% peracetic acid (1 hr.). Each step is followed by deionized water and 2×PBS washes. In some embodiments, each region is decellularized by serial washes in 0.02% trypsin, 3% Tween, 4% deoxycholic acid, and 0.1% peracetic acid solutions followed by enzymatic digestions. Following decellularization, the ECMs are snap frozen in liquid nitrogen, pulverized using a mortar and pestle, and then lyophilized to obtain a fine powder. Lyophilized ECM powder is digested using pepsin and hydrochloric acid for 48 hours at room temperature. The resulting digest is re-constituted into a hydrogel by increasing the ionic strength and the pH of the solution using Phosphate Buffered Saline (PBS) and Sodium Hydroxide (NaOH). The re-constituted hydrogel may be plated on a cell culture vessel (e.g., a well plate) to form the tissue-specific cell culture platform.

The described process may be adapted for various other tissues described herein. Tissue sections are decellularized by the introduction of one or more of deionized water, hypertonic salines, enzymes, detergents, and acids. In an exemplary embodiment, lobar liver sections are decellularized by 0.02% trypsin (120 min), 0.5% Ethylenediaminetetraacetic acid (EDTA)(30 min), 3% Tween-20, (120 min), 8 mM 3-[(3-cholamindoproyl)dimethylammonio]-1-propanesulfonate (CHAPS)(120 min). Each step is followed by deionized water and hypertonic (2×) phosphate-buffered saline (PBS) washes. Exemplary embodiments for various organs and tissues of human and animal origin are provided in Table 2.

TABLE 2

| Organ | Step 1 | Step 2 | Step 3 | Step 4 | Step 5 | Step 6 | Step 7 |
|---|---|---|---|---|---|---|---|
| Bladder | Trypsin | EDTA | Tween-20 | EDTA | Sodium | DNase I | Paracetic |
| | 0.02% | 0.5% | 3% | 0.5% | Deoxycholate | 0.2 | Acid 0.1% |
| | 120 min | 30 min | 120 min | 30 min | 4% | mg/mL | 30 min |
| | | | | | 120 min | 120 min | |
| Bone | 0.5M HCl | Tween-20 | EDTA | CHAPS | — | — | — |
| | 12 hours | 3% | 0.5% | 8 mM | | | |
| | | 120 min | 30 min | 120 min | | | |
| Esophagus | Trypsin | EDTA | Tween-20 | EDTA | Sodium | DNase I | Paracetic |
| (mucosa) | 0.02% | 0.5% | 3% | 0.5% | Deoxycholate | 0.2 | Acid 0.1% |
| | 120 min | 30 min | 120 min | 30 min | 4% | mg/mL | 30 min |
| | | | | | 120 min | 120 min | |
| Esophagus | Trypsin | EDTA | Trypsin | EDTA | Sodium | DNase I | Paracetic |
| (Smooth | 0.02% | 0.5% | 0.02% | 0.5% | Deoxycholate | 0.2 | Acid 0.1% |
| muscle) | 360 min | 30 min | 360 min | 30 min | 4% | mg/mL | 30 min |
| | | | | | 360 min | 120 min | |
| Heart | Trypsin | EDTA | Tween-20 | EDTA | Sodium | DNase I | Paracetic |
| | 0.02% | 0.5% | 3% | 0.5% | Deoxycholate | 0.2 | Acid 0.1% |
| | 60 min | 30 min | 120 min | 30 min | 4% | mg/mL | 30 min |
| | | | | | 120 min | 120 min | |
| Kidney | Trypsin | EDTA | Tween-20 | EDTA | Sodium | DNase I | Paracetic |
| | 0.02% | 0.5% | 3% | 0.5% | Deoxycholate | 0.2 | Acid 0.1% |
| | 120 min | 30 min | 120 min | 30 min | 4% | mg/mL | 30 min |
| | | | | | 120 min | 120 min | |
| Liver | 0.5M HCl | Tween-20 | EDTA | CHAPS | | | |
| | 120 min | 3% | 0.5% | 8 mM | | | |
| | | 20 min | 120 min | 120 min | | | |
| Lung | Trypsin | EDTA | Tween-20 | EDTA | Sodium | DNase I | Paracetic |
| | 0.02% | 0.5% | 3% | 0.5% | Deoxycholate | 0.2 | Acid 0.1% |
| | 120 min | 30 min | 120 min | 30 min | 4% | mg/mL | 30 min |
| | | | | | 120 min | 120 min | |
| Muscle | Trypsin | EDTA | Tween-20 | EDTA | Sodium | DNase I | Paracetic |
| | 0.02% | 0.5% | 3% | 0.5% | Deoxycholate | 0.2 | Acid 0.1% |
| | 60 min | 30 min | 120 min | 30 min | 4% | mg/mL | 30 min |
| | | | | | 120 min | 120 min | |

Following decellularization, resulting materials are terminally sterilized and biopsied according to desired scaffold size. In some embodiments, the scaffold is sized to fit in a cell culture vessel such as the wells of a standard microtiter plate, for example a 6-, 12-, 24-, 48-, or 96-well plate.

In some embodiments, following decellularization, an ECM solution is produced. The decellularized material is snap frozen in liquid nitrogen, pulverized using a mortar and pestle, milled, and lyophilized to obtain a fine ECM powder. In some embodiments, the ECM powder is digested using 1 mg/mL pepsin and 0.1 M hydrochloric acid for more than 1 hour at room temperature. The resulting digest is neutralized, frozen, and thawed to obtain ECM solution, i.e., the substrate precursor. However, the substrate precursor may be provided in other formats as described herein. In some embodiments, the ECM powder may be the substrate precursor. In other embodiments, the ECM powder may be additionally or alternatively processed into one of the other precursor formats described herein. In some embodiments, the kit further comprises instructions for utilizing the kit to produce the cell culture platform described herein. The instructions may comprise written or printed instructions, images, graphics, symbols, video files, audio files, links or directions for accessing any of the aforementioned, and combinations thereof. In some embodiments, the instructions include instructions for utilizing the precursor to reconstitute the precursor to a specified format. In some embodiments, the instructions include instructions for plating the reconstituted TS-ECM on a cell culture vessel. In some embodiments, the instructions include instructions for applying the reagent to the precursor. For example, where a reagent is not included in kit, the instructions may include a type of reagent and an amount of reagent to be applied to the precursor. In some embodiments, the instructions comprise instructions for a user to carry out the reconstitution and plating 120 steps as depicted in FIG. 1 and described with respect thereto, thereby forming the cell culture platform. In some embodiments, the instructions comprise instructions for seeding cancer cells within the TS-ECM and/or instructions for culturing or proliferating the cancer cells within the TS-ECM to form a colony.

The TS-ECM precursors may be derived from a variety of non-metastatic tissue sources. In some embodiments, the tissue source is selected from a human source and an animal source. For example, the tissue may be porcine (i.e., sourced from a pig) or any other animal tissue known to have clinical relevance. In some embodiments, the tissue source is selected from fetal tissue, juvenile tissue, and adult tissue. In some embodiments, the tissue source is selected from healthy tissue, diseased tissue, transgenic tissue, or tissue having a specific disorder or health condition. For example, in some embodiments, the tissue source is fibrotic tissue (i.e., exhibiting tissue fibrosis). The resulting TS-ECM is representative of extracellular matrix from the tissue source, or more generally from tissue having the same relevant characteristics as the tissue source (e.g., juvenile human lung tissue will yield lung-specific ECM representative of a juvenile human's lung tissue).

In some embodiments, the kit may be utilized with a cell culture vessel. In some embodiments, the cell culture vessel comprises a tissue culture plate. In some embodiments, the cell culture vessel may be a petri dish or other dish. In some embodiments, the cell culture vessel comprises a flask. Additional types of cell culture vessel as would be known to one having an ordinary level of skill in the art are also contemplated herein. The cell culture vessel may comprise one or more divided regions to be utilized for individual cell culture substrates, such that each precursor of the kit may be reconstituted in a separate divided region. For example, a tissue culture plate may comprise one or more wells. In some embodiments, the plate comprises 1 well, 3 wells, 6 wells, 12 wells, 24 wells, 48 wells, 96 wells, 384 wells, greater than 384 wells, or any individual value or any range between any two values therein.

In some embodiments, the kit has a shelf life of 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, or any individual value or any range between any two values therein.

In some embodiments, the kit may be utilized to form a cell culture platform comprising a plurality of TS-ECM substrates. For example, the kit may be utilized with a culture plate having a plurality of divided regions (e.g., wells), where each TS-ECM substrate is reconstituted in a separate divided region. In some embodiments, the plurality of TS-ECM substrates may include a variety of different tissue-specific extracellular matrices in order to emulate multiple niche environments in a single platform. For example, the completed cell culture platform may include one or more first wells comprising a first TS-ECM substrate, one or more second wells comprising a second TS-ECM substrate, and one or more third wells comprising a third TS-ECM substrate. In some embodiments, the first TS-ECM substrate comprises bone-specific ECM, the second TS-ECM substrate comprises lung-specific ECM, and the third TS-ECM substrate comprises liver-specific ECM. However, any combination of TS-ECM substrates disclosed herein is contemplated. While a combination of three different TS-ECM substrates is demonstrated, it should be understood that other quantities are contemplated. A culture plate may comprise two, three, four, five, or more different TS-ECM substrates.

In some embodiments, combinations of TS-ECM substrates are selected based on common sites of metastasis for a particular tumor type. In some embodiments, a cell culture platform for modeling breast cancer cells comprises one or more different TS-ECM substrates, each emulating a niche environment selected from bone tissue, liver tissue, lung tissue, and brain tissue. In some embodiments, a cell culture platform for modeling lung cancer cells comprises one or more different TS-ECM substrates each emulating a niche environment selected from bone tissue, liver tissue, opposite lung tissue (e.g., where the cancer cells are from a left lung, the TS-ECM emulates right lung tissue), brain tissue, and adrenal gland tissue. In some embodiments, a cell culture platform for modeling liver cancer cells comprises one or more different TS-ECM substrates, each emulating a niche environment selected from lung tissue, kidney tissue, and lymph tissue (e.g., portal lymph nodes). In some embodiments, a cell culture platform for modeling bone cancer cells comprises a TS-ECM substrate emulating a niche environment specific to lung tissue. In some embodiments, a cell culture platform for modeling brain cancer cells comprises one or more different TS-ECM substrate, each emulating a niche environment selected from spinal cord tissue and spinal fluid.

In additional embodiments, a cell culture platform for modeling bladder cancer cells comprises one or more different TS-ECM substrates, each emulating a niche environment selected from bone tissue, liver tissue, and lung tissue. In some embodiments, a cell culture platform for modeling colon cancer cells and/or rectal cancer cells comprises one or more different TS-ECM substrates, each emulating a niche environment selected from liver tissue, lung tissue, and peritoneal tissue. In some embodiments, a cell culture platform for modeling esophageal cancer cells comprises one or more different TS-ECM substrates, each emulating a niche environment selected from bone tissue, liver tissue, lung tissue, lymph node tissue, and stomach tissue. In some embodiments, a cell culture platform for modeling fallopian tube cancer cells comprises one or more different TS-ECM substrates, each emulating a niche environment selected from bone tissue, liver tissue, lung tissue, kidney tissue, brain tissue, peritoneal tissue, ovarian tissue, and uterine tissue. In some embodiments, a cell culture platform for modeling gallbladder cancer cells comprises one or more different TS-ECM substrates, each emulating a niche environment selected from liver tissue, pancreatic tissue, and lymph node tissue. In some embodiments, a cell culture platform for modeling kidney cancer cells comprises one or more different TS-ECM substrates, each emulating a niche environment selected from bone tissue, liver tissue, lung tissue, brain tissue, adrenal gland tissue, ovarian tissue, and testicular tissue. In some embodiments, a cell culture platform for modeling blood or bone marrow cancer cells (i.e., leukemia) comprises one or more different TS-ECM substrates, each emulating a niche environment selected from liver tissue, spleen tissue, spinal fluid, lymph node tissue, and testicular tissue. In some embodiments, a cell culture platform for modeling mouth cancer cells comprises one or more different TS-ECM substrates, each emulating a niche environment selected from lung tissue and lymph tissue (e.g., neck lymph nodes). In some embodiments, a cell culture platform for modeling oral and/or oropharyngeal cancer cells comprises one or more different TS-ECM substrates, each emulating a niche environment selected from lung tissue, kidney tissue, neck tissue, throat tissue, and prostate tissue. In some embodiments, a cell culture platform for modeling ovarian cancer cells comprises one or more different TS-ECM substrates, each emulating a niche environment selected from liver tissue, lung tissue, spleen tissue, peritoneal tissue, and fallopian tube tissue. In some embodiments, a cell culture platform for modeling pancreatic cancer cells comprises one or more different TS-ECM substrates, each emulating a niche environment selected from liver tissue, lung tissue, and peritoneal tissue. In some embodiments, a cell culture platform for modeling prostate cancer cells comprises one or more different TS-ECM substrates, each emulating a niche environment selected from bone tissue, liver tissue, lung tissue, and adrenal gland tissue. In some embodiments, a cell culture platform for modeling skin cancer cells comprises one or more different TS-ECM substrates, each emulating a niche environment selected from bone tissue, liver tissue, lung tissue, brain tissue, skin tissue, and muscular tissue. In some embodiments, a cell culture platform for modeling stomach cancer cells comprises one or more different TS-ECM substrates, each emulating a niche environment selected from liver tissue, lung tissue, and peritoneal tissue. In some embodiments, a cell culture platform for modeling testicular cancer cells comprises one or more different TS-ECM substrates, each emulating a niche environment selected from liver tissue and lymph node tissue. In some embodiments, a cell culture platform for modeling throat cancer cells comprises one or more different TS-ECM substrates, each emulating a niche environment selected from bone tissue and lung tissue. In some embodiments, a cell culture platform for modeling thyroid cancer cells comprises one or more different TS-ECM substrates, each emulating a niche environment selected from bone tissue, liver tissue, and lung tissue. In some embodiments, a cell culture platform for modeling urethral cancer cells comprises one or more different TS-ECM substrates, each emulating a niche environment selected from bone tissue, liver tissue, lung tissue, kidney tissue, and lymph node tissue. In some embodiments, a cell culture platform for modeling uterine cancer cells comprises one or more different TS-ECM substrates, each emulating a niche environment selected from bone tissue, liver tissue, lung tissue, peritoneal tissue, rectal tissue, bladder tissue, fallopian tube tissue, and vaginal tissue.

In still additional embodiments, a cell culture platform for modeling non-Hodgkin lymphoma cells comprises one or more different TS-ECM substrates, each emulating a niche environment selected from bone tissue, liver tissue, and lung tissue. In some embodiments, a cell culture platform for modeling multiple myeloma cells comprises one or more different TS-ECM substrates, each emulating a niche environment selected from central nervous system tissue (e.g., brain, spinal cord, spinal fluid) and blood. In some embodiments, a cell culture platform for modeling neuroblastoma cells comprises one or more different TS-ECM substrates, each emulating a niche environment selected from liver tissue and adrenal gland tissue. In some embodiments, a cell culture platform for modeling ocular melanoma cells comprises one or more different TS-ECM substrates, each emulating a niche environment selected from bone tissue, liver tissue, and lung tissue. In some embodiments, a cell culture platform for modeling sarcoma cells comprises a TS-ECM substrate emulating a niche environment specific to lung tissue. Additional types of cancer cells and/or additional sets of TS-ECM substrates are contemplated herein as would be known to one having an ordinary level of skill in the art.

In some embodiments of the cell culture platform formed by the kit described herein, each TS-ECM substrate of the cell culture platform is segregated, i.e., completely physically separated from other TS-ECM substrates. The physical separation must be capable of preventing cell transfer between the TS-ECM substrates, co-mingling of cell culture components, interaction, cross-contamination, or any other influence of one substrate or culture upon another. In some embodiments, the segregation comprises a barrier such as a wall between the TS-ECM substrates. For example, as described, a tissue culture plate with a plurality of wells may be utilized such that the walls of the wells serve as a physical barrier between the TS-ECMs. Other types of barriers may be utilized as would be known to one having an ordinary level of skill in the art. In some embodiments, an adequate amount of physical spacing between TS-ECM substrates may provide sufficient segregation. For example, as described above, a tissue culture plate may include divided regions which are adequately spaced to provide for individual TS-ECM substrates. Further, in some embodiments, multiple plates or vessels may be utilized, where one or more TS-ECMs are provided on each plate or vessel in order to provide segregation. Various additional manners of providing physical separation between substrates as would be known to one having an ordinary level of skill in the art are contemplated herein.

In additional embodiments, each TS-ECM substrate may be compartmentalized, i.e., physically separated from the other TS-ECM substrates to prevent intermixing in a manner that would substantially alter the composition of any of the TS-ECM substrates. Compartmentalized TS-ECM substrates may include a means of fluid communication therebetween. For example, the compartmentalization may allow for some cell transfer, interaction, or other influence of one substrate or culture upon another (e.g., transfer of some molecules or creation of a gradient therebetween). In some embodiments, the TS-ECM substrates may be housed in physically separated compartments as described above (e.g., connected vessels, connected chambers of a vessel, etc.) except with fluid channels extending between the compartments. In some embodiments, the compartments comprise microfluidic chambers on a vessel such as chip (e.g., an organ-on-a-chip system). In some embodiments, each compartment comprises a printed bio-ink in a region of a vessel such as a chip. Further, the fluid communication between compartments may be formed in a variety of manners. In some embodiments, the compartments communicate via interconnecting channels spanning between the compartments. For example, the channels may be microfluidic channels. In some embodiments, the compartments are separated by a porous membrane that allows fluid communication therebetween. The fluid communication may be configured to allow transport of fluids, molecules, cells, or a combination thereof. Additionally, the fluid communication may be arranged in a variety of manners. In some embodiments, each of the additional compartments directly fluidly communicate with the first compartment in parallel circuit arrangement. For example, the compartments may be arranged in a hub-and-spoke arrangement where the first compartment serves as a central hub having direct fluid communication with each of the radially arranged additional compartments (i.e., spokes). However, the same structural connectivity may be formed with different physical arrangements. In additional embodiments, the first compartment and the additional compartments directly communicate in a series circuit arrangement (i.e., arranged in a chain) such that some additional compartments indirectly communicate with the first compartment (i.e., fluid communication occurs through a directly communicating compartment). Combinations of parallel and series connections are also contemplated herein. In some embodiments, at least one of the additional compartments directly communicate with the first compartment while the remaining additional compartments indirectly communicate with the first compartment. Several layers of interconnectivity may be formed in this manner. In some embodiments, the interconnectivity may mimic a biological system. For example, the TS-ECMs and the interconnectivity therebetween may mimic the interconnectivity of parts of an organ, a plurality of organs, and/or an organ system.

The TS-ECM has a specified composition that emulates the ECM found in a specific native tissue. As such, the composition of each TS-ECM may vary. Each TS-ECM may comprise a different combination of proteoglycans, collagens, elastins, multiadhesive proteins, hyaluronic acid, CAMs, and additional components. Each of these components may have subtypes, the presence of each of which may vary from one TS-ECM to another TS-ECM. Each TS-ECM may be characterized by the presence or absence of one or more components. Further, the concentration of each component may vary from one TS-ECM to another TS-ECM. These variations result in each TS-ECM having unique physical characteristics, such as architecture and stiffness, and unique cell interaction characteristics, such as gene expression, ECM remodeling, and cell proliferation.

In some embodiments, bone-specific ECM may comprise about 580-620 μg/mL collagens, about 40-50 μg/mL elastins, and about 10-20 μg/mL glycosaminoglycans. In some embodiments, the bone-specific ECM has an elastic modulus of about 6.6 kPa. However, the elastic modulus may be about 6 to about 25 kPa, about 6 to about 10⁵ kPa, or individual values or ranges therebetween. In some embodiments, the elastic modulus may be similar to the elastic modulus of natural bone tissue. In some embodiments, the collagen comprises type I α1, type I α2, type II α1, type III α1, type V α2, type VI α2, type VI α3, type VIII α1, type IX α2, type X α1, type XI α1, type XI α2, type XII α2, type XIV α1, and/or procollagen α1(V) collagen chains. In some embodiments, the bone-specific ECM comprises proteoglycans including aggrecan core protein, asporin, decorin, fibromodullin, heparan sulfate proteoglycan 2, lumican, osteoglycin/mimecan, osteomodulin, and/or proline/arginine-rich end leucine-rich repeat protein. In some embodiments, the bone-specific ECM comprises glycoproteins including AE binding protein 1, alpha-2-HS-glycoprotein, bone gamma-carboxyglutamate protein, biglycan, ECM protein 2, elastin, fibrillin 1, fibrinogen beta chain, fibrinogen gamma chain, fibronectin 1, periostin, osteonectin, transforming growth factor-beta-induced protein, thrombospondin 1, tenascin C, tenascin N, and/or vitronectin. In some embodiments, the bone-specific ECM comprises matrix-associated factors including albumin, annexin A2, acidic chitinase, creatine kinase B, mucin 5AC (oligomeric mucus/gel-forming) and/or collectin subfamily member 12 (collectin-12). In some embodiments, the bone-specific ECM comprises other structural factors including actin γ2 and/or vimentin. In some embodiments, the bone-specific ECM comprises ECM regulators including prothrombin, coagulation factor IX, coagulation factor X, inter-alpha (globulin) inhibitor H4, and/or serpin peptidase inhibitor, clade F. In some embodiments, the bone-specific ECM comprises matrisome-secreted factors including olfactomedin. In some embodiments, the bone-specific ECM comprises immune factors including complement component 3 (C3) and/or immunoglobulin G heavy chain. In some embodiments, the bone-specific ECM comprises marrow-associated factors including hemoglobin subunit α and/or hemoglobin subunit β.

In some embodiments, lung-specific ECM may comprise about 400-530 μg/mL collagens, about 40-50 μg/mL elastins, and about 3-5 μg/mL glycosaminoglycans. In some embodiments, the lung-specific ECM has an elastic modulus of about 3.1 kPa. However, the elastic modulus may be about 3 to about 6 kPa, about 2 to about 8 kPa, about 2 to about 12 kPa, or individual values or ranges therebetween. In some embodiments, the elastic modulus may be similar to the elastic modulus of natural lung tissue. In some embodiments, the collagen comprises type I α1, type I α2, type II α1, type III α1, type IV α1, type IV α2, type IV α3, type IV α4, type IV α5, type V α2, type VI α2, type VI α3, type VI α5, type VIII α1, type IX α2, type XI α1, type XI α2, type XVI α1, and/or procollagen α1(V) collagen chains. In some embodiments, the lung-specific ECM comprises proteoglycans including hyaluronan, heparan sulfate, aggrecan core protein, hyaluronan and proteoglycan link protein 1, and/or heparan sulfate proteoglycan 2. In some embodiments, the lung-specific ECM comprises glycoproteins including dermatopontin, elastin, fibrillin 1, fibulin 5, laminin γ1, laminin subunit α (e.g., α5), laminin subunit β (e.g., β2), microfibril associated protein 4, nidogen 1, and/or periostin. In some embodiments, the lung-specific ECM comprises matrix-associated factors including albumin and/or acidic chitinase. In some embodiments, the lung-specific ECM comprises other structural factors including actin γ2 and/or aquaporin-1. In some embodiments, the lung-specific ECM comprises matrisome-secreted factors including hornerin.

In some embodiments, liver-specific ECM may comprise about 1100-1300 μg/mL collagens, about 120-150 μg/mL elastins, and about 5-15 μg/mL glycosaminoglycans. In some embodiments, the liver-specific ECM has an elastic modulus of about 2.8 kPa. However, the elastic modulus may be about 2 to about 7 kPa, about 2 to about 10 kPa, about 2 to about 15 kPa, about 7 to about 15 kPa, or individual values or ranges therebetween. In some embodiments, the elastic modulus may be similar to the elastic modulus of natural liver tissue. In some embodiments, the collagen comprises type I α1, type I α2, type II α1, type III α1, type IV α2, type V α2, type VI α3, and type VI α5 collagen chains. In some embodiments, the liver-specific ECM comprises proteoglycans including heparan sulfate and/or heparan sulfate proteoglycan 2. In some embodiments, the liver-specific ECM comprises glycoproteins including EGF-contained fibulin-like ECM protein, elastin, fibrillin 1, fibrillin 2, laminin γ1, saposin-B-val, prostate stem cell antigen, and/or von Willebrand factor. In some embodiments, the liver-specific ECM comprises matrix-associated factors including albumin, acidic chitinase, mucin 5AC (oligomeric mucus/gel-forming), collectin-12, mucin 6 (oligomeric mucus/gel-forming), and/or trefoil factor 2. In some embodiments, the liver-specific ECM comprises other structural factors including actin, keratin type II cytoskeletal 1, keratin type I cytoskeletal 10, keratin type II cytoskeletal 2 epidermal, keratin type I cytoskeletal 9, myosin heavy chain 9, and/or tubulin beta chain. In some embodiments, the liver-specific ECM comprises ECM regulators including granulin precursor.

The composition of bone-specific ECM, lung-specific ECM, and liver-specific ECM are summarized in Table 1. However, these compositions are exemplary in nature and the TS-ECM profiles may vary therefrom as to any number of components.

In some embodiments, the precursors and/or the substrates formed therewith may further include additional components beyond the TS-ECM components. In some embodiments, the precursors and/or substrates may include cell culture media, media supplements, or components thereof. In some embodiments, the precursors and/or substrates may include one or more of amino acids, glucose, salts, vitamins, carbohydrates, proteins, peptides, trace elements, other nutrients, extracts, additives, gases, or organic compounds. Additional components for the proper growth, maintenance and/or modeling of cells as would be known to one having an ordinary level of skill in the art are also contemplated herein.

As described above, ECM comprises macromolecules (e.g. proteins, lipids, and polysaccharides) and other factors that are specific for cell-signaling in a particular niche-environment. In native ECM, the ECM components form a three-dimensional ultrastructure. In some applications, one may prefer a more uniform substrate like a solution or a hydrogel for culturing and modeling. The TS-ECM produced by such the methods described herein is distinct from native ECM. The TS-ECM is decellularized and the removal of the cellular structure modulates the concentrations of macromolecules and other cell-signaling factors. Further, the three-dimensional ultrastructure may be digested into fragments. Any ECM components described herein may be fragmented in the TS-ECM, included but not limited to collagen, elastin, glycosaminoglycans, proteoglycans, matrix associated factors, ECM regulators, matrisome secreted factors, immune factors, marrow associated factors, and other structural factors. The removal of the three-dimensional ultrastructure of the ECM and the fragmentation of ECM components facilitates formation of a homogenous mixture for use in forming substrates such as hydrogels, surface coatings, fibers (e.g., electrospun fibers), liquid solution, media supplement, and bio-ink (e.g., printable bio-ink). Surprisingly, the fragmented components nonetheless contribute to cell signaling along with small molecules, thus retaining the characteristics of the niche environment to a high degree despite the fragmentation and lack of ultrastructure which are needed to form the conventional substrate structure.

In some embodiments, the composition comprises decellularized TS-ECM comprising a homogenous mixture of macromolecule fragments including collagen, elastin, and glycosaminoglycan. In some embodiments, the composition comprises decellularized TS-ECM comprising macromolecules including collagen, elastin, and glycosaminoglycan, wherein the amount of each macromolecule may be decreased after decellularization. In some embodiments, the composition comprises decellularized TS-ECM comprising a homogenous mixture of macromolecule fragments including collagen, elastin, and glycosaminoglycan, wherein the concentration of each macromolecule may be changed after decellularization.

In some embodiments, the composition comprises decellularized TS-ECM comprising a homogenous mixture of macromolecule fragments. In some embodiments, the decellularized TS-ECM comprises a homogenous mixture of macromolecule fragments, wherein the macromolecules may be fully or partially fragmented after enzymatic digestion. In some embodiments, the decellularized TS-ECM comprises a homogenous mixture of macromolecule fragments, wherein the homogenous mixture retains cellular signaling. In some embodiments, the decellularized TS-ECM comprises a homogenous mixture of macromolecule fragments, wherein the homogenous mixture does not contain the ECM three-dimensional ultra-structure. In some embodiments, the ECM three-dimensional ultra-structure is not required for cell-matrix recognition. In some embodiments, interactions responsible for cell-matrix recognition is not limited to structural cues from decellularized matrix, but also relies on signaling from small molecules or protein fragments. In some embodiments described herein, the decellularized TS-EMC is processed into an ECM powder. In some embodiments, the ECM powder comprises a homogenous mixture of macromolecule fragments, wherein the macromolecules may be fully or partially fragmented after enzymatic digestion. In some embodiments, the ECM powder comprises a homogenous mixture of macromolecule fragments, wherein the homogenous mixture retains cellular signaling. In some embodiments, the ECM powder comprises a homogenous mixture of macromolecule fragments, wherein the homogenous mixture does not contain the ECM three-dimensional ultra-structure. In some embodiments, the ECM three-dimensional ultra-structure is not required for cell-matrix recognition. In some embodiments, interactions responsible for cell-matrix recognition is not limited to structural cue from decellularized matrix, but also relies on signaling from small molecules or protein fragments.

In some embodiments, the TS-ECM may not be enzymatically digested and the three-dimensional ultrastructure may be maintained, e.g., as an acellular and/or dehydrated scaffold.

The kit and the resulting cell culture platform may be utilized with a variety of cancer cells types. In many cases, the cancer cells are of a type that is known to commonly metastasize. In some embodiments, the cancer cells are breast cancer cells. In some embodiments, the cancer cells are lung cancer cells. In some embodiments, the cancer cells are prostate cancer cells. In some embodiments, the cancer cells are colon cancer cells. In some embodiments, the cancer cells are rectal cancer cells. Additional types of cancer cells as would be known to one having an ordinary level of skill in the art are also contemplated herein. Further, the methods described herein can be performed with one or more cancer cell subtypes. For example, where the cancer cells are breast cancer cells, the cancer cells may include one or more of luminal A cells, luminal B cells, HER-2 enriched cells, and basal-like cells.

In some embodiments, the selected cancer cells may be foreign to at least one of the utilized TS-ECM substrates. In other words, the cancer cells are utilized with a TS-ECM substrate of a type to which the cells are not native, thus forming a metastatic colony. For example, breast cancer cells may be cultured in one or more of bone-specific ECM, lung-specific ECM, and liver-specific ECM. As such, the resulting colony will be a metastatic colony in that the cancer cells are native to a different niche environment (i.e., breast-specific ECM). However, in some embodiments, the selected cancer cells may be native to the utilized TS-ECM substrate resulting in a culture that models a primary cancer colony, e.g., an originating tumor site. For example, breast cancer cells may be cultured in breast-specific ECM such that the resulting colony is a primary breast cancer colony. In some embodiments, the selected cancer cells may be modeled in both native and foreign TS-ECM substrates. For example, culturing breast cancer cells in breast-specific ECM and one or more of bone-specific ECM, lung-specific ECM, and liver-specific ECM may highlight differences between the primary cancer and the metastatic cancer in a quantifiable manner.

The kit and the resulting cell culture platform may be utilized with cancer cells from a variety of sources. In some embodiments, the cancer cells may be primary tumor cells or tumor-associated cells procured from a human or animal subject. In some embodiments, the cancer cells may be primary tumor cells or tumor-associated cells procured from a prospective patient in order to perform patient-specific therapy evaluation. By culturing cancer cells procured from the patient, various tumor-associated responses in the cell culture may exhibit a greater degree of similarity to the patient's cancer, thus increasing the value of the cell culture as a tool for evaluating the patient's cancer and planning treatment.

In additional embodiments, the cancer cells are procured from a cancer cell line. The cancer cells may be sourced from a variety of cancer cell lines. In some embodiments, the cancer cells are BT-549 breast cancer cells. In some embodiments, the cancer cells are T-47D breast cancer cells. In additional embodiments where breast cancer cells are utilized, the cancer cells may be 600 MPE cells, AMJ13 cells, AU565 cells, BT-20 cells, BT-474 cells, BT-483 cells, Evsa-T cells, Hs 578T cells, MCF7 cells, MDA-MB-231 cells, MDA-MB-468 cells, SkBr3 cells, or ZR-75-1 cells. In some embodiments, the cancer cells are adenocarcinoma A549 lung cancer cells. In some embodiments, the cancer cells are Jacket lung cancer cells. In additional embodiments where lung cancer cells are utilized, the cancer cells may be EKVX cells, HOP-62 cells, HOP-92 cells, NCI-H226 cells, NCI-H23 cells, NCI-H322M cells, NCI-H460 cells, NCI-H522 cells, PC9 cell, L068 cells, LUDLU-1 cells, COR-L105 cells, SKLU1 cells, SKMES1 cells, NCI-H727 cells, LC-2/AD cells, NCIH358 cells, ChaGo-K-1 cells, MOR/CPR cells, MOR/0.4R cells, or MOR/0.2R cells. In some embodiments, the cancer cells are prostate cancer cells, such as DU-145 cells or PC-3 cells. In some embodiments, the cancer cells are colon cancer cells, such as Colo205 cells, HCC-2998 cells, HCT-116 cells, HCT-15 cells, HT29 cells, KM12 cells, or SW-620 cells. Additional types of cancer cells and additional cancer cell lines are additionally contemplated herein, as would be known to a person having an ordinary level of skill in the art. Further, any combination of cancer cell types and/or cancer cell lines could be utilized with the cell culture platform.

The kit for forming a cell culture platform may further be configured, adapted, made and/or used in any manner described herein with respect to the cell culture platform, the method of making the cell culture platform, and the method of using the cell culture platform.

### Method of Making a Cell Culture Platform

In another aspect of the present subject matter, a method of making a cell culture platform for modeling metastatic cancer is provided. FIG. 1 depicts a diagram of an illustrative method of making a cell culture platform in accordance with an embodiment. As shown in FIG. 1, one or more non-metastatic tissues are provided 105 and each non-metastatic tissue is processed 110 to isolate a decellularized tissue-specific extracellular matrix. The decellularized TS-ECMs are solubilized 115 to produce matrix solutions and reconstituted and plated 120 on a cell culture vessel to form a cell culture platform having one or more TS-ECM substrates.

The non-metastatic tissues may be derived from a variety of tissue types. For example, as depicted in FIG. 1, the one or more non-metastatic tissues are selected from bone tissue, lung tissue, and liver tissue. In additional embodiments, the non-metastatic tissues may additionally or alternative selected from additional tissues described herein.

The resulting TS-ECM derived from each non-metastatic tissue will emulate the niche environment specific to that tissue. In some embodiments, the TS-ECM may emulate common sites of metastasis. For example, the TS-ECM may be selected from bone-specific ECM, lung-specific ECM, and liver-specific ECM. In additional embodiments, the TS-ECM may be selected from additional niche environments, such as brain-specific ECM, kidney-specific extracellular matrix, skin-specific extracellular matrix, intestine-specific extracellular matrix, heart-specific extracellular matrix, and lymph-specific extracellular matrix. In still additional embodiments, the TS-ECM may emulate a niche environment specific to another tissue. For example, the tissue may be selected from the adrenal gland, amnion, bladder, blood vessel, breast, cartilage, chorion, connective tissue, esophagus, eye, fat, larynx, ligament, microvasculature, muscle, mouth, omentum, ovary, fallopian tube, thyroid, parathyroid, large intestine, small intestine, pancreas, peritoneum, pharynx, placenta membrane, prostate, rectum, smooth muscle, spinal cord, spinal fluid, spleen, stomach, tendon, testes, thymus, umbilical cord, uterus, vagina, or Wharton's Jelly. In some embodiments, the TS-ECM may emulate a region of the anatomy, an organ, or a region of an organ. For example, left and right lungs have unique anatomies and may represent unique TS-ECMs which may be utilized individually or together for direct comparison. In another example, a TS-ECM may represent the large intestine or it may more specifically represent the colon or the rectum.

The non-metastatic tissues may be derived from a variety of tissue sources. In some embodiments, the tissue source is selected from a human source and an animal source. For example, the tissue may be porcine (i.e., sourced from a pig) or any other animal tissue known to have clinical relevance. In some embodiments, the tissue source is selected from fetal tissue, juvenile tissue, and adult tissue. In some embodiments, the tissue source is selected from healthy tissue, diseased tissue, transgenic tissue, or tissue having a specific disorder or health condition. For example, in some embodiments, the tissue source is fibrotic tissue (i.e., exhibiting tissue fibrosis). The resulting TS-ECM is representative of extracellular matrix from the tissue source, or more generally from tissue having the same relevant characteristics as the tissue source (e.g., juvenile human lung tissue will yield lung-specific ECM representative of a juvenile human's lung tissue). An exemplary embodiment of the disclosed method is described in more detail herein with respect to kidney tissue. However, it is understood that the methods could be adapted for various tissues and employed in a similar manner to produce other tissue-specific cell culture platforms. According to an exemplary method, kidneys are procured and immediately frozen and prepared for sectioning. Frozen blocks are then sectioned longitudinally into thin (200 μm-1 mm) slices showing the entire cross-section of the kidney. The cortex, medulla, and papillae of the kidney are then dissected and separated from the thin slices prior to decellularization. The tissues are decellularized using a 4-step method consisting of 0.02% trypsin (2 hr.), 3% Tween-20 (2 hr.), 4% sodium deoxycholate (2 hr.), and 0.1% peracetic acid (1 hr.). Each step is followed by deionized water and 2×PBS washes. In some embodiments, each region is decellularized by serial washes in 0.02% trypsin, 3% Tween, 4% deoxycholic acid, and 0.1% peracetic acid solutions followed by enzymatic digestions. Following decellularization, the ECMs are snap frozen in liquid nitrogen, pulverized using a mortar and pestle, and then lyophilized to obtain a fine powder. Lyophilized ECM powder is digested using pepsin and hydrochloric acid for 48 hours at room temperature. The resulting digest is re-constituted into a hydrogel by increasing the ionic strength and the pH of the solution using Phosphate Buffered Saline (PBS) and Sodium Hydroxide (NaOH). The re-constituted hydrogel may be plated on a cell culture vessel (e.g., a well plate) to form the tissue-specific cell culture platform.

The described process may be adapted for various other tissues described herein. Tissue sections are decellularized by the introduction of one or more of deionized water, hypertonic salines, enzymes, detergents, and acids. In an exemplary embodiment, lobar liver sections are decellularized by 0.02% trypsin (120 min), 0.5% Ethylenediaminetetraacetic acid (EDTA)(30 min), 3% Tween-20, (120 min), 8 mM 3-[(3-cholamindoproyl)dimethlammonio]-1-propanesulfonate (CHAPS)(120 min). Each step is followed by deionized water and hypertonic (2×) phosphate-buffered saline (PBS) washes. Exemplary embodiments for various organs and tissues of human and animal origin are provided in Table 2.

Following decellularization, resulting materials are terminally sterilized and biopsied according to desired scaffold size. In some embodiments, the scaffold is sized to fit in a cell culture vessel such as the wells of a standard microtiter plate, for example a 6-, 12-, 24-, 48-, or 96-well plate.

In some embodiments, following decellularization, an ECM solution is produced. The decellularized material is snap frozen in liquid nitrogen, pulverized using a mortar and pestle, milled, and lyophilized to obtain a fine ECM powder. In some embodiments, the ECM powder is digested using 1 mg/mL pepsin and 0.1 M hydrochloric acid for more than 1 hour at room temperature. The resulting digest is neutralized, frozen, and thawed to obtain ECM solution.

In some embodiments, ECM powder is further processed to form an ECM sponge. ECM powder is digested using 1 mg/mL pepsin and 0.1 M hydrochloric acid for less than 24 hours at room temperature. The resulting digest is subjected to repeated cycles of high-speed centrifugation (5,000 rpm) and vortexing. The resulting material is transferred to a mold of desired dimensions and lyophilized. The resulting sponge can be sectioned, re-sized, or rehydrated. In some embodiments, the sponge is sized to fit in the wells of a standard a microtiter plate, for example a 6-, 12-, 24-, 48-, or 96-well plate. In some embodiments, ECM solution is ECM solution is re-constituted into a hydrogel by increasing the ionic strength and the pH of the solution using PBS and NaOH.

In some embodiments, the cell culture vessel comprises a tissue culture plate. In some embodiments, the cell culture vessel may be a petri dish or other dish. In some embodiments, the cell culture vessel comprises a flask. Additional types of cell culture vessel as would be known to one having an ordinary level of skill in the art are also contemplated herein. The cell culture vessel may comprise one or more divided regions to be utilized for individual TS-ECM substrates. For example, a tissue culture plate may comprise one or more wells. In some embodiments, the plate comprises 1 well, 3 wells, 6 wells, 12 wells, 24 wells, 48 wells, 96 wells, 384 wells, greater than 384 wells, or any individual value or any range between any two values therein.

In some embodiments, the in vitro cell culture platform has a shelf life of about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, or any individual value or any range between any two values therein.

In some embodiments, the cell culture platform is formed with a plurality of TS-ECM substrates. For example, the method may include providing a culture plate having a plurality of divided regions (e.g., wells), where each region houses TS-ECM substrate. In some embodiments, the plurality of TS-ECM substrates may include a variety of different tissue-specific extracellular matrices in order to emulate multiple niche environments in a single platform. For example, a culture plate may include one or more first wells comprising a first TS-ECM substrate, one or more second wells comprising a second TS-ECM substrate, and one or more third wells comprising a third TS-ECM substrate. In some embodiments, the first TS-ECM substrate comprises bone-specific ECM, the second TS-ECM substrate comprises lung-specific ECM, and the third TS-ECM substrate comprises liver-specific ECM. However, any combination of TS-ECM substrates disclosed herein is contemplated. While a combination of three different TS-ECM substrates is demonstrated, it should be understood that other quantities are contemplated. A culture plate may comprise two, three, four, five, or more different TS-ECM substrates.

As described above, ECM comprises macromolecules (e.g., proteins, lipids, and polysaccharides) and other factors that are specific for cell-signaling in a particular niche-environment. In native ECM, the ECM components form a three-dimensional ultrastructure. In some applications, one may prefer a more uniform substrate like a solution or a hydrogel for culturing and modeling. The TS-ECM produced by such the methods described herein is distinct from native ECM. The TS-ECM is decellularized and the removal of the cellular structure modulates the concentrations of macromolecules and other cell-signaling factors. Further, the three-dimensional ultrastructure may be removed and the various components of the ECM may be digested into fragments. Any of the ECM components described herein may be fragmented in the TS-ECM, including but not limited to collagen, elastin, glycosaminoglycans, proteoglycans, matrix associated factors, ECM regulators, matrisome secreted factors, immune factors, marrow associated factors, and other structural factors. The removal of the three-dimensional ultrastructure of the ECM and the fragmentation of ECM components facilitates formation of a homogenous mixture for use in forming substrates such as hydrogels, surface coatings, fibers (e.g., electrospun fibers), liquid solution, media supplement, and bio-ink (e.g., printable bio-ink). Surprisingly, the fragmented components nonetheless contribute to cell signaling along with small molecules, thus retaining the characteristics of the niche environment to a high degree despite the fragmentation and lack of ultrastructure which are needed to form the conventional substrate structure.

In some embodiments, the method of making a cell culture platform comprises decellularized TS-ECM comprising a homogenous mixture of macromolecule fragments including collagen, elastin, and glycosaminoglycan. In some embodiments, the method of making a cell culture platform comprises decellularized TS-ECM comprising macromolecules including collagen, elastin, and glycosaminoglycan, wherein the amount of each macromolecule may be decreased after decellularization. In some embodiments, the method of making a cell culture platform comprises decellularized TS-ECM comprising a homogenous mixture of macromolecule fragments including collagen, elastin, and glycosaminoglycan, wherein the concentration of each macromolecule may be changed after decellularization.

In some embodiments, the method of making a cell culture platform comprises decellularized TS-ECM comprising a homogenous mixture of macromolecule fragments. In some embodiments, the decellularized TS-ECM comprises a homogenous mixture of macromolecule fragments, wherein the macromolecules may be fully or partially fragmented after enzymatic digestion. In some embodiments, the decellularized TS-ECM comprises a homogenous mixture of macromolecule fragments, wherein the homogenous mixture retains cellular signaling. In some embodiments, the decellularized TS-ECM comprises a homogenous mixture of macromolecule fragments, wherein the homogenous mixture does not contain the ECM three-dimensional ultra-structure. In some embodiments, the ECM three-dimensional ultra-structure is not required for cell-matrix recognition. In some embodiments, interactions responsible for cell-matrix recognition is not limited to structural cues from decellularized matrix, but also relies on signaling from small molecules or protein fragments. In some embodiments described herein, the decellularized TS-EMC is processed into an ECM powder. In some embodiments, the ECM powder comprises a homogenous mixture of macromolecule fragments, wherein the macromolecules may be fully or partially fragmented after enzymatic digestion. In some embodiments, the ECM powder comprises a homogenous mixture of macromolecule fragments, wherein the homogenous mixture retains cellular signaling. In some embodiments, the ECM powder comprises a homogenous mixture of macromolecule fragments, wherein the homogenous mixture does not contain the ECM three-dimensional ultra-structure. In some embodiments, the ECM three-dimensional ultra-structure is not required for cell-matrix recognition. In some embodiments, interactions responsible for cell-matrix recognition is not limited to structural cue from decellularized matrix, but also relies on signaling from small molecules or protein fragments.

In some embodiments, the TS-ECM may not be enzymatically digested and the three-dimensional ultrastructure may be maintained, e.g., as an acellular and/or dehydrated scaffold.

In some embodiments, combinations of TS-ECM substrates are selected based on common sites of metastasis for a particular tumor type. In some embodiments, a cell culture platform for modeling breast cancer cells comprises one or more different TS-ECM substrates, each emulating a niche environment selected from bone tissue, liver tissue, lung tissue, and brain tissue. In some embodiments, a cell culture platform for modeling lung cancer cells comprises one or more different TS-ECM substrates each emulating a niche environment selected from bone tissue, liver tissue, opposite lung tissue (e.g., where the cancer cells are from a left lung, the TS-ECM emulates right lung tissue), brain tissue, and adrenal gland tissue. In some embodiments, a cell culture platform for modeling liver cancer cells comprises one or more different TS-ECM substrates, each emulating a niche environment selected from lung tissue, kidney tissue, and lymph tissue (e.g., portal lymph nodes). In some embodiments, a cell culture platform for modeling bone cancer cells comprises a TS-ECM substrate emulating a niche environment specific to lung tissue. In some embodiments, a cell culture platform for modeling brain cancer cells comprises one or more different TS-ECM substrate, each emulating a niche environment selected from spinal cord tissue and spinal fluid.

In additional embodiments, a cell culture platform for modeling bladder cancer cells comprises one or more different TS-ECM substrates, each emulating a niche environment selected from bone tissue, liver tissue, and lung tissue. In some embodiments, a cell culture platform for modeling colon cancer cells and/or rectal cancer cells comprises one or more different TS-ECM substrates, each emulating a niche environment selected from liver tissue, lung tissue, and peritoneal tissue. In some embodiments, a cell culture platform for modeling esophageal cancer cells comprises one or more different TS-ECM substrates, each emulating a niche environment selected from bone tissue, liver tissue, lung tissue, lymph node tissue, and stomach tissue. In some embodiments, a cell culture platform for modeling fallopian tube cancer cells comprises one or more different TS-ECM substrates, each emulating a niche environment selected from bone tissue, liver tissue, lung tissue, kidney tissue, brain tissue, peritoneal tissue, ovarian tissue, and uterine tissue. In some embodiments, a cell culture platform for modeling gallbladder cancer cells comprises one or more different TS-ECM substrates, each emulating a niche environment selected from liver tissue, pancreatic tissue, and lymph node tissue. In some embodiments, a cell culture platform for modeling kidney cancer cells comprises one or more different TS-ECM substrates, each emulating a niche environment selected from bone tissue, liver tissue, lung tissue, brain tissue, adrenal gland tissue, ovarian tissue, and testicular tissue. In some embodiments, a cell culture platform for modeling blood or bone marrow cancer cells (i.e., leukemia) comprises one or more different TS-ECM substrates, each emulating a niche environment selected from liver tissue, spleen tissue, spinal fluid, lymph node tissue, and testicular tissue. In some embodiments, a cell culture platform for modeling mouth cancer cells comprises one or more different TS-ECM substrates, each emulating a niche environment selected from lung tissue and lymph tissue (e.g., neck lymph nodes). In some embodiments, a cell culture platform for modeling oral and/or oropharyngeal cancer cells comprises one or more different TS-ECM substrates, each emulating a niche environment selected from lung tissue, kidney tissue, neck tissue, throat tissue, and prostate tissue. In some embodiments, a cell culture platform for modeling ovarian cancer cells comprises one or more different TS-ECM substrates, each emulating a niche environment selected from liver tissue, lung tissue, spleen tissue, peritoneal tissue, and fallopian tube tissue. In some embodiments, a cell culture platform for modeling pancreatic cancer cells comprises one or more different TS-ECM substrates, each emulating a niche environment selected from liver tissue, lung tissue, and peritoneal tissue. In some embodiments, a cell culture platform for modeling prostate cancer cells comprises one or more different TS-ECM substrates, each emulating a niche environment selected from bone tissue, liver tissue, lung tissue, and adrenal gland tissue. In some embodiments, a cell culture platform for modeling skin cancer cells comprises one or more different TS-ECM substrates, each emulating a niche environment selected from bone tissue, liver tissue, lung tissue, brain tissue, skin tissue, and muscular tissue. In some embodiments, a cell culture platform for modeling stomach cancer cells comprises one or more different TS-ECM substrates, each emulating a niche environment selected from liver tissue, lung tissue, and peritoneal tissue. In some embodiments, a cell culture platform for modeling testicular cancer cells comprises one or more different TS-ECM substrates, each emulating a niche environment selected from liver tissue and lymph node tissue. In some embodiments, a cell culture platform for modeling throat cancer cells comprises one or more different TS-ECM substrates, each emulating a niche environment selected from bone tissue and lung tissue. In some embodiments, a cell culture platform for modeling thyroid cancer cells comprises one or more different TS-ECM substrates, each emulating a niche environment selected from bone tissue, liver tissue, and lung tissue. In some embodiments, a cell culture platform for modeling urethral cancer cells comprises one or more different TS-ECM substrates, each emulating a niche environment selected from bone tissue, liver tissue, lung tissue, kidney tissue, and lymph node tissue. In some embodiments, a cell culture platform for modeling uterine cancer cells comprises one or more different TS-ECM substrates, each emulating a niche environment selected from bone tissue, liver tissue, lung tissue, peritoneal tissue, rectal tissue, bladder tissue, fallopian tube tissue, and vaginal tissue.

In still additional embodiments, a cell culture platform for modeling non-Hodgkin lymphoma cells comprises one or more different TS-ECM substrates, each emulating a niche environment selected from bone tissue, liver tissue, and lung tissue. In some embodiments, a cell culture platform for modeling multiple myeloma cells comprises one or more different TS-ECM substrates, each emulating a niche environment selected from central nervous system tissue (e.g., brain, spinal cord, spinal fluid) and blood. In some embodiments, a cell culture platform for modeling neuroblastoma cells comprises one or more different TS-ECM substrates, each emulating a niche environment selected from liver tissue and adrenal gland tissue. In some embodiments, a cell culture platform for modeling ocular melanoma cells comprises one or more different TS-ECM substrates, each emulating a niche environment selected from bone tissue, liver tissue, and lung tissue. In some embodiments, a cell culture platform for modeling sarcoma cells comprises a TS-ECM substrate emulating a niche environment specific to lung tissue. Additional types of cancer cells and/or additional sets of TS-ECM substrates are contemplated herein as would be known to one having an ordinary level of skill in the art.

In some embodiments, each TS-ECM substrate of the cell culture platform is segregated, i.e., completely physically separated from other TS-ECM substrates. The physical separation must be capable of preventing cell transfer between the TS-ECM substrates, co-mingling of cell culture components, interaction, cross-contamination, or any other influence of one substrate or culture upon another. In some embodiments, the segregation comprises a barrier such as a wall between the TS-ECM substrates. For example, as described, a tissue culture plate with a plurality of wells may be utilized such that the walls of the wells serve as a physical barrier between the TS-ECMs. Other types of barriers may be utilized as would be known to one having an ordinary level of skill in the art. In some embodiments, an adequate amount of physical spacing between TS-ECM substrates may provide sufficient segregation. For example, as described above, a tissue culture plate may include divided regions which are adequately spaced to provide for individual TS-ECM substrates. Further, in some embodiments, multiple plates or vessels may be utilized, where one or more TS-ECMs are provided on each plate or vessel in order to provide segregation. Various additional manners of providing physical separation between substrates as would be known to one having an ordinary level of skill in the art are contemplated herein.

In additional embodiments, each TS-ECM substrate may be compartmentalized, i.e., physically separated from the other TS-ECM substrates to prevent intermixing in a manner that would substantially alter the composition of any of the TS-ECM substrates. Compartmentalized TS-ECM substrates may include a means of fluid communication therebetween. For example, the compartmentalization may allow for some cell transfer, interaction, or other influence of one substrate or culture upon another (e.g., transfer of some molecules or creation of a gradient therebetween). In some embodiments, the TS-ECM substrates may be housed in physically separated compartments as described above (e.g., connected vessels, connected chambers of a vessel, etc.) except with fluid channels extending between the compartments. In some embodiments, the compartments comprise microfluidic chambers on a vessel such as chip (e.g., an organ-on-a-chip system). In some embodiments, each compartment comprises a printed bio-ink in a region of a vessel such as a chip. Further, the fluid communication between compartments may be formed in a variety of manners. In some embodiments, the compartments communicate via interconnecting channels spanning between the compartments. For example, the channels may be microfluidic channels. In some embodiments, the compartments are separated by a porous membrane that allows fluid communication therebetween. The fluid communication may be configured to allow transport of fluids, molecules, cells, or a combination thereof. Additionally, the fluid communication may be arranged in a variety of manners. In some embodiments, each of the additional compartments directly fluidly communicate with the first compartment in parallel circuit arrangement. For example, the compartments may be arranged in a hub-and-spoke arrangement where the first compartment serves as a central hub having direct fluid communication with each of the radially arranged additional compartments (i.e., spokes). However, the same structural connectivity may be formed with different physical arrangements. In additional embodiments, the first compartment and the additional compartments directly communicate in a series circuit arrangement (i.e., arranged in a chain) such that some additional compartments indirectly communicate with the first compartment (i.e., fluid communication occurs through a directly communicating compartment). Combinations of parallel and series connections are also contemplated herein. In some embodiments, at least one of the additional compartments directly communicate with the first compartment while the remaining additional compartments indirectly communicate with the first compartment. Several layers of interconnectivity may be formed in this manner. In some embodiments, the interconnectivity may mimic a biological system. For example, the TS-ECMs and the interconnectivity therebetween may mimic the interconnectivity of parts of an organ, a plurality of organs, and/or an organ system.

The TS-ECM may be processed and provided in a variety of substrate formats. In some embodiments, the format of the TS-ECM substrate may be selected from a hydrogel, a scaffold (e.g., an acellular scaffold), a surface coating, a sponge, fibers (e.g., electrospun fibers), liquid solution, media supplement, and bio-ink (e.g., printable bio-ink).

The TS-ECM has a specified composition that emulates the ECM found in a specific native tissue. As such, the composition of each TS-ECM may vary. Each TS-ECM may comprise a different combination of proteoglycans, collagens, elastins, multiadhesive proteins, hyaluronic acid, CAMs, and additional components. Each of these components may have subtypes, the presence of each of which may vary from one TS-ECM to another TS-ECM. Each TS-ECM may be characterized by the presence or absence of one or more components. Further, the concentration of each component may vary from one TS-ECM to another TS-ECM. These variations result in each TS-ECM having unique physical characteristics, such as architecture and stiffness, and unique cell interaction characteristics, such as gene expression, ECM remodeling, and cell proliferation.

In some embodiments, bone-specific ECM may comprise about 580-620 μg/mL collagens, about 40-50 μg/mL elastins, and about 10-20 μg/mL glycosaminoglycans. In some embodiments, the bone-specific ECM has an elastic modulus of about 6.6 kPa. However, the elastic modulus may be about 6 to about 25 kPa, about 6 to about 10⁵ kPa, or individual values or ranges therebetween. In some embodiments, the elastic modulus may be similar to the elastic modulus of natural bone tissue. In some embodiments, the collagen comprises type I α1, type I α2, type II α1, type III α1, type V α2, type VI α2, type VI α3, type VIII α1, type IX α2, type X α1, type XI α1, type XI α2, type XII α2, type XIV α1, and/or procollagen α1(V) collagen chains. In some embodiments, the bone-specific ECM comprises proteoglycans including aggrecan core protein, asporin, decorin, fibromodullin, heparan sulfate proteoglycan 2, lumican, osteoglycin/mimecan, osteomodulin, and/or proline/arginine-rich end leucine-rich repeat protein. In some embodiments, the bone-specific ECM comprises glycoproteins including AE binding protein 1, alpha-2-HS-glycoprotein, bone gamma-carboxyglutamate protein, biglycan, ECM protein 2, elastin, fibrillin 1, fibrinogen beta chain, fibrinogen gamma chain, fibronectin 1, periostin, osteonectin, transforming growth factor-beta-induced protein, thrombospondin 1, tenascin C, tenascin N, and/or vitronectin. In some embodiments, the bone-specific ECM comprises matrix-associated factors including albumin, annexin A2, acidic chitinase, creatine kinase B, mucin 5AC (oligomeric mucus/gel-forming) and/or collectin subfamily member 12 (collectin-12). In some embodiments, the bone-specific ECM comprises other structural factors including actin γ2 and/or vimentin. In some embodiments, the bone-specific ECM comprises ECM regulators including prothrombin, coagulation factor IX, coagulation factor X, inter-alpha (globulin) inhibitor H4, and/or serpin peptidase inhibitor, clade F. In some embodiments, the bone-specific ECM comprises matrisome-secreted factors including olfactomedin. In some embodiments, the bone-specific ECM comprises immune factors including complement component 3 (C3) and/or immunoglobulin G heavy chain. In some embodiments, the bone-specific ECM comprises marrow-associated factors including hemoglobin subunit α and/or hemoglobin subunit β.

In some embodiments, lung-specific ECM may comprise about 400-530 μg/mL collagens, about 40-50 μg/mL elastins, and about 3-5 μg/mL glycosaminoglycans. In some embodiments, the lung-specific ECM has an elastic modulus of about 3.1 kPa. However, the elastic modulus may be about 3 to about 6 kPa, about 2 to about 8 kPa, about 2 to about 12 kPa, or individual values or ranges therebetween. In some embodiments, the elastic modulus may be similar to the elastic modulus of natural lung tissue. In some embodiments, the collagen comprises type I α1, type I α2, type II α1, type III α1, type IV α1, type IV α2, type IV α3, type IV α4, type IV α5, type V α2, type VI α2, type VI α3, type VI α5, type VIII α1, type IX α2, type XI α1, type XI α2, type XVI α1, and/or procollagen α1(V) collagen chains. In some embodiments, the lung-specific ECM comprises proteoglycans including hyaluronan, heparan sulfate, aggrecan core protein, hyaluronan and proteoglycan link protein 1, and/or heparan sulfate proteoglycan 2. In some embodiments, the lung-specific ECM comprises glycoproteins including dermatopontin, elastin, fibrillin 1, fibulin 5, laminin γ1, laminin subunit α (e.g., α5), laminin subunit β (e.g., β2), microfibril associated protein 4, nidogen 1, and/or periostin. In some embodiments, the lung-specific ECM comprises matrix-associated factors including albumin and/or acidic chitinase. In some embodiments, the lung-specific ECM comprises other structural factors including actin γ2 and/or aquaporin-1. In some embodiments, the lung-specific ECM comprises matrisome-secreted factors including hornerin.

In some embodiments, liver-specific ECM may comprise about 1100-1300 μg/mL collagens, about 120-150 μg/mL elastins, and about 5-15 μg/mL glycosaminoglycans. In some embodiments, the liver-specific ECM has an elastic modulus of about 2.8 kPa. However, the elastic modulus may be about 2 to about 7 kPa, about 2 to about 10 kPa, about 2 to about 15 kPa, about 7 to about 15 kPa, or individual values or ranges therebetween. In some embodiments, the elastic modulus may be similar to the elastic modulus of natural liver tissue. In some embodiments, the collagen comprises type I α1, type I α2, type II α1, type III α1, type IV α2, type V α2, type VI α3, and type VI α5 collagen chains. In some embodiments, the liver-specific ECM comprises proteoglycans including heparan sulfate and/or heparan sulfate proteoglycan 2. In some embodiments, the liver-specific ECM comprises glycoproteins including EGF-contained fibulin-like ECM protein, elastin, fibrillin 1, fibrillin 2, laminin γ1, saposin-B-val, prostate stem cell antigen, and/or von Willebrand factor. In some embodiments, the liver-specific ECM comprises matrix-associated factors including albumin, acidic chitinase, mucin 5AC (oligomeric mucus/gel-forming), collectin-12, mucin 6 (oligomeric mucus/gel-forming), and/or trefoil factor 2. In some embodiments, the liver-specific ECM comprises other structural factors including actin, keratin type II cytoskeletal 1, keratin type I cytoskeletal 10, keratin type II cytoskeletal 2 epidermal, keratin type I cytoskeletal 9, myosin heavy chain 9, and/or tubulin beta chain. In some embodiments, the liver-specific ECM comprises ECM regulators including granulin precursor.

The composition of bone-specific ECM, lung-specific ECM, and liver-specific ECM are summarized in Table 1. However, these compositions are exemplary in nature and the TS-ECM profiles may vary therefrom as to any number of components.

In some embodiments, the substrates may further include additional components beyond the TS-ECM components. In some embodiments, the substrates may include cell culture media, media supplements, or components thereof. In some embodiments, the substrates may include one or more of amino acids, glucose, salts, vitamins, carbohydrates, proteins, peptides, trace elements, other nutrients, extracts, additives, gases, or organic compounds. Additional components for the proper growth, maintenance and/or modeling of cells as would be known to one having an ordinary level of skill in the art are also contemplated herein.

The cell culture platform may be utilized with a variety of cancer cells types. In many cases, the cancer cells are of a type that is known to commonly metastasize. In some embodiments, the cancer cells are breast cancer cells. In some embodiments, the cancer cells are lung cancer cells. In some embodiments, the cancer cells are prostate cancer cells. In some embodiments, the cancer cells are colon cancer cells. In some embodiments, the cancer cells are rectal cancer cells. Additional types of cancer cells as would be known to one having an ordinary level of skill in the art are also contemplated herein. Further, the methods described herein can be performed with one or more cancer cell subtypes. For example, where the cancer cells are breast cancer cells, the cancer cells may include one or more of luminal A cells, luminal B cells, HER-2 enriched cells, and basal-like cells.

In some embodiments, the selected cancer cells may be foreign to at least one of the utilized TS-ECM substrates. In other words, the cancer cells are utilized with a TS-ECM substrate of a type to which the cells are not native. For example, breast cancer cells may be cultured in one or more of bone-specific ECM, lung-specific ECM, and liver-specific ECM. As such, the resulting colony will be a metastatic colony in that the cancer cells are native to a different niche environment (i.e., breast-specific ECM). However, in some embodiments, the selected cancer cells may be native to the utilized TS-ECM substrate resulting in a culture that models a primary cancer colony, e.g., an originating tumor site. For example, breast cancer cells may be cultured in breast-specific ECM such that the resulting colony is a primary breast cancer colony. In some embodiments, the selected cancer cells may be modeled in both native and foreign TS-ECM substrates. For example, culturing breast cancer cells in breast-specific ECM and one or more of bone-specific ECM, lung-specific ECM, and liver-specific ECM may highlight differences between the primary cancer and the metastatic cancer in a quantifiable manner.

The cell culture platform may be utilized with cancer cells from a variety of sources. In some embodiments, the cancer cells may be primary tumor cells or tumor-associated cells procured from a human or animal subject. In some embodiments, the cancer cells may be primary tumor cells or tumor-associated cells procured from a prospective patient in order to perform patient-specific therapy evaluation. By culturing cancer cells procured from the patient, various tumor-associated responses in the cell culture may exhibit a greater degree of similarity to the patient's cancer, thus increasing the value of the cell culture as a tool for evaluating the patient's cancer and planning treatment.

In additional embodiments, the cancer cells are procured from a cancer cell line. The cancer cells may be sourced from a variety of cancer cell lines. In some embodiments, the cancer cells are BT-549 breast cancer cells. In some embodiments, the cancer cells are T-47D breast cancer cells. In additional embodiments where breast cancer cells are utilized, the cancer cells may be 600 MPE cells, AMJ13 cells, AU565 cells, BT-20 cells, BT-474 cells, BT-483 cells, Evsa-T cells, Hs 578T cells, MCF7 cells, MDA-MB-231 cells, MDA-MB-468 cells, SkBr3 cells, or ZR-75-1 cells. In some embodiments, the cancer cells are adenocarcinoma A549 lung cancer cells. In some embodiments, the cancer cells are Jacket lung cancer cells. In additional embodiments where lung cancer cells are utilized, the cancer cells may be EKVX cells, HOP-62 cells, HOP-92 cells, NCI-H226 cells, NCI-H23 cells, NCI-H322M cells, NCI-H460 cells, NCI-H522 cells, PC9 cell, L068 cells, LUDLU-1 cells, COR-L105 cells, SKLU1 cells, SKMES1 cells, NCI-H727 cells, LC-2/AD cells, NCIH358 cells, ChaGo-K-1 cells, MOR/CPR cells, MOR/0.4R cells, or MOR/0.2R cells. In some embodiments, the cancer cells are prostate cancer cells, such as DU-145 cells or PC-3 cells. In some embodiments, the cancer cells are colon cancer cells, such as Colo205 cells, HCC-2998 cells, HCT-116 cells, HCT-15 cells, HT29 cells, KM12 cells, or SW-620 cells. Additional types of cancer cells and additional cancer cell lines are additionally contemplated herein, as would be known to a person having an ordinary level of skill in the art. Further, any combination of cancer cell types and/or cancer cell lines could be utilized with the cell culture platform.

The method of making a cell culture platform may further be adapted in any manner described herein with respect to the cell culture platform, the kit for forming a cell culture platform, and the method of using the cell culture platform.

### Methods of Using a Cell Culture Platform as Described Herein

In another aspect of the present invention, methods of using the cell culture platform are provided. Specifically, methods of modeling metastatic cancer with the cell culture platform are provided. In some embodiments, the method comprises providing one or more different TS-ECM substrates, such as the cell culture platform described herein. The method may further comprise culturing cancer cells in the TS-ECM substrates, wherein the cancer cells are foreign to at least one of the TS-ECM substrates. In some embodiments, culturing cancer cells comprises seeding cancer cells within the TS-ECM substrates, and proliferating the cancer cells to form colonies. The method may further comprise assessing at least one tumor-associated response of the colonies. As discussed herein, in some embodiments the comprised providing two or more different TS-ECM substrates to form colonies in multiple different niche environments.

In some embodiments, the tumor-associated response comprises a metabolism of the cancer cells and/or colony. For example, cancer cell metabolism may be measured and compared over time. In some embodiments, the tumor-associated response comprises cell motility. For example, the movement of cancer cells may be measured and recorded at one or several points in time. In some embodiments, the tumor-associated response comprises cell viability. For example, survival rate or death rate of the cancer cells may be approximated at one or several points in time. In some embodiments, the tumor-associated response comprises proliferation of the cancer cells and/or colony. For example, proliferation or proliferation rate may be measured and compared over time. In some embodiments, the tumor-associated response comprises ECM remodeling. For example, changes in mechanical stiffness (elastic modulus) in response to cancer cell presence may be evaluated. In some embodiments, the tumor-associated response comprises gene expression and/or regulation of tumor-associated genes. For example, expression of tumor-associated genes by the cancer cells in the TS-ECM may be evaluated by measuring RNA expression. In some embodiments, the tumor-associated response comprises protein expression and/or regulation of protein-encoding genes. For example, expression of proteins-coding genes may be evaluated by measuring RNA expression of a specific protein-encoding gene and/or assessing the presence and concentration of the specific protein.

In some embodiments, the method may further comprise applying a therapy to the colony. In some embodiments, applying a therapy to the colony comprises contacting the colony with a drug. In other embodiments, applying a therapy to the colony comprises applying radiation or other therapies as would be known to one having an ordinary level of skill in the art. In such embodiments, the tumor-associated response may comprise evaluating the therapy in order to determine efficacy. In some embodiments, the therapy may be a known cancer therapy (e.g., drugs such as Fulvestrant, Palbociclib, Paclitaxel, Erlotinib, Etoposide, KPT-185, and/or Tivantinib). In other embodiments, the therapy may be a potential cancer therapy.

In embodiments where the method includes applying a therapy to the colony, the tumor-associated response may comprise cell viability. For example, cell viability may be evaluated in samples where incremental amounts/concentrations of a drug are applied to the colony in order to evaluate drug efficacy. The efficacy for each amount/concentration of the drug may be compared in order to determine effective doses.

In the method described herein, the cancer cells are foreign to at least one of the TS-ECM substrates. As such, culturing the cancer cells therein results in the formation of a metastatic colony. In some embodiments a plurality of TS-ECM substrates to which the cancer cells are foreign may be utilized, thereby resulting in the formation of a plurality of different types of metastatic colonies (e.g. metastatic breast cancer in bone ECM, metastatic breast cancer in lung ECM, metastatic breast cancer in liver ECM, etc.). In some embodiments, where two or more different TS-ECM substrates are utilized, one of the TS-ECM substrates may be the native TS-ECM substrate of the cancer cells. For example, where breast cancer cells are utilized, one of the TS-ECM substrates may comprise breast-specific ECM. As such, culturing the cancer cells therein results in the formation of a primary cancer colony.

In another aspect of the present invention, additional methods of using the cell culture platform are provided. Specifically, methods of modeling cancer cell invasion (i.e., the process of a primary cancer metastasizing to another tissue) with the cell culture platform are provided. In some embodiments, the method comprises providing a plurality of compartments in fluid communication with one another, wherein each compartment houses a different TS-ECM substrate in the manner described herein. The method further comprises culturing cancer cells as described herein in a first compartment of the plurality of compartments. The method may further comprise assessing at least one tumor-associated response of the colony.

In some embodiments, the tumor-associated response comprises cell motility and/or cell migration. For example, the movement of cancer cells from the first compartment to any additional compartments may be measured and recorded at one or several points in time (e.g., a cell invasion assay). In some embodiments, the presence of the cancer cells in additional compartments may be observed. In some embodiments, additional measurements or observations may be made to evaluate cell motility and/or cell migration. For example, proliferation of the cancer cells in the additional compartments may be quantified. The assessment may further comprise evaluating any of the tumor-associated responses described herein for the first compartment and/or the additional compartments. For example, the method may include assessing metabolism, cell viability, cell proliferation, ECM remodeling, gene expression and/or regulation of tumor-associated genes, protein expression and/or regulation of protein-encoding genes, and/or drug efficacy for the first compartment and/or the additional compartments.

The compartments may be formed in a variety of manners. In some embodiments, the compartments comprise separate connected vessels, such as plates or flasks. In some embodiments, the compartments comprise separate compartments on a single vessel, such as wells on a plate. In some embodiments, the compartments comprise microfluidic chambers on a vessel such as chip (e.g., an organ-on-a-chip system). In some embodiments, each compartment comprises a printed bio-ink in a region of a vessel such as a chip. Further, the fluid communication between compartments may be formed in a variety of manners. In some embodiments, the compartments communicate via interconnecting channels spanning between the compartments. For example, the channels may be microfluidic channels. In some embodiments, the compartments are separated by a porous membrane that allows fluid communication therebetween. The fluid communication may be configured to allow transport of fluids, molecules, cells, or a combination thereof. Additionally, the fluid communication may be arranged in a variety of manners. In some embodiments, each of the additional compartments directly fluidly communicate with the first compartment in parallel circuit arrangement. For example, the compartments may be arranged in a hub-and-spoke arrangement where the first compartment serves as a central hub having direct fluid communication with each of the radially arranged additional compartments (i.e., spokes). However, the same structural connectivity may be formed with different physical arrangements. In additional embodiments, the first compartment and the additional compartments directly communicate in a series circuit arrangement (i.e., arranged in a chain) such that some additional compartments indirectly communicate with the first compartment (i.e., fluid communication occurs through a directly communicating compartment). Combinations of parallel and series connections are also contemplated herein. In some embodiments, at least one of the additional compartments directly communicate with the first compartment while the remaining additional compartments indirectly communicate with the first compartment. Several layers of interconnectivity may be formed in this manner. In some embodiments, the interconnectivity may mimic a biological system. For example, the TS-ECMs and the interconnectivity therebetween may mimic the interconnectivity of parts of an organ, a plurality of organs, and/or an organ system.

The TS-ECM substrates utilized in modeling cancer cell invasion may be selected in a variety of manners. In some embodiments, the TS-ECM substrate of the first compartment is the native TS-ECM of the cancer cells. For example, where breast cancer cells are utilized, the TS-ECM substrate of the first compartment may comprise breast-specific ECM to form a primary cancer colony. As such, cell migration therefrom mimics metastasis of a primary or originating tumor site. In additional embodiments, the TS-ECM substrate of the first compartment may be a foreign TS-ECM. In still additional embodiments, the first compartment may not include a TS-ECM substrate. For example, the first compartment may include a synthetic substrate for culturing the cancer cells. Further, in some embodiments, the cancer cells may be seeded/cultured into more than one compartment (i.e., two or more first compartments). Each of the first compartments may include a different TS-ECM substrate. For example, different types of cancer cells may be cultured in each of the first compartments (e.g., in their native TS-ECM substrates) to model a patient having multiple types of primary cancer. In another example, the same type of cancer cells may be cultured into each of the first compartments to model an advanced cancer that has metastasized to some degree. In additional embodiments, each of the first compartments may include the same TS-ECM substrate to model anatomical pairs of organs. For example, a pair of first compartments may both include lung-specific TS-ECM to model the left and right lungs as part of an organ system.

All of the methods described herein may be performed with a variety of cancer cells types. In many cases, the cancer cells are of a type that is known to commonly metastasize. In some embodiments, the cancer cells are breast cancer cells. In some embodiments, the cancer cells are lung cancer cells. In some embodiments, the cancer cells are prostate cancer cells. In some embodiments, the cancer cells are colon cancer cells. In some embodiments, the cancer cells are rectal cancer cells. Additional types of cancer cells as would be known to one having an ordinary level of skill in the art are also contemplated herein. Further, the methods described herein can be performed with one or more cancer cell subtypes. For example, where the cancer cells are breast cancer cells, the cancer cells may include one or more of luminal A cells, luminal B cells, HER-2 enriched cells, and basal-like cells.

In some embodiments, the selected cancer cells may be foreign to at least one of the utilized TS-ECM substrates. In other words, the cancer cells are utilized with a TS-ECM substrate of a type to which the cells are not native, thus forming a metastatic colony. For example, breast cancer cells may be cultured in one or more of bone-specific ECM, lung-specific ECM, and liver-specific ECM. As such, the resulting colony will be a metastatic colony in that the cancer cells are native to a different niche environment (i.e., breast-specific ECM). However, in some embodiments, the selected cancer cells may be native to the utilized TS-ECM substrate resulting in a culture that models a primary cancer colony, e.g., an originating tumor site. For example, breast cancer cells may be cultured in breast-specific ECM such that the resulting colony is a primary breast cancer colony. In some embodiments, the selected cancer cells may be modeled in both native and foreign TS-ECM substrates. For example, culturing breast cancer cells in breast-specific ECM and one or more of bone-specific ECM, lung-specific ECM, and liver-specific ECM may highlight differences between the primary cancer and the metastatic cancer in a quantifiable manner.

The methods described herein may be performed with cancer cells from a variety of sources. In some embodiments, the cancer cells may be primary tumor cells or tumor-associated cells procured from a human or animal subject. In some embodiments, the cancer cells may be primary tumor cells or tumor-associated cells procured from a prospective patient in order to perform patient-specific therapy evaluation. By culturing cancer cells procured from the patient, various tumor-associated responses in the cell culture may exhibit a greater degree of similarity to the patient's cancer, thus increasing the value of the cell culture as a tool for evaluating the patient's cancer and planning treatment.

In additional embodiments, the cancer cells are procured from a cancer cell line. The cancer cells may be sourced from a variety of cancer cell lines. In some embodiments, the cancer cells are BT-549 breast cancer cells. In some embodiments, the cancer cells are T-47D breast cancer cells. In additional embodiments where breast cancer cells are utilized, the cancer cells may be 600 MPE cells, AMJ13 cells, AU565 cells, BT-20 cells, BT-474 cells, BT-483 cells, Evsa-T cells, Hs 578T cells, MCF7 cells, MDA-MB-231 cells, MDA-MB-468 cells, SkBr3 cells, or ZR-75-1 cells. In some embodiments, the cancer cells are adenocarcinoma A549 lung cancer cells. In some embodiments, the cancer cells are Jacket lung cancer cells. In additional embodiments where lung cancer cells are utilized, the cancer cells may be EKVX cells, HOP-62 cells, HOP-92 cells, NCI-H226 cells, NCI-H23 cells, NCI-H322M cells, NCI-H460 cells, NCI-H522 cells, PC9 cell, L068 cells, LUDLU-1 cells, COR-L105 cells, SKLU1 cells, SKMES1 cells, NCI-H727 cells, LC-2/AD cells, NCIH358 cells, ChaGo-K-1 cells, MOR/CPR cells, MOR/0.4R cells, or MOR/0.2R cells. In some embodiments, the cancer cells are prostate cancer cells, such as DU-145 cells or PC-3 cells. In some embodiments, the cancer cells are colon cancer cells, such as Colo205 cells, HCC-2998 cells, HCT-116 cells, HCT-15 cells, HT29 cells, KM12 cells, or SW-620 cells. Additional types of cancer cells and additional cancer cell lines are additionally contemplated herein, as would be known to a person having an ordinary level of skill in the art. Further, any combination of cancer cell types and/or cancer cell lines could be utilized with the cell culture platform.

In some embodiments, the methods described herein are utilized to evaluate a potential metastatic cancer therapy. For example, the method may comprise applying a potential cancer therapy drug to the one or more TS-ECM substrates and assessing the cell viability. The results may be indicative of the drug's potential as a candidate for metastatic cancer treatment. Further, where multiple different TS-ECM substrates are evaluated, the results may be instructive of the drug's treatment potential specifically with respect to particular metastasis sites.

In some embodiments, the methods described herein are utilized to evaluate a known metastatic cancer therapy. For example, where primary tumor cells from a prospective patient are utilized, one or more known metastatic cancer therapies may be assessed by the described methods in order to attain patient-specific assessment of the one or more known metastatic cancer therapies. In such a case, the methods may be utilized with TS-ECM substrates emulating the niche environments of known or expected sites of metastasis for the patient.

In some embodiments, the method comprises providing a plurality of TS-ECM substrates. As such, the method may comprise assessing the at least one tumor-associated response in each TS-ECM substrate.

The TS-ECM substrates may emulate the niche environment of various tissues. In some embodiments, the TS-ECM substrates may emulate common sites of metastasis. For example, the TS-ECM may be selected from bone-specific ECM, lung-specific ECM, and liver-specific ECM. In additional embodiments, the TS-ECM may be selected from additional niche environments, such as brain-specific ECM, kidney-specific extracellular matrix, skin-specific extracellular matrix, intestine-specific extracellular matrix, heart-specific extracellular matrix, and lymph-specific extracellular matrix. In still additional embodiments, the TS-ECM may emulate a niche environment specific to another tissue. For example, the tissue may be selected from the adrenal gland, amnion, bladder, blood vessel, breast, cartilage, chorion, connective tissue, esophagus, eye, fat, larynx, ligament, microvasculature, muscle, mouth, omentum, ovary, fallopian tube, thyroid, parathyroid, large intestine, small intestine, pancreas, peritoneum, pharynx, placenta membrane, prostate, rectum, smooth muscle, spinal cord, spinal fluid, spleen, stomach, tendon, testes, thymus, umbilical cord, uterus, vagina, or Wharton's Jelly. In some embodiments, the TS-ECM may emulate a region of the anatomy, an organ, or a region of an organ. For example, left and right lungs have unique anatomies and may represent unique TS-ECMs which may be utilized individually or together for direct comparison. In another example, a TS-ECM may represent the large intestine or it may more specifically represent the colon or the rectum.

The TS-ECMs may be derived from a variety of non-metastatic tissue sources. In some embodiments, the tissue source is selected from a human source and an animal source. For example, the tissue may be porcine (i.e., sourced from a pig) or any other animal tissue known to have clinical relevance. In some embodiments, the tissue source is selected from fetal tissue, juvenile tissue, and adult tissue. In some embodiments, the tissue source is selected from healthy tissue, diseased tissue, transgenic tissue, or tissue having a specific disorder or health condition. For example, in some embodiments, the tissue source is fibrotic tissue (i.e., exhibiting tissue fibrosis). The resulting TS-ECM is representative of extracellular matrix from the tissue source, or more generally from tissue having the same relevant characteristics as the tissue source (e.g., juvenile human lung tissue will yield lung-specific ECM representative of a juvenile human's lung tissue).

In some embodiments, the TS-ECM substrates are provided on a cell culture vessel. In some embodiments, the cell culture vessel comprises a tissue culture plate. In some embodiments, the cell culture vessel may be a petri dish or other dish. In some embodiments, the cell culture vessel comprises a flask. Additional types of cell culture vessel as would be known to one having an ordinary level of skill in the art are also contemplated herein. The cell culture vessel may comprise one or more divided regions to be utilized for individual TS-ECM substrates. For example, a tissue culture plate may comprise one or more wells. In some embodiments, the plate comprises 1 well, 3 wells, 6 wells, 12 wells, 24 wells, 48 wells, 96 wells, 384 wells, greater than 384 wells, or any individual value or any range between any two values therein.

In some embodiments, the in vitro cell culture platform utilized herein has a shelf life of about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, or any individual value or any range between any two values therein.

In some embodiments, the in vitro cell culture platform comprises a plurality of TS-ECM substrates. The cell culture platform may be a culture plate having a plurality of divided regions (e.g., wells), where each region includes a TS-ECM substrate. In some embodiments, the plurality of TS-ECM substrates may include a variety of different tissue-specific extracellular matrices in order to emulate multiple niche environments in a single platform. For example, a culture plate may include one or more first wells comprising a first TS-ECM substrate, one or more second wells comprising a second TS-ECM substrate, and one or more third wells comprising a third TS-ECM substrate. In some embodiments, the first TS-ECM substrate comprises bone-specific ECM, the second TS-ECM substrate comprises lung-specific ECM, and the third TS-ECM substrate comprises liver-specific ECM. However, any combination of TS-ECM substrates disclosed herein is contemplated. While a combination of three different TS-ECM substrates is demonstrated, it should be understood that other quantities are contemplated. A culture plate may comprise two, three, four, five, or more different TS-ECM substrates.

In some embodiments, combinations of TS-ECM substrates are selected based on common sites of metastasis for a particular tumor type. In some embodiments, a cell culture platform for modeling breast cancer cells comprises one or more different TS-ECM substrates, each emulating a niche environment selected from bone tissue, liver tissue, lung tissue, and brain tissue. In some embodiments, a cell culture platform for modeling lung cancer cells comprises one or more different TS-ECM substrates each emulating a niche environment selected from bone tissue, liver tissue, opposite lung tissue (e.g., where the cancer cells are from a left lung, the TS-ECM emulates right lung tissue), brain tissue, and adrenal gland tissue. In some embodiments, a cell culture platform for modeling liver cancer cells comprises one or more different TS-ECM substrates, each emulating a niche environment selected from lung tissue, kidney tissue, and lymph tissue (e.g., portal lymph nodes). In some embodiments, a cell culture platform for modeling bone cancer cells comprises a TS-ECM substrate emulating a niche environment specific to lung tissue. In some embodiments, a cell culture platform for modeling brain cancer cells comprises one or more different TS-ECM substrate, each emulating a niche environment selected from spinal cord tissue and spinal fluid.

In additional embodiments, a cell culture platform for modeling bladder cancer cells comprises one or more different TS-ECM substrates, each emulating a niche environment selected from bone tissue, liver tissue, and lung tissue. In some embodiments, a cell culture platform for modeling colon cancer cells and/or rectal cancer cells comprises one or more different TS-ECM substrates, each emulating a niche environment selected from liver tissue, lung tissue, and peritoneal tissue. In some embodiments, a cell culture platform for modeling esophageal cancer cells comprises one or more different TS-ECM substrates, each emulating a niche environment selected from bone tissue, liver tissue, lung tissue, lymph node tissue, and stomach tissue. In some embodiments, a cell culture platform for modeling fallopian tube cancer cells comprises one or more different TS-ECM substrates, each emulating a niche environment selected from bone tissue, liver tissue, lung tissue, kidney tissue, brain tissue, peritoneal tissue, ovarian tissue, and uterine tissue. In some embodiments, a cell culture platform for modeling gallbladder cancer cells comprises one or more different TS-ECM substrates, each emulating a niche environment selected from liver tissue, pancreatic tissue, and lymph node tissue. In some embodiments, a cell culture platform for modeling kidney cancer cells comprises one or more different TS-ECM substrates, each emulating a niche environment selected from bone tissue, liver tissue, lung tissue, brain tissue, adrenal gland tissue, ovarian tissue, and testicular tissue. In some embodiments, a cell culture platform for modeling blood or bone marrow cancer cells (i.e., leukemia) comprises one or more different TS-ECM substrates, each emulating a niche environment selected from liver tissue, spleen tissue, spinal fluid, lymph node tissue, and testicular tissue. In some embodiments, a cell culture platform for modeling mouth cancer cells comprises one or more different TS-ECM substrates, each emulating a niche environment selected from lung tissue and lymph tissue (e.g., neck lymph nodes). In some embodiments, a cell culture platform for modeling oral and/or oropharyngeal cancer cells comprises one or more different TS-ECM substrates, each emulating a niche environment selected from lung tissue, kidney tissue, neck tissue, throat tissue, and prostate tissue. In some embodiments, a cell culture platform for modeling ovarian cancer cells comprises one or more different TS-ECM substrates, each emulating a niche environment selected from liver tissue, lung tissue, spleen tissue, peritoneal tissue, and fallopian tube tissue. In some embodiments, a cell culture platform for modeling pancreatic cancer cells comprises one or more different TS-ECM substrates, each emulating a niche environment selected from liver tissue, lung tissue, and peritoneal tissue. In some embodiments, a cell culture platform for modeling prostate cancer cells comprises one or more different TS-ECM substrates, each emulating a niche environment selected from bone tissue, liver tissue, lung tissue, and adrenal gland tissue. In some embodiments, a cell culture platform for modeling skin cancer cells comprises one or more different TS-ECM substrates, each emulating a niche environment selected from bone tissue, liver tissue, lung tissue, brain tissue, skin tissue, and muscular tissue. In some embodiments, a cell culture platform for modeling stomach cancer cells comprises one or more different TS-ECM substrates, each emulating a niche environment selected from liver tissue, lung tissue, and peritoneal tissue. In some embodiments, a cell culture platform for modeling testicular cancer cells comprises one or more different TS-ECM substrates, each emulating a niche environment selected from liver tissue and lymph node tissue. In some embodiments, a cell culture platform for modeling throat cancer cells comprises one or more different TS-ECM substrates, each emulating a niche environment selected from bone tissue and lung tissue. In some embodiments, a cell culture platform for modeling thyroid cancer cells comprises one or more different TS-ECM substrates, each emulating a niche environment selected from bone tissue, liver tissue, and lung tissue. In some embodiments, a cell culture platform for modeling urethral cancer cells comprises one or more different TS-ECM substrates, each emulating a niche environment selected from bone tissue, liver tissue, lung tissue, kidney tissue, and lymph node tissue. In some embodiments, a cell culture platform for modeling uterine cancer cells comprises one or more different TS-ECM substrates, each emulating a niche environment selected from bone tissue, liver tissue, lung tissue, peritoneal tissue, rectal tissue, bladder tissue, fallopian tube tissue, and vaginal tissue.

In still additional embodiments, a cell culture platform for modeling non-Hodgkin lymphoma cells comprises one or more different TS-ECM substrates, each emulating a niche environment selected from bone tissue, liver tissue, and lung tissue. In some embodiments, a cell culture platform for modeling multiple myeloma cells comprises one or more different TS-ECM substrates, each emulating a niche environment selected from central nervous system tissue (e.g., brain, spinal cord, spinal fluid) and blood. In some embodiments, a cell culture platform for modeling neuroblastoma cells comprises one or more different TS-ECM substrates, each emulating a niche environment selected from liver tissue and adrenal gland tissue. In some embodiments, a cell culture platform for modeling ocular melanoma cells comprises one or more different TS-ECM substrates, each emulating a niche environment selected from bone tissue, liver tissue, and lung tissue. In some embodiments, a cell culture platform for modeling sarcoma cells comprises a TS-ECM substrate emulating a niche environment specific to lung tissue. Additional types of cancer cells and/or additional sets of TS-ECM substrates are contemplated herein as would be known to one having an ordinary level of skill in the art.

In some embodiments, each TS-ECM substrate of the cell culture platform is segregated, i.e., completely physically separated from other TS-ECM substrates. The physical separation must be capable of preventing cell transfer between the TS-ECM substrates, co-mingling of cell culture components, interaction, cross-contamination, or any other influence of one substrate or culture upon another. In some embodiments, the segregation comprises a barrier such as a wall between the TS-ECM substrates. For example, as described, a tissue culture plate with a plurality of wells may be utilized such that the walls of the wells serve as a physical barrier between the TS-ECMs. Other types of barriers may be utilized as would be known to one having an ordinary level of skill in the art. In some embodiments, an adequate amount of physical spacing between TS-ECM substrates may provide sufficient segregation. For example, as described above, a tissue culture plate may include divided regions which are adequately spaced to provide for individual TS-ECM substrates. Further, in some embodiments, multiple plates or vessels may be utilized, where one or more TS-ECMs are provided on each plate or vessel in order to provide segregation. Various additional manners of providing physical separation between substrates as would be known to one having an ordinary level of skill in the art are contemplated herein.

In additional embodiments, each TS-ECM substrate may be compartmentalized, i.e., physically separated from the other TS-ECM substrates to prevent intermixing in a manner that would substantially alter the composition of any of the TS-ECM substrates. Compartmentalized TS-ECM substrates may include a means of fluid communication therebetween. For example, the compartmentalization may allow for some cell transfer, interaction, or other influence of one substrate or culture upon another (e.g., transfer of some molecules or creation of a gradient therebetween). In some embodiments, the TS-ECM substrates may be housed in physically separated compartments as described above (e.g., connected vessels, connected chambers of a vessel, etc.) except with fluid channels extending between the compartments. In some embodiments, the compartments comprise microfluidic chambers on a vessel such as chip (e.g., an organ-on-a-chip system). In some embodiments, each compartment comprises a printed bio-ink in a region of a vessel such as a chip. Further, the fluid communication between compartments may be formed in a variety of manners. In some embodiments, the compartments communicate via interconnecting channels spanning between the compartments. For example, the channels may be microfluidic channels. In some embodiments, the compartments are separated by a porous membrane that allows fluid communication therebetween. The fluid communication may be configured to allow transport of fluids, molecules, cells, or a combination thereof. Additionally, the fluid communication may be arranged in a variety of manners. In some embodiments, each of the additional compartments directly fluidly communicate with the first compartment in parallel circuit arrangement. For example, the compartments may be arranged in a hub-and-spoke arrangement where the first compartment serves as a central hub having direct fluid communication with each of the radially arranged additional compartments (i.e., spokes). However, the same structural connectivity may be formed with different physical arrangements. In additional embodiments, the first compartment and the additional compartments directly communicate in a series circuit arrangement (i.e., arranged in a chain) such that some additional compartments indirectly communicate with the first compartment (i.e., fluid communication occurs through a directly communicating compartment). Combinations of parallel and series connections are also contemplated herein. In some embodiments, at least one of the additional compartments directly communicate with the first compartment while the remaining additional compartments indirectly communicate with the first compartment. Several layers of interconnectivity may be formed in this manner. In some embodiments, the interconnectivity may mimic a biological system. For example, the TS-ECMs and the interconnectivity therebetween may mimic the interconnectivity of parts of an organ, a plurality of organs, and/or an organ system.

The TS-ECM may be processed and provided in a variety of substrate formats. In some embodiments, the format of the TS-ECM substrate may be selected from a hydrogel, a scaffold (e.g., an acellular scaffold), a surface coating, a sponge, fibers (e.g., electrospun fibers), liquid solution, media supplement, and bio-ink (e.g., printable bio-ink).

The TS-ECM has a specified composition that emulates the ECM found in a specific native tissue. As such, the composition of each TS-ECM may vary. Each TS-ECM may comprise a different combination of proteoglycans, collagens, elastins, multiadhesive proteins, hyaluronic acid, CAMs, and additional components. Each of these components may have subtypes, the presence of each of which may vary from one TS-ECM to another TS-ECM. Each TS-ECM may be characterized by the presence or absence of one or more components. Further, the concentration of each component may vary from one TS-ECM to another TS-ECM. These variations result in each TS-ECM having unique physical characteristics, such as architecture and stiffness, and unique cell interaction characteristics, such as gene expression, ECM remodeling, and cell proliferation.

In some embodiments, bone-specific ECM may comprise about 580-620 μg/mL collagens, about 40-50 μg/mL elastins, and about 10-20 μg/mL glycosaminoglycans. In some embodiments, the bone-specific ECM has an elastic modulus of about 6.6 kPa. However, the elastic modulus may be about 6 to about 25 kPa, about 6 to about 10⁵ kPa, or individual values or ranges therebetween. In some embodiments, the elastic modulus may be similar to the elastic modulus of natural bone tissue. In some embodiments, the collagen comprises type I α1, type I α2, type II α1, type III α1, type V α2, type VI α2, type VI α3, type VIII α1, type IX α2, type X α1, type XI α1, type XI α2, type XII α2, type XIV α1, and/or procollagen α1(V) collagen chains. In some embodiments, the bone-specific ECM comprises proteoglycans including aggrecan core protein, asporin, decorin, fibromodullin, heparan sulfate proteoglycan 2, lumican, osteoglycin/mimecan, osteomodulin, and/or proline/arginine-rich end leucine-rich repeat protein. In some embodiments, the bone-specific ECM comprises glycoproteins including AE binding protein 1, alpha-2-HS-glycoprotein, bone gamma-carboxyglutamate protein, biglycan, ECM protein 2, elastin, fibrillin 1, fibrinogen beta chain, fibrinogen gamma chain, fibronectin 1, periostin, osteonectin, transforming growth factor-beta-induced protein, thrombospondin 1, tenascin C, tenascin N, and/or vitronectin. In some embodiments, the bone-specific ECM comprises matrix-associated factors including albumin, annexin A2, acidic chitinase, creatine kinase B, mucin 5AC (oligomeric mucus/gel-forming), and/or collectin subfamily member 12 (collectin-12). In some embodiments, the bone-specific ECM comprises other structural factors including actin γ2 and/or vimentin. In some embodiments, the bone-specific ECM comprises ECM regulators including prothrombin, coagulation factor IX, coagulation factor X, inter-alpha (globulin) inhibitor H4, and/or serpin peptidase inhibitor, clade F. In some embodiments, the bone-specific ECM comprises matrisome-secreted factors including olfactomedin. In some embodiments, the bone-specific ECM comprises immune factors including complement component 3 (C3) and/or immunoglobulin G heavy chain. In some embodiments, the bone-specific ECM comprises marrow-associated factors including hemoglobin subunit α and/or hemoglobin subunit β.

In some embodiments, lung-specific ECM may comprise about 400-530 μg/mL collagens, about 40-50 μg/mL elastins, and about 3-5 μg/mL glycosaminoglycans. In some embodiments, the lung-specific ECM has an elastic modulus of about 3.1 kPa. However, the elastic modulus may be about 3 to about 6 kPa, about 2 to about 8 kPa, about 2 to about 12 kPa, or individual values or ranges therebetween. In some embodiments, the elastic modulus may be similar to the elastic modulus of natural lung tissue. In some embodiments, the collagen comprises type I α1, type I α2, type II α1, type III α1, type IV α1, type IV α2, type IV α3, type IV α4, type IV α5, type V α2, type VI α2, type VI α3, type VI α5, type VIII α1, type IX α2, type XI α1, type XI α2, type XVI α1, and/or procollagen α1(V) collagen chains. In some embodiments, the lung-specific ECM comprises proteoglycans including hyaluronan, heparan sulfate, aggrecan core protein, hyaluronan and proteoglycan link protein 1, and/or heparan sulfate proteoglycan 2. In some embodiments, the lung-specific ECM comprises glycoproteins including dermatopontin, elastin, fibrillin 1, fibulin 5, laminin γ1, laminin subunit α (e.g., α5), laminin subunit β (e.g., β2), microfibril associated protein 4, nidogen 1, and/or periostin. In some embodiments, the lung-specific ECM comprises matrix-associated factors including albumin and/or acidic chitinase. In some embodiments, the lung-specific ECM comprises other structural factors including actin γ2 and/or aquaporin-1. In some embodiments, the lung-specific ECM comprises matrisome-secreted factors including hornerin.

In some embodiments, liver-specific ECM may comprise about 1100-1300 μg/mL collagens, about 120-150 μg/mL elastins, and about 5-15 μg/mL glycosaminoglycans. In some embodiments, the liver-specific ECM has an elastic modulus of about 2.8 kPa. However, the elastic modulus may be about 2 to about 7 kPa, about 2 to about 10 kPa, about 2 to about 15 kPa, about 7 to about 15 kPa, or individual values or ranges therebetween. In some embodiments, the elastic modulus may be similar to the elastic modulus of natural liver tissue. In some embodiments, the collagen comprises type I α1, type I α2, type II α1, type III α1, type IV α2, type V α2, type VI 3, and type VI α5 collagen chains. In some embodiments, the liver-specific ECM comprises proteoglycans including heparan sulfate and/or heparan sulfate proteoglycan 2. In some embodiments, the liver-specific ECM comprises glycoproteins including EGF-contained fibulin-like ECM protein, elastin, fibrillin 1, fibrillin 2, laminin γ1, saposin-B-val, prostate stem cell antigen, and/or von Willebrand factor. In some embodiments, the liver-specific ECM comprises matrix-associated factors including albumin, acidic chitinase, mucin 5AC (oligomeric mucus/gel-forming), collectin-12, mucin 6 (oligomeric mucus/gel-forming), and/or trefoil factor 2. In some embodiments, the liver-specific ECM comprises other structural factors including actin, keratin type II cytoskeletal 1, keratin type I cytoskeletal 10, keratin type II cytoskeletal 2 epidermal, keratin type I cytoskeletal 9, myosin heavy chain 9, and/or tubulin beta chain. In some embodiments, the liver-specific ECM comprises ECM regulators including granulin precursor.

The composition of bone-specific ECM, lung-specific ECM, and liver-specific ECM are summarized in Table 1. However, these compositions are exemplary in nature and the TS-ECM profiles may vary therefrom as to any number of components.

In some embodiments, the substrates may further include additional components beyond the TS-ECM components. In some embodiments, the substrates may include cell culture media, media supplements, or components thereof. In some embodiments, the substrates may include one or more of amino acids, glucose, salts, vitamins, carbohydrates, proteins, peptides, trace elements, other nutrients, extracts, additives, gases, or organic compounds. Additional components for the proper growth, maintenance and/or modeling of cells as would be known to one having an ordinary level of skill in the art are also contemplated herein.

The method of using a cell culture platform may further be adapted in any manner described herein with respect to the cell culture platform, the method of making the cell culture platform, and the kit for forming a cell culture platform.

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples:

### EXAMPLES

### Example 1—Evaluation of Reconstituted TS-ECM Substrate

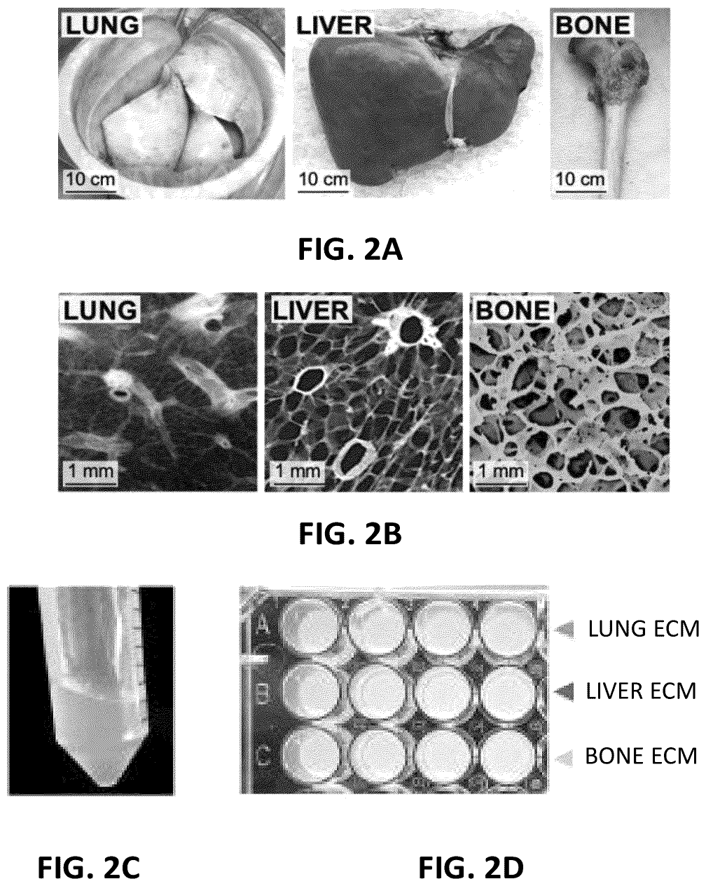

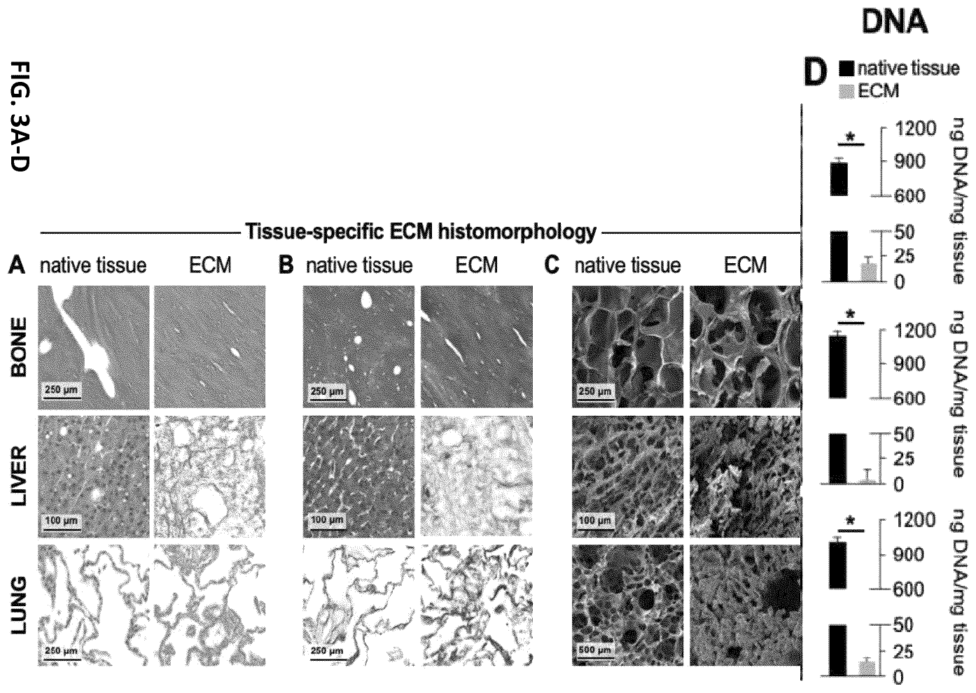

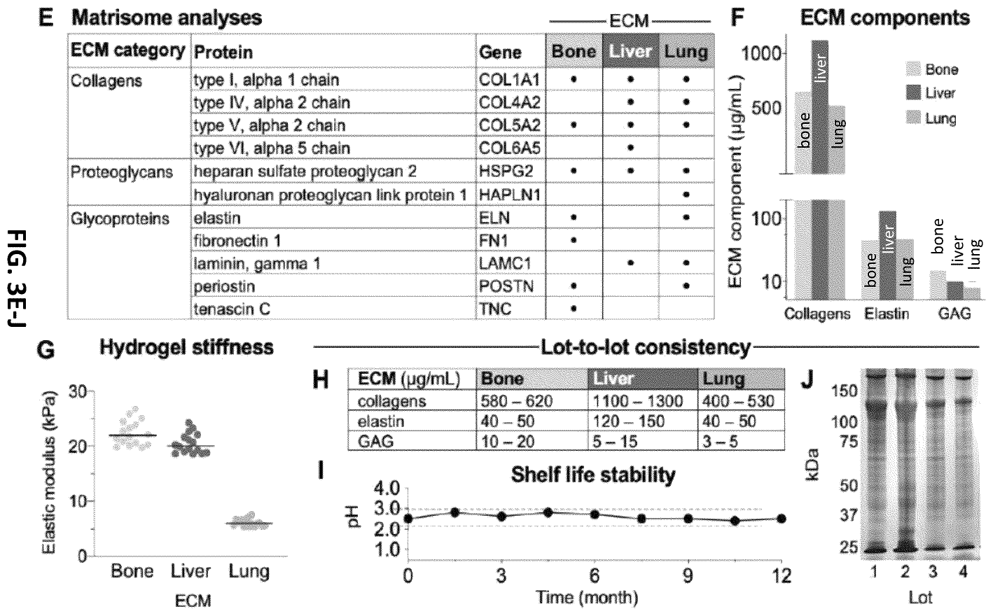

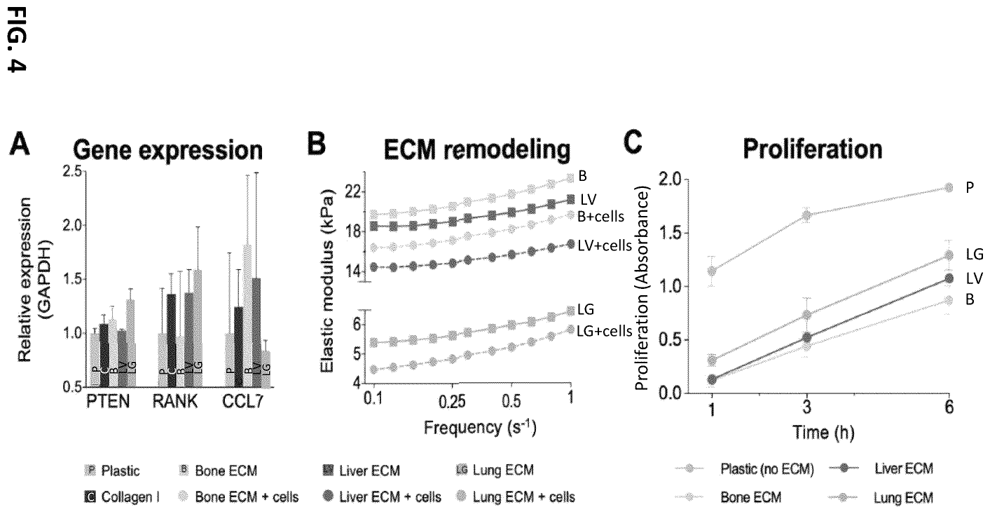

Female human bones (femurs), livers, and lungs were procured (as shown in FIG. 2A) through approved Institutional Review Board protocols. The tissue samples were sectioned and washed with combinations of chemical, detergent, and enzymatic reagents to obtain acellular human bone-specific ECM, liver-specific ECM, and lung-specific ECM (as shown in FIG. 2B). The acellular samples were solubilized (as shown in FIG. 2C) and reconstituted by a reagent to yield bone-specific ECM, liver-specific ECM, and lung-specific ECM in the form of hydrogels plated on a 12-well plate (as shown in FIG. 2D). The histomorphology of the TS-ECMs was evaluated through hematoxylin and eosin staining (results shown in FIG. 3A), trichrome staining (FIG. 3B), and scanning electron micrographs (FIG. 3C). Evaluation revealed high similarity of the TS-ECMs to native tissues, with no discernible nuclei (FIGS. 3A-3B). Further, comparison of the ECM architecture between the different tissues revealed drastic differences (FIG. 3C), indicative of the specificity of the ECM to each tissue. Comparison of the nuclear material present in the native tissue and the isolated TS-ECMs revealed that greater than 99% of nuclear material was removed in the isolated TS-ECMs (FIG. 3D; * p<0.001). Further, biochemical analyses were performed, including proteomics (FIG. 3E) and quantification of the ECM components (FIG. 3F), in order to appraise the tissue-specific profiles of each ECM. Compositional consistency across lots of the TS-ECMs was evaluated to establish compositional ranges of ECM components (FIG. 3H). For example, FIG. 3J illustrates representative gel electrophoresis results between four lots of liver-specific ECM hydrogel, demonstrating the level of compositional consistency. Stiffness tests were performed on the hydrogels, revealing that each TS-ECM has a distinct mechanical stiffness (FIG. 3G). Shelf life was also evaluated to establish 1-year shelf life stability. For example, FIG. 3I illustrates representative pH stability assessment of lung-specific ECM hydrogel, demonstrating at least 1-year shelf life stability.

### Example 2—Evaluation of Breast Cancer Cells in TS-ECM Substrates

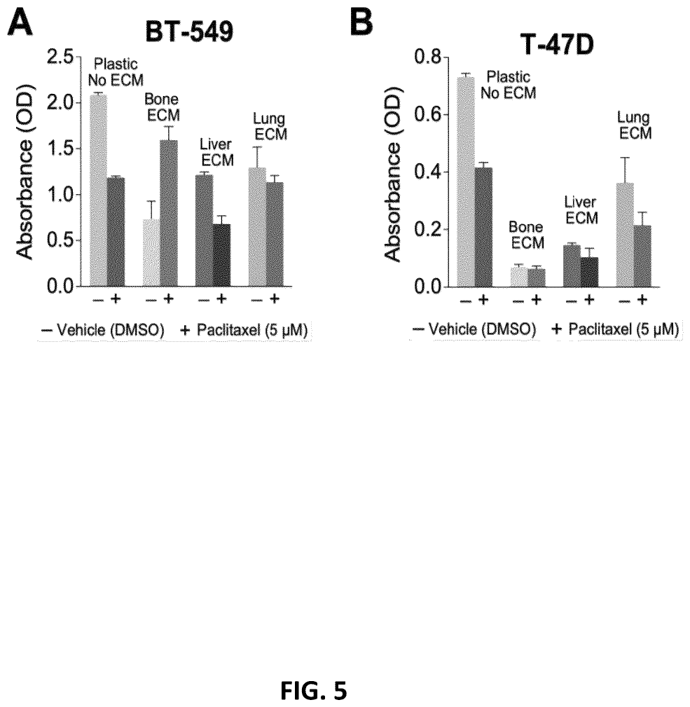

Human metastatic breast cancer cells (BT-549: ER⁻PR⁻HER2⁻; MCF-7, T-47D: ER⁺PR⁺HER2⁻) were cultured in human bone-specific ECM, liver-specific ECM, and lung-specific ECM hydrogels for 24 hours. Drug testing was performed using (1) 4-Hydroxytamoxifen (4-OHT), which is used to treat ER breast cancer; and (2) Fulvestrant and Palbociclib, which is used to treat metastatic ER⁺PR⁺HER2⁻ breast cancer. Colony formation was assessed after 19 days and cell viability was analyzed after 2 days of treatment with Paclitaxel, a chemotherapy medication. Gene expression profiles and regulation of tumor-associated genes were evaluated by analyzing RNA expression of specific tumor-associated genes by the BT-549 cells (results shown in FIG. 4A), illustrating differences in gene expression compared to cells in other substrates. ECM interactions and remodeling was evaluated by analyzing changes in mechanical stiffness of the TS-ECM substrates in response to the BT-549 cells (FIG. 4B). Proliferation of BT-549 cells was evaluated in each TS-ECM substrate as compared to proliferation on a plastic substrate (FIG. 4C). The results indicate that TS-ECM hydrogels support metastatic human breast cancer cell behavior that is specific to each metastatic niche environment. Differential response to drug treatment and viability response to two days of Paclitaxel treatment was evaluated for BT-549 cells and T-47D cells in each TS-ECM substrate by measuring optical density (OD). Results are shown in FIGS. 5A-5B, illustrating that metastatic human breast cancer cells demonstrate tissue-specific responses in each metastatic niche environment.

### Example 3—Evaluation of Lung Cancer Cells in Lung-Specific ECM Substrate

Cell Expansion. Human lung adenocarcinoma A549 cells (CCL-185, ATCC) were cultured using F12K Medium (30-2004, ATCC) and 10% fetal bovine serum. Human lung adenocarcinoma Jacket cells (CB030-000001, Cellaria) were cultured in Basal Renaissance Essential Tumor Medium (CM-0001, Cellaria) with RETM supplement, 25 ng/mL cholera toxin, and 5% heat-inactivated fetal bovine serum (SH3007103HI, HyClone). Both cell types were cultured under standard conditions (37° C., 100% humidity, 5% C02), and passaged at 80% confluency.

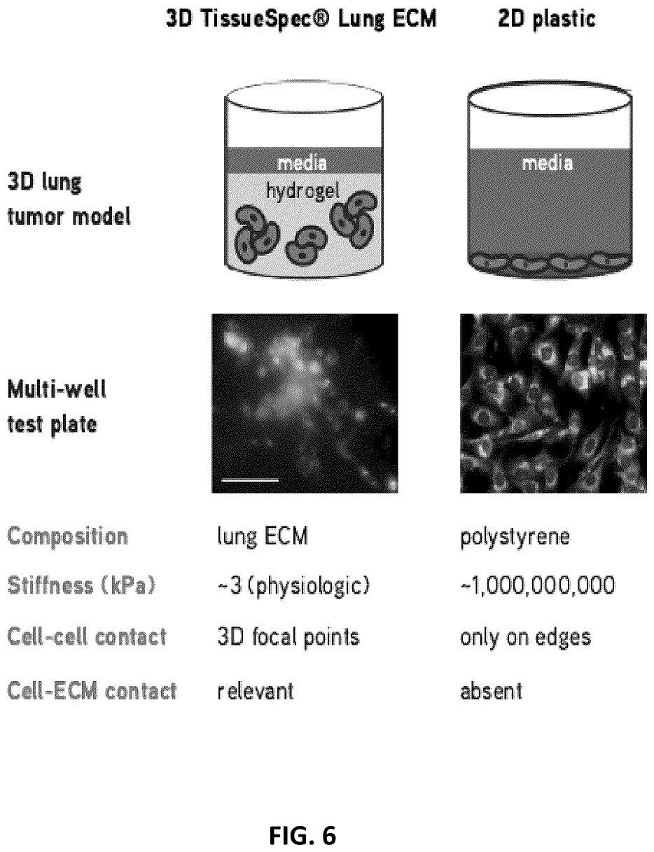

Forming 3D Lung Tumor Model. The 3D lung tumor models were prepared by mixing 5,000 lung adenocarcinoma cells with 40 μL TissueSpec® Lung ECM Hydrogel (MTSLG101, Xylyx Bio) at 4 mg/mL, or Matrigel® (354234, Corning) at 4 mg/mL in 96-well plates. Cells were incubated at 37° C. for 1 hour to allow substrates to gel. After incubation, 100 μL culture medium was added to each well. Cells were also cultured directly on tissue culture plastic as a control without ECM. Tissue culture plastic without ECM and Matrigel® (a basement membrane extract from the Engelbreth-Holm-Swarm murine sarcoma) served as comparative substrates. Models are depicted in FIG. 6 (scale bar: 100 μm).

Drug Treatment. KPT-185 (S7125, Selleckchem), Erlotinib (S7786, Selleckchem), and Etoposide (S1225, Selleckchem) were dissolved in DMSO to obtain 1 mM stock solutions. Cells were cultured for 24 hours, then treated with 100 μL media containing drug for 72 hours. Drug doses were 0.1, 0.5, 1, 5, and 10 μM. In parallel, as negative controls, A549 and Jacket cells were treated with media containing only DMSO at the same concentration of each drug preparation. After 72 hours, cell viability was assayed. All assays were performed with five technical replicates.

Cell Proliferation Assay. Cell proliferation was assessed using a Cell Proliferation Kit II (XTT, 11465015001, Sigma-Aldrich) according to the manufacturer's instructions. The assay reagent XTT is a second-generation tetrazolium dye that is reduced to a soluble orange-colored formazan derivative detectable in real-time. An Assay Protocol was established for use with TissueSpec® Lung ECM Hydrogels. Briefly, 50 μL XTT reagent was added to each well containing 100 μL culture medium. After 4 hours, absorbance was measured using a spectrophotometer at 492 nm and 690 nm as reference.

Data Analysis. Absorbance values lower than control (DMSO) indicated reduction of cell viability. Half maximal inhibitory concentration (IC50) values were calculated for each drug using statistical analysis software (Prism, GraphPad) and nonlinear regression analysis.

Results. To demonstrate the utility of the 3D lung tumor model with lung ECM, the effects of three anti-cancer drugs (two currently used to treat non-small cell lung cancer) were investigated. KPT-185, Etoposide, and Erlotinib were prepared at a range of concentrations, and the viability of lung adenocarcinoma cells cultured in 3D TissueSpec® Lung ECM Hydrogel was evaluated and compared to cells cultured on 2D plastic without ECM.

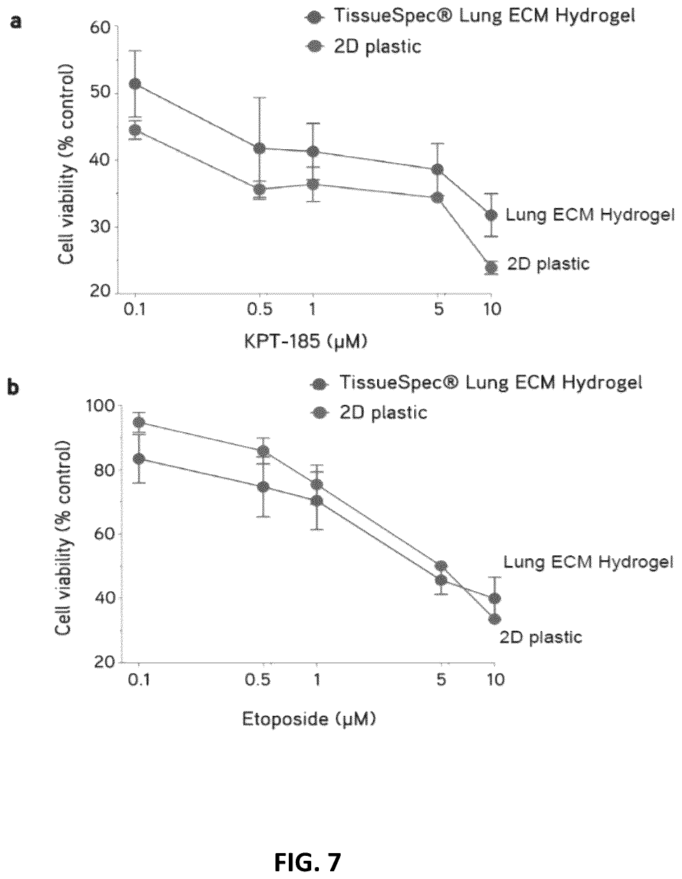

The effects of KPT-185 (FIG. 7A) and Etoposide (FIG. 7B) on Jacket lung adenocarcinoma cells cultured in 3D TissueSpec® Lung ECM Hydrogel and on 2D plastic were quantified. Cell viability in response to a range of KPT-185 doses (0.1-10 μM) is shown in FIG. 7A (data represent mean±standard error mean of five technical repeats).

KPT-185 induced dose-dependent growth inhibition of Jacket cells, which were more resistant in 3D TissueSpec® Lung ECM Hydrogel than on plastic without ECM for all doses evaluated. Etoposide showed dose-dependent growth inhibition in both substrates, however, contrary to the effects of KPT-185, Jacket cells were moderately more sensitive in 3D TissueSpec® Lung ECM Hydrogel than on 2D plastic in the range of 0.1-5 μM (see FIG. 7B). Notably, however, at 10 μM, the highest dose of Etoposide in this study, Jacket cells were more resistant in 3D TissueSpec® Lung ECM Hydrogel than on 2D plastic without ECM.

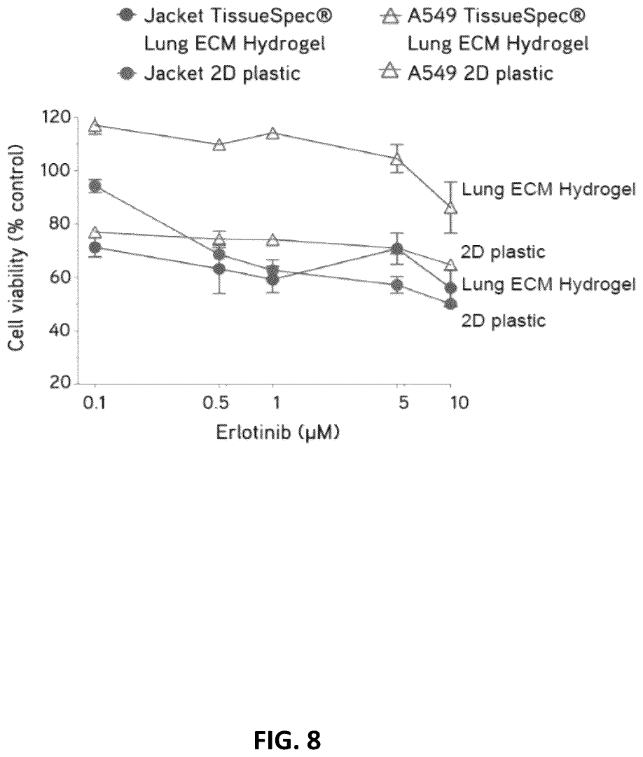

Treatment of A549 cells with a range of doses of Erlotinib (0.1-10 μM) showed dose-dependent growth inhibition, with A549 cells more resistant in 3D TissueSpec® Lung ECM Hydrogel than on plastic. Similarly, Jacket cells showed a more resistant profile in 3D TissueSpec® Lung ECM Hydrogel than on 2D plastic, but only at concentrations above 5 μM. Results are shown in FIG. 8 (data represent mean±standard error mean of five technical repeats).

Based on these studies, half maximal inhibitory concentration (IC50) values of Erlotinib, KPT-185, and Etoposide, are reported in Table 3. The IC50 values of Erlotinib show that A549 cells were more resistant than Jackets cells in all substrates. Interestingly, A549 cells in 3D TissueSpec® Lung ECM Hydrogel had an IC50 value of 134.2 μM—more than 1800% higher than the IC50 value of Jacket cells in the same substrate (7.5 μM). Conversely, IC50 values of Etoposide show that Jacket cells in 3D TissueSpec® Lung ECM Hydrogel were slightly more sensitive (3.7 μM) than Jacket cells on 2D plastic (4.4 μM). A similar trend was observed with IC50 values of KPT-185, where Jacket cells were more resistant in 3D TissueSpec® Lung ECM Hydrogel (0.4 μM) than on plastic (0.2 μM).

TABLE 3
IC50 values for A549 and Jacket cells. Data represents
mean ± standard error mean of five technical replicates.

IC₅₀(μM)

| Substrate | A549 | Jacket |
|---|---|---|
| Erlotinib | | |
| TissueSpec ® Lung ECM | 134.2 | 7.5 |
| Plastic (no ECM) | 36.4 | 5.2 |
| Matrigel ® | 57.5 | 8.7 |
| KPT-185 | | |
| TissueSpec ® Lung ECM | 2.3 | 0.4 |
| Plastic (no ECM) | 8.8 | 0.2 |
| Matrigel ® | 4.2 | 4.3 |
| Etoposide | | |
| TissueSpec ® Lung ECM | 16.6 | 3.7 |
| Plastic (no ECM) | 40.3 | 4.4 |
| Matrigel ® | 35.5 | 15.8 |

A common 3D lung tumor model involves culturing A549 cells in Matrigel®. Comparison of IC50 values of A549 and Jacket adenocarcinoma cells cultured in Matrigel® revealed that Jacket cells were more sensitive than A549 cells to Etoposide and KPT-185 (Table 3). Notably, IC50 values of cells in Matrigel® were considerably higher than IC50 values of cells in TissueSpec® Lung ECM Hydrogel, consistent with previous studies where MCF-7 and MB-231 breast cancer cells were shown to be more resistant to doxorubicin when cultured in Matrigel®.

Notably, Jacket cells in TissueSpec® Lung ECM Hydrogel yielded highly consistent IC50 values. In two separate rounds of testing, Etoposide yielded IC50 values of 3.8 μM and 4.0 μM, and KPT-185 yielded the exact same IC50 value of 0.4 μM in both studies. By contrast, Jacket cells in Matrigel® yielded inconsistent, highly variable IC50 values across the same series of testing. IC50 values for Etoposide were 0.7 μM and 18.3 μM. Moreover, IC50 values for KPT-185 were 0.2 μM and 5.3 μM, suggesting that Matrigel® is highly inconsistent across tests and lots, and is therefore a poor choice of substrate for reproducible drug testing.

Overall, these data indicate that A549 cells are more resistant than Jacket cells to KPT-185, Etoposide, and Erlotinib in the 3D lung tumor model with TissueSpec® Lung ECM Hydrogel, and the other substrates evaluated. Furthermore, Jacket cells are more resistant to Etoposide and Erlotinib when cultured in a 3D environment compared to a 2D surface.

The difference in IC50 values of the three drugs in this study are attributable to the different cellular mechanism that each drug targets as well as expression of and access to each target. Erlotinib is an inhibitor of epidermal growth factor receptor (EGFR) tyrosine kinase, whereas Etoposide inhibits DNA topoisomerase II, which prevents DNA ligation and ultimately causes DNA strands to break. KPT-185 induces apoptosis as a selective inhibitor of nuclear export (SINE) compound. Additionally, variable rates of proliferation of different cell types and densities can significantly affect drug response. Another important consideration for drug testing in 3D culture systems is drug permeability through the ECM and cell membrane. Diffusion assays demonstrated that CellTracker Red CMTPX (686.2 g/mol) has a diffusion speed through TissueSpec® Lung ECM Hydrogel ≥1.3 mm/h.

Jacket cells were derived directly from a patient lung adenocarcinoma, and are therefore patient-specific, lack accumulation of genetic mutations through serial expansion and passaging, and likely represent a more predictive lung cancer model than A549 cells, a hypotriploid cell line. A 3D lung tumor model comprised of patient-specific lung tumor cells in TissueSpec® Lung ECM Hydrogel resembles the in-vivo human disease environment significantly more closely than conventional 2D or 3D models, and enables drug developers to obtain more physiologic cellular responses during drug testing.

### Example 4—Migration of Lung Cancer Cells in TS-ECM Substrates

Methods. TissueSpec® Bone, Liver, or Lung ECM Hydrogels, collage I gel, or plastic (no ECM) were added to the bottom of wells as chemoattractants. Lung adenocarcinoma cells (Jacket, Cellaria) were then cultured on transwell inserts with 8 m pores. After 24 hours, migration was assessed.

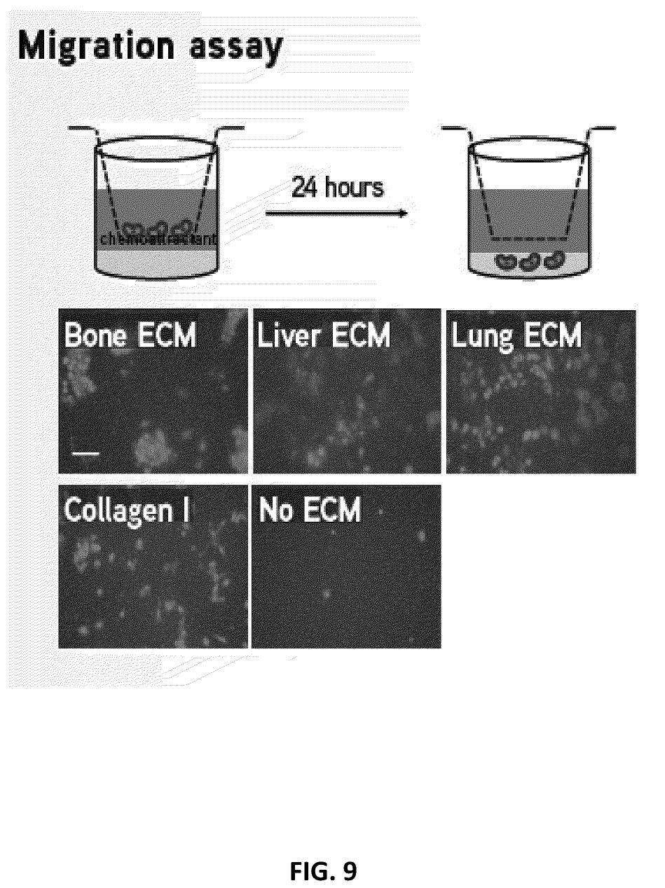

Results. Adenocarcinoma cells showed greater migration toward ECM substrates, and organized differently in each tissue-specific ECM. Notably, clusters formed in Bone ECM. Results shown in FIG. 9 (scale bar: 100 m).

### Example 5—Invasion of Lung Cancer Cells in Lung-Specific ECM Substrate

Methods. Cells were cultured on transwell inserts coated with TissueSpec® Lung ECM Hydrogel or Matrigel. Media with 10% serum was added to the lower compartment, and media without serum (or +serum as control) was added to the upper compartment. After 24 hours, cells were scratched/removed from hydrogel surfaces, inserts were stained with crystal violet, and 5 random 10× fields were quantified.

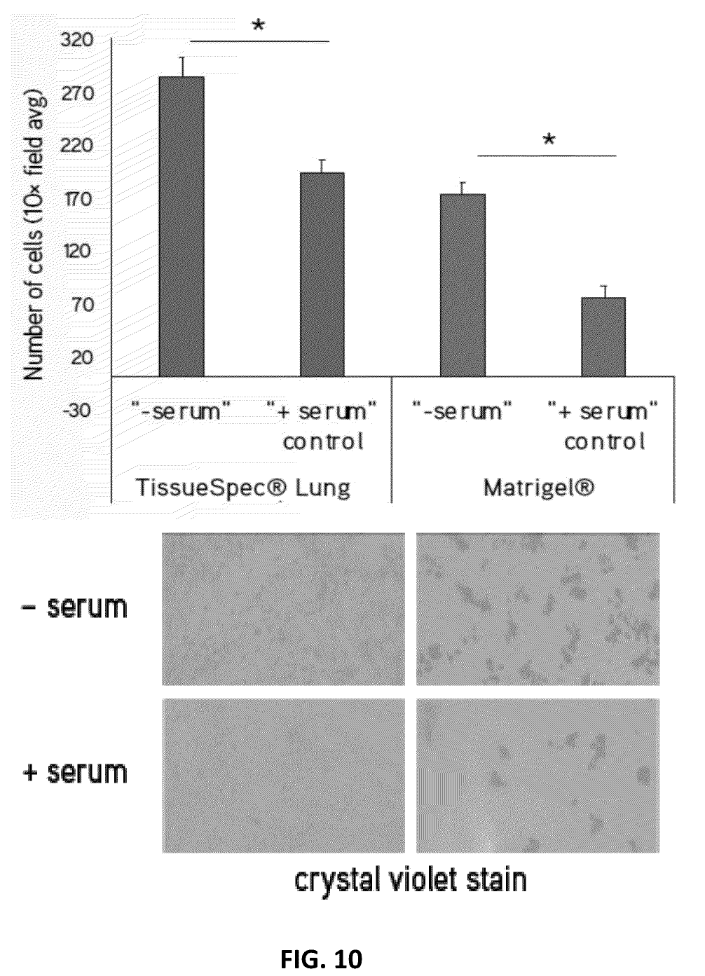

Results. A549 cells cultured on TissueSpec® Lung ECM Hydrogel exhibit significantly greater motility and invasiveness than cells cultured on Matrigel (p<0.05). Results are shown in FIG. 10.

### Example 6—Gene Expression of Metastatic Breast Cancer Cells on TS-ECM Substrates

Methods. Metastatic breast cancer cells (BT-549) were cultured on NativeCoat ECM, collagen, or plastic for 24 hours. Gene expression was normalized to BT-549 cells cultured on plastic.

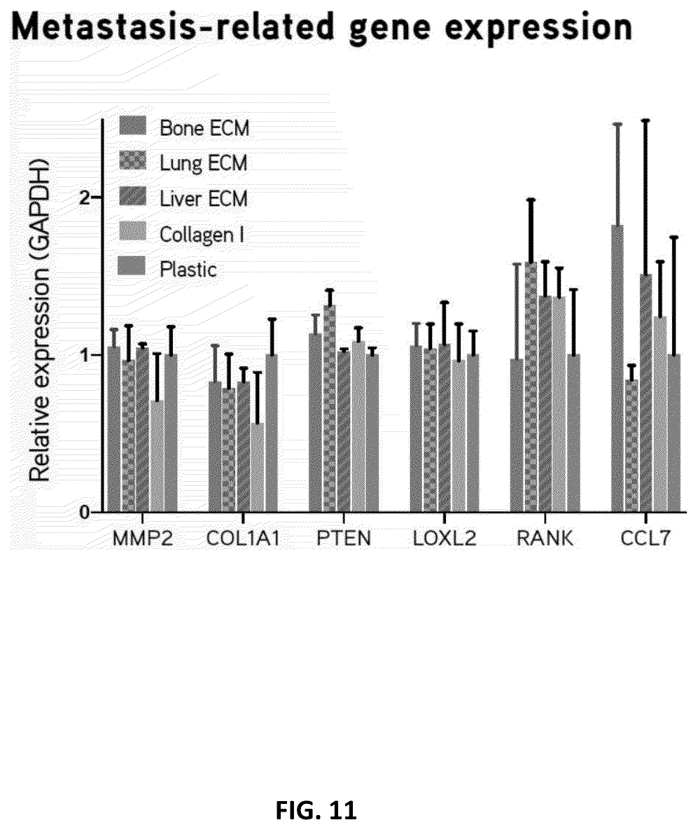

Results. Cells cultured on NativeCoat Bone, Liver, and Lung ECM expressed higher levels of RANK mRNA. Cells cultured on NativeCoat Bone ECM expressed higher levels of CCL7 mRNA. Results are shown in FIG. 11.

### Example 7—ECM Remodeling in TS-ECM Substrates

Methods. Rheometric testing was conducted on TissueSpec® Bone, Liver, and Lung ECM Hydrogels with and without 5×10⁵ Jacket lung adenocarcinoma cells after 48 hours.

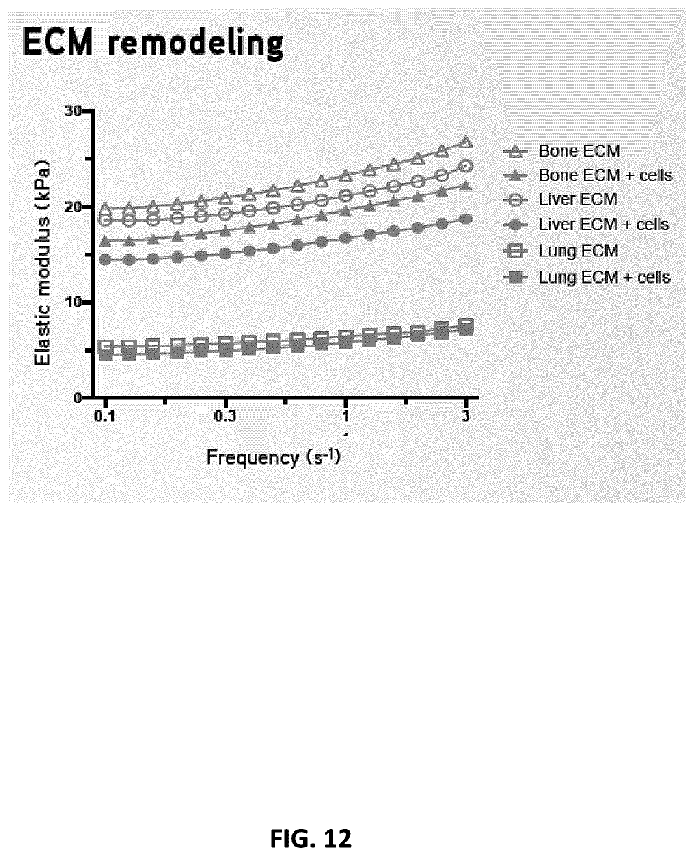

Results. Bone and Liver ECM Hydrogels had reduced elastic moduli, whereas modulus of Lung ECM Hydrogel did not change. Results are shown in FIG. 12.

### Example 8—Drug Response of Lung Cancer Cells in Lung-Specific ECM Substrate

Methods. 5000 cells were cultured in TissueSpec® Lung Hydrogel, Matrigel, or tissue culture plastic (no ECM). After 24 hours, drugs reconstituted in DMSO were added. Cells that received DMSO only were cultured as controls. Cells were cultured for 72 hours, then MTT reagent was added and incubated for 4 hours. Cell number was normalized to average cell number of DMSO control for various doses. IC50 calculations were completed (non-linear fit, GraphPad).

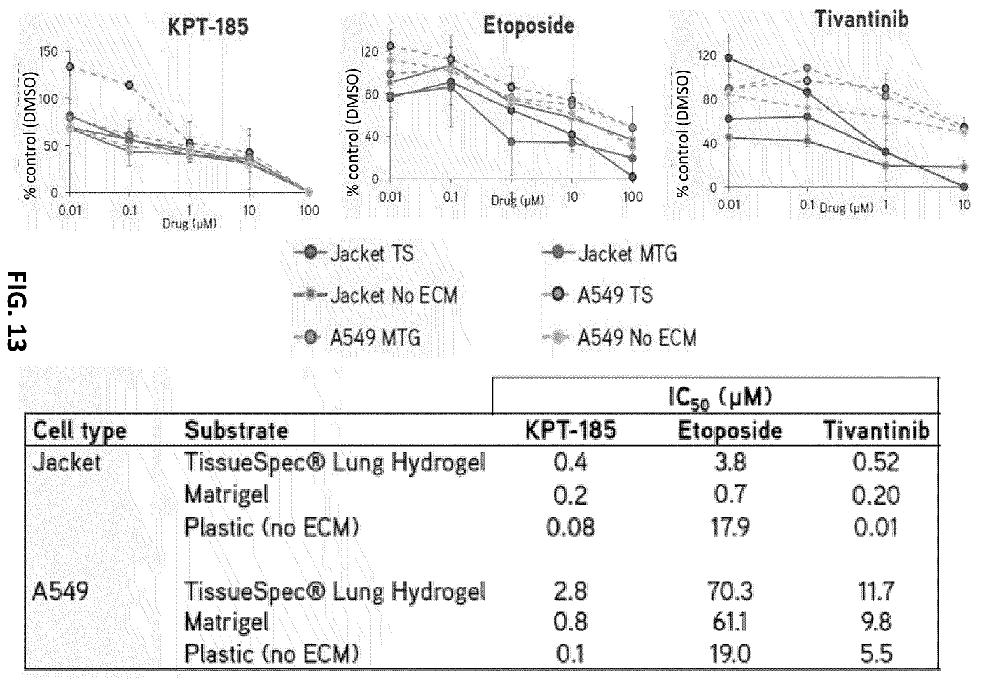

Results. Cells cultured in TissueSpec® Lung Hydrogel exhibited distinct drug resistance profiles, which may indicate a more predictive physiological response. Jacket cells exhibited lower IC50 values compared to A549 cells. Results are shown in FIG. 13.

### Example 9—Migration of Jacket and A549 Cells in Lung-Specific ECM Substrate

Methods. Cells were cultured on a surface coated with NativeCoat Lung ECM, Matrigel, or uncoated surface. Initially, a scratch wound was made using a 200 μL micropipette tip. After 24 hours, cells migrated into scratched areas.

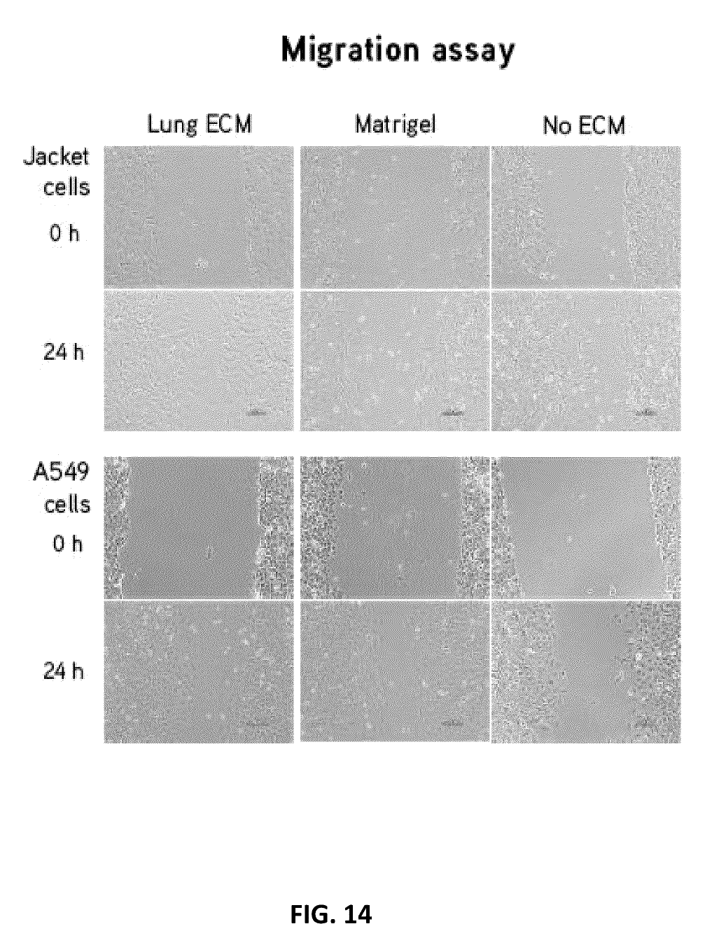

Results. Jacket and A549 cells display migration when cultured on surfaces coated with NativeCoat Lung ECM. Results are shown in FIG. 14.

### Example 10—Cancer-Related Gene Expression of Cancer Cells in Lung-Specific ECM Substrate

Methods. Cells were cultured in 3D TissueSpec® Lung ECM Hydrogel, Matrigel, or on 2D tissue culture plastic (no ECM) for 7 days. Gene expression was normalized to GAPDH using the 2^{\hat{ }ΔΔCt} relative to A549 cells cultured in Matrigel.

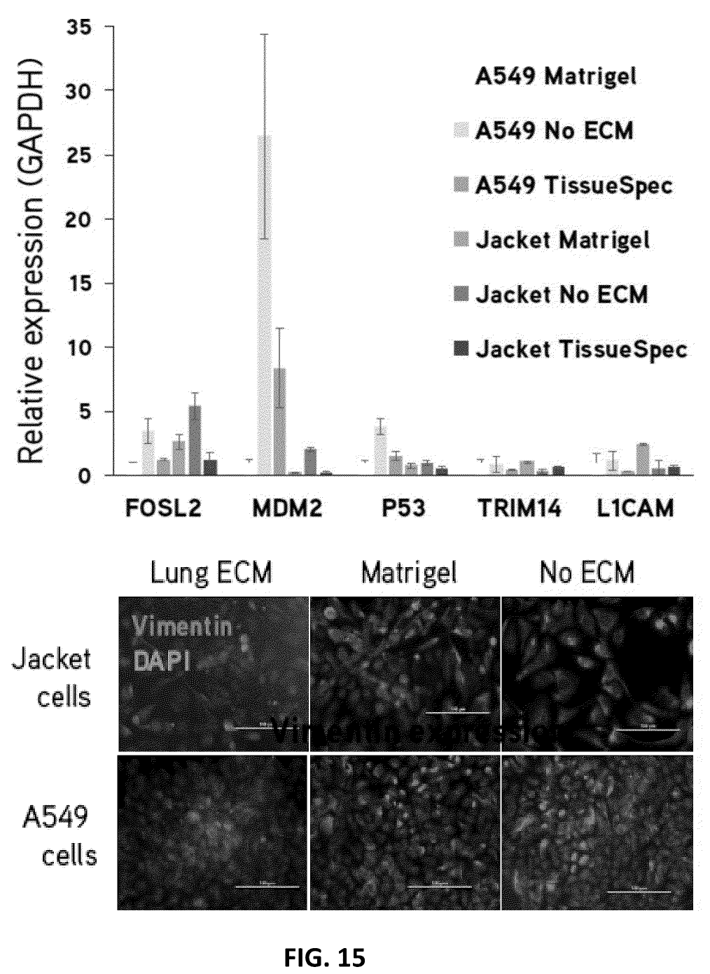

Results. Cells that were cultured in TissueSpec® Lung ECM Hydrogel exhibited lower cancer-related gene expression than cells cultured in Matrigel or on tissue culture plastic with no ECM. A549 and Jacket cells cultured in TissueSpec® Lung ECM Hydrogel exhibited lower Vimentin expression level and abundance than cells cultured in Matrigel or on tissue culture plastic with no ECM. Results are shown in FIG. 15.

### Example 11—Viability of Breast Cancer Cells Embedded in TissueSpec Bone, Liver, and Lung ECM Hydrogels and Treated with Tamoxifen

Methods. Viability assays were performed on breast cancer cells T47-D embedded in TissueSpec® Bone, Liver, and Lung ECM Hydrogels and treated with Tamoxifen (20 M and 40 M) or vehicle (DMSO) for 72 hours. Matrigel and plastic were used as control substrates.

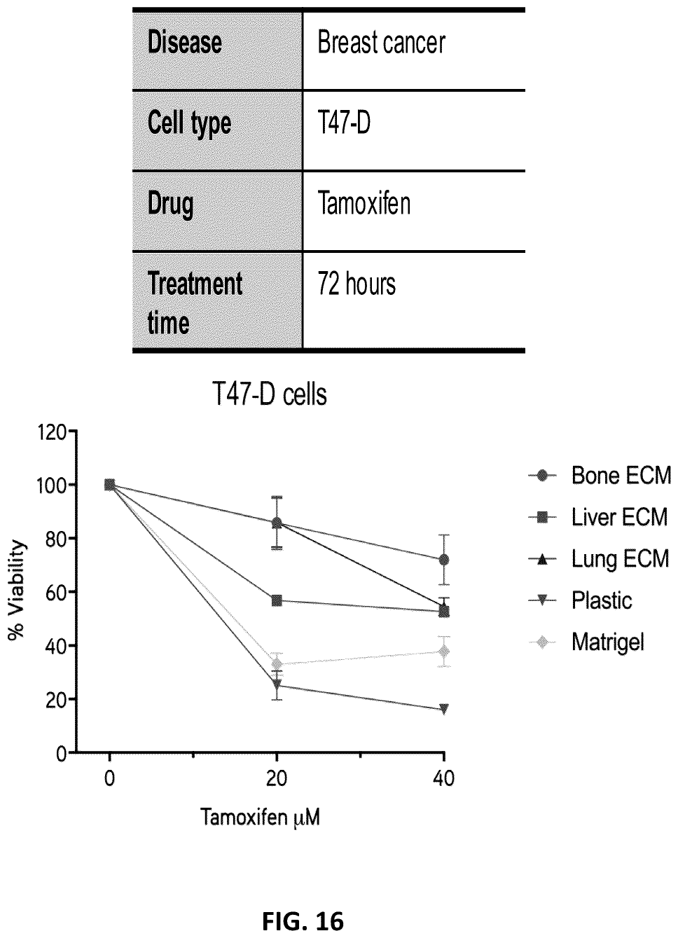

Results. T47-D cells demonstrated differential responses to the same treatment where cells embedded in lung and bone ECM are more resistant than cells embedded in liver, matrigel or plastic being consistent with the literature reporting that high doses of tamoxifen are necessary to treat breast cancer patients with bone metastasis are shown in FIG. 16.

### Example 12—Human Cirrhotic Liver ECM for In Matrico Hepatocellular Carcinoma Model

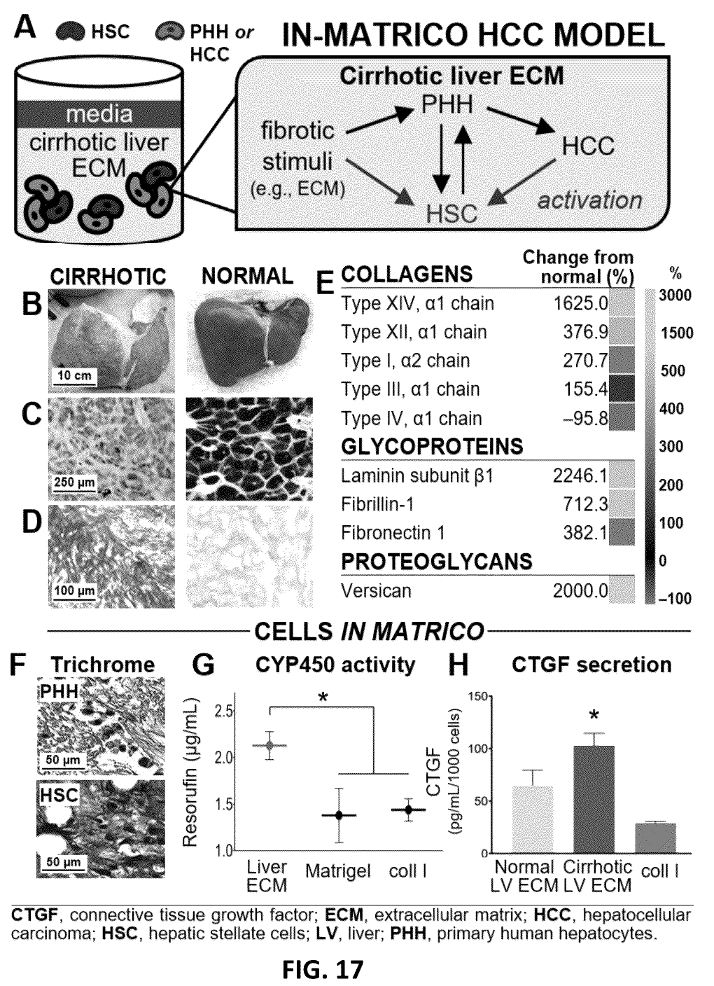

Rationale. Hepatocellular carcinoma (HCC) is a rare cancer with an incidence of 5.84 per 100,000 in USA. Advanced HCC is non-resectable, remains hard to treat, and 5-year survival is only 10%. Patients develop HCC in the cirrhotic liver microenvironment, where cirrhotic extracellular matrix (ECM) contributes to HCC development and regulates interactions between activated hepatic stellate cells and damaged hepatocytes. Current HCC models lack cirrhotic liver ECM and have poor resemblance to the HCC environment. Integrating human cirrhotic liver ECM and liver cells to generate a 3D ‘in-matrico’ HCC model (FIG. 17A) is a significant conceptual and technological advance that can give new insights (biomarkers, targets, drugs) into HCC pathogenesis (still poorly understood) and increase drug discovery success. While current models underperform, advanced predictions for drug responses will be possible using ‘in-matrico’.

Objectives. (1) Identify effects of cirrhotic liver ECM on hepatic stellate cell and hepatocyte phenotypes. (2) Develop and validate in-matrico HCC model that can support clinical trial readiness.

Preliminary Results. Human cirrhotic (alcoholic liver disease) and normal livers (FIG. 17B, n=4) declined for transplant, confirmed Hep A/B/C-COVID-19-, consented for research, procured as if for transplant under approved IRB protocols through UNOS (nationwide organ donation network), and confirmed by liver pathologist using NAS scoring as normal (FO) or cirrhotic (F4), were treated with our patented combination of chemicals, enzymes, and surfactants to remove all cellular components and obtain acellular normal and cirrhotic liver ECMs (FIG. 17C). Trichrome staining showed dramatic deposition of collagens (blue) in cirrhotic liver ECM (FIG. 17D). Matrisome mass spectrometry of cirrhotic liver ECM showed significant changes from normal in collagens, glycoproteins, proteoglycans (FIG. 17E, partial list). Compared to common substrates, monocultures of primary hepatocytes (PHH) and hepatic stellate cells (HSC) in cirrhotic liver ECM (FIG. 17F) showed, respectively, significantly higher hepatocyte CYP450 activity (FIG. 17G) and elevated HSC secretion of connective tissue growth factor (CTGF, FIG. 17H), both consistent with HCC progression (*p<0.05).

Methods. An in-matrico HCC model is used to assess hepatic stellate cell activation and HCC pathogenesis in cirrhotic liver ECM. All liver donors meet standardized acceptance criteria; ECMs subjected to established QC, standardized with negligible lot-to-lot variability. Relative expression quiescence (AMIP-13, Desmin) and activation (Actg2, Loxl1, Col4A1/2, Adamts9, Smad7, Pten) genes in HSC (ScienCell) are evaluated by qRT-PCR, and compared to HSC in normal liver ECM, Matrigel, coll I, plastic. To assess effects of cirrhotic ECM-activated HSC on hepatocyte phenotype, conditioned medium from HSC cultured in cirrhotic liver ECM for 48 h is collected and added to monocultures of primary human hepatocytes (PHH, ScienCell) or hepatocellular carcinoma cell line (Huh 7, ATCC) in cirrhotic and normal liver ECMs. Expression of eighty-eight (88) HCC-associated genes (e.g., Rb1, Myc, Cyclin D1, p53, TGF-β receptor II) is quantified by GeneQuery Human HCC qRT-PCR Array Kit (ScienCell). To evaluate HCC tumor response to standard-of-care drugs (sorafenib, regorafenib) in matrico, spheroid monocultures (HSC) and spheroid co-cultures (HSC-PHH; HSC-Huh7; 1:3 ratio) in cirrhotic and normal liver ECMs are treated for 72 h with clinically therapeutic doses of sorafenib or regorafenib (0.5, 1, 5, 10 μM; control: DMSO vehicle). In clinical trials, HCC patient plasma concentration of sorafenib was 2 mg/L, or 4 μM). At 72 h, drug cytotoxicity is assessed by XTT assay and CTGF secretion by ELISA. Spheroids are fixed, paraffin-embedded, stained for H&E and autophagy flux protein p62 (upregulated in HCC). To validate results, histologic comparisons to HCC specimens from our collaborating tumor bank are performed. All studies have 5 groups: cirrhotic liver ECM, normal liver ECM, Matrigel, collagen I, plastic (control: no ECM), repeated thrice in triplicate. Statistical analyses (Student's t-test, two-way ANOVA) are performed between all groups, p<0.05 significant. Results are validated against patient-derived HCC xenograft model data.

Although the present invention has been described in considerable detail with reference to certain preferred embodiments thereof, other versions are possible. Therefore the spirit and scope of the appended claims should not be limited to the description and the preferred versions contained within this specification. It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, without limiting the true scope and spirit of the invention.

1. A cell culture platform for modeling metastatic cancer comprising:
one or more cell culture vessels comprising a plurality of compartments, each compartment housing a substrate adapted for culturing cells thereon, wherein each substrate comprises a decellularized tissue-specific extracellular matrix derived from tissue of a different anatomical region,
wherein each tissue-specific extracellular matrix comprises a homogenous mixture of macromolecule fragments including collagen, elastin, and glycosaminoglycan.

2. The cell culture platform of claim 1, wherein the tissue-specific extracellular matrix derived from tissue of a different anatomical region of each substrate is selected from the group consisting of bone-specific extracellular matrix, liver-specific extracellular matrix, lung-specific extracellular matrix, brain-specific extracellular matrix, kidney-specific extracellular matrix, skin-specific extracellular matrix, intestine-specific extracellular matrix, heart-specific extracellular matrix, and lymph-specific extracellular matrix.

3. The cell culture platform of claim 1, wherein the plurality of compartments of the one or more cell culture vessels comprise:
a first compartment housing a first substrate;
a compartment housing a second substrate; and
a third compartment housing a third substrate.

4. The cell culture platform of claim 3, wherein:
the first substrate comprises bone-specific extracellular matrix derived from bone tissue;
the second substrate comprises lung-specific extracellular matrix derived from lung tissue; and
the third substrate comprises liver-specific extracellular matrix derived from liver tissue.

5. The cell culture platform of claim 4, wherein the first substrate comprises collagen in a concentration of about 580 to about 620 μg/mL, elastin in a concentration of about 40 μg/mL to 50 μg/mL, and glycosaminoglycan in a concentration of about 10 μg/mL to about 20 μg/mL.

6. The cell culture platform of claim 4, wherein the second substrate comprises collagen in a concentration of about 400 μg/mL to about 530 μg/mL, elastin in a concentration of about 40 μg/mL to 50 μg/mL, and glycosaminoglycan in a concentration of about 3 μg/mL to about 5 μg/mL.

7. The cell culture platform of claim 4, wherein the third substrate comprises collagen in a concentration of about 1100 μg/mL to about 1300 μg/mL, elastin in a concentration of about 120 μg/mL to 150 μg/mL, and glycosaminoglycan in a concentration of about 5 μg/mL to about 15 μg/mL.

8. The cell culture platform of claim 4, wherein:
the first substrate has an elastic modulus of about 6 kPa to about 25 kPa;
the second substrate has an elastic modulus of about 2 kPa to about 12 kPa; and
the third substrate has an elastic modulus of about 2 to about 15 kPa.

9. The cell culture platform of claim 8, wherein:
the first substrate has an elastic modulus of about 6.6 kPa;
the second substrate has an elastic modulus of about 3.1 kPa; and
the third substrate has an elastic modulus of about 2.8 kPa.

10. The cell culture platform of claim 1, wherein, each tissue-specific extracellular matrix is derived from non-metastatic tissue.

11. The cell culture platform of claim 1, wherein each tissue-specific extracellular matrix is derived from fibrotic tissue.

12.-15. (canceled)

16. The cell culture platform of claim 1, wherein:
the one or more cell culture vessels comprise at least one microfluidic chip;
the plurality of compartments comprise a plurality of microfluidic chambers on the at least one microfluidic chip; and
the one or more fluidic channels comprise one or more microfluidic channels fluidly communicating with the plurality of microfluidic chambers.

17. (canceled)

18. The cell culture platform of claim 1, wherein each substrate is individually selected from the group consisting of a surface coating, a hydrogel, a printable bio-ink, one or more fibers, and a media supplement.

19.-47. (canceled)

48. A method of assessing a response of one or more cancer colonies to a drug, the method comprising:
providing one or more cell culture vessels comprising a plurality of substrates arranged in a compartmentalized manner, each substrate comprising a decellularized tissue-specific extracellular matrix derived from tissue of a different anatomical region;
culturing cancer cells to form a cancer colony on each substrate, wherein the cancer cells are foreign to the tissue-specific extracellular matrix of at least one of the plurality of substrates, thereby forming at least one metastatic cancer colony;
contacting each cancer colony with a drug; and
assessing the response by each cancer colony to the drug.

49.-52. (canceled)

53. The method of claim 48, wherein the cancer cells are selected from the group consisting of breast cancer cells and lung cancer cells.

54. The method of claim 48, wherein the tissue-specific extracellular matrix derived from tissue of a different anatomical region of each substrate is selected from the group consisting of bone-specific extracellular matrix, liver-specific extracellular matrix, lung-specific extracellular matrix, brain-specific extracellular matrix, kidney-specific extracellular matrix, skin-specific extracellular matrix, intestine-specific extracellular matrix, heart-specific extracellular matrix, and lymph-specific extracellular matrix.

55. The method of claim 48, where the tissue-specific extracellular matrix of each substrate comprises a homogenous mixture of macromolecule fragments including collagen, elastin, and glycosaminoglycan.

56. (canceled)

57. The method of claim 48, wherein each tissue-specific extracellular matrix is derived from fibrotic tissue.

58.-60. (canceled)

61. The method of claim 48, wherein each substrate is individually selected from the group consisting of a surface coating, a hydrogel, a printable bio-ink, one or more fibers, and a media supplement.

62. A method of assessing cell migration of a primary cancer colony, the method comprising:
providing one or more cell culture vessels comprising:
a first substrate in a first compartment of the one or more cell culture vessels,
one or more second substrates in one or more second compartments of the one or more cell culture vessels,
wherein the first substrate and the one or more second substrates each comprise a decellularized tissue-specific extracellular matrix derived from tissue of a different anatomical region,
wherein the first substrate and the one or more second substrates are arranged in a compartmentalized manner and in fluid communication through one or more fluidic channels;
culturing cancer cells on the first substrate, wherein the cancer cells are native to the tissue-specific extracellular matrix of the first substrate, thereby forming the primary cancer colony,
wherein the one or more fluidic channels are configured to permit cell migration from the first substrate to the one or more second substrates, wherein the cancer cells are adapted to form one or more metastatic cancer colonies on the one or more second substrates; and
assessing the cell migration of the primary cancer colony from the first substrate to the one or more second substrates.

63.-73. (canceled)
